(19) United States
(12) Patent Application Publication
(10) Pub. No.: US 2018/0162913 A1
(43) Pub. Date: Jun. 14, 2018

(54) METHODS FOR POTENTIATING AN IMMUNE RESPONSE USING DEPOT-FORMING AND NON-DEPOT-FORMING VACCINES

(71) Applicant: IMMUNOVACCINE TECHNOLOGIES INC., Halifax, Nova Scotia (CA)

(72) Inventors: Marc MANSOUR, Halifax, Nova Scotia (CA); Genevieve Mary WEIR, Dartmouth, Nova Scotia (CA); Leeladhar SAMMATUR, Halifax, Nova Scotia (CA); Rajkannan RAJAGOPALAN, Dartmouth, Nova Scotia (CA); Marianne STANFORD, Upper Tantallon, Nova Scotia (CA); Lisa Diana MACDONALD, Halifax, Nova Scotia (CA)

(73) Assignee: IMMUNOVACCINE TECHNOLOGIES INC., Halifax, Nova Scotia (CA)

(21) Appl. No.: 15/571,003

(22) PCT Filed: Apr. 27, 2016
(86) PCT No.: PCT/CA2016/050487

§ 371 Date: Oct. 31, 2017

Related U.S. Application Data
(60) Provisional application No. 62/155,677, filed on May 1, 2015.

(51) Int. Cl. C07K 14/025; A61K 39/00; A61K 47/24; A61K 47/26; A61K 47/28; A61K 9/00; A61K 9/127; A61K 39/12; A61P 35/00

(52) U.S. Cl. CPC C07K 14/025; A61K 39/0011; A61K 47/24; A61K 47/26; A61K 47/28; A61K 9/0019; C12N 2710/20034; A61K 39/12; A61P 35/00; A61K 2039/55561; A61K 2039/55555; A61K 2039/572; C12N 2710/20023; A61K 9/127

(57) ABSTRACT

The present disclosure provides methods, vaccines and kits for potentiating an immune response to an antigen in a subject. The methods comprise administering to the subject at least one dose of a depot-forming vaccine comprising one or more antigens in a hydrophobic carrier; and subsequently administering to the subject at least one dose of a non-depot-forming vaccine comprising the one or more antigens.

### CROSS-REFERENCE TO RELATED APPLICATIONS

This application claims the benefit of and priority from U.S. Provisional Patent Application No. 62/155,677 filed on May 1, 2015, which is hereby incorporated by reference in its entirety.

### FIELD

The present invention relates generally to methods for potentiating an immune response and, in particular, to methods involving a prime-boost strategy using a depot-forming vaccine and a non-depot-forming vaccine.

### BACKGROUND

In immunotherapy, and cancer immunotherapy in particular, generating sufficiently potent immune responses to vaccine antigens is a major obstacle. The immune responses to cancer vaccine antigens are often hampered by immune tolerance mechanisms, which originate within the tumor as a mechanism of immune escape (Kim et al, Immunology, 2007).

Highly purified and synthetic antigens, such as peptides, are often poorly immunogenic and thus require immune stimulants such as adjuvants to facilitate robust immune responses (Irvine, Nat Mater, 2013). This requirement was demonstrated in a clinical trial conducted in ovarian cancer patients, who were immunized with NY-ESO-1(81-100) peptide prepared either in an aqueous buffer, or in an oil-in-water emulsion with Montanide ISA51 VG oil, or in an oil-in-water emulsion with Montanide ISA51 VG oil and TLR agonist adjuvant (Tsuji et al, Cancer Immunol Res, 2013; Sabbatini et al, Clin Can Res, 2012). In this trial, patients that were vaccinated with this peptide in an aqueous buffer did not develop antigen-specific immune responses as detected by antigen-specific antibody production or antigen-specific CD8+ or CD4+ T cells (assayed by ELISA, ELISPOT, intracellular staining and tetramer staining). However, patients immunized with Montanide ISA51 VG oil emulsions, with or without the TLR agonist adjuvant, developed strong B cell and T cell immune responses towards the antigen. The Montanide ISA51 water-in-oil emulsion provided a depot effect for the antigens, increasing their stability in vivo and slowly releasing antigen to the immune system, as the emulsion breaks down in vivo. The maintenance and detection of immune responses required the repeated immunization with a Montanide ISA51 water-in-oil emulsion vaccine.

Other Montanide oil-based formulations (Karkada et al, J Immunother, 2010) are able to induce rapid, strong and sustained antigen-specific immune responses towards cancer vaccine antigens with a single immunization.

Maintaining immune responses, for example in the context of tumor-induced immune suppression, often requires multiple boosting immunizations. Although oil-based formulations can be used for booster immunizations, re-stimulation of the immune system with such vaccine formulation can lead to increased and problematic reactions at the vaccine site, due to the combination of a strong systemic immune response and a sustained antigen depot. This is a common observation in clinical trials using such vaccines and has been documented (Stills, ILAR, 2005, Sabbatini et al, Clin Can Res, 2012).

In the present disclosure, we report novel methods for potentiating an immune response to one or more antigens, as well as vaccine compositions that can be used in the described methods.

### SUMMARY

In an embodiment, the present disclosure relates to a method for potentiating an immune response to an antigen in a subject, said method comprising:

(i) administering to the subject at least one dose of a depot-forming vaccine comprising one or more antigens in a hydrophobic carrier; and

(ii) subsequently administering to the subject at least one dose of a non-depot-forming vaccine comprising the one or more antigens.

In another embodiment, the present disclosure relates to the use of a depot-forming vaccine in combination with a non-depot-forming vaccine for potentiating an immune response against an antigen, wherein at least one dose of the depot-forming vaccine comprising the antigen and a hydrophobic carrier is for administration prior to the non-depot-forming vaccine comprising the antigen.

In another embodiment, the present disclosure relates to a kit comprising: at least one container comprising a depot-forming vaccine, said depot-forming vaccine comprising one or more antigens and a hydrophobic carrier; and at least one container comprising a non-depot-forming vaccine, said non-depot-forming vaccine comprising the one or more antigens.

In another embodiment, the present disclosure relates to a kit comprising: at least two containers, each container comprising one or more antigens, a T-helper epitope, an adjuvant and lipids; at least one container comprising a hydrophobic carrier; and at least one container comprising an aqueous carrier, wherein at least one container of antigen, T-helper epitope, adjuvant and lipids is for reconstitution with the hydrophobic carrier to prepare a depot-forming vaccine and at least one container of antigen, T-helper epitope, adjuvant and lipids is for reconstitution with the aqueous carrier to prepare a non-depot-forming vaccine.

In another embodiment, the present disclosure relates to a combination of a depot-forming vaccine comprising one or more antigens and a hydrophobic carrier, and a non-depot-forming vaccine comprising the one or more antigens, for use in a method as described herein.

Other aspects, embodiments and features of the present invention will become apparent to those of ordinary skill in the art upon review of the following description in conjunction with the accompanying claims and figures.

### BRIEF DESCRIPTION OF THE FIGURES

In the figures, which illustrate embodiments of the invention by way of example only:

FIG. 1 illustrates the IFN-gamma ELISPOT responses of HHD-DR1 mice vaccinated with an antigen contained in either oil based (group 1) or aqueous based (group 2) formulations. Immune responses were measured eight days after vaccination by stimulating lymph node cells with syngeneic dendritic cells without peptide (background) or loaded with an irrelevant HLA-A2 restricted peptide (ALMEQQHYV; SEQ ID NO: 1) or the survivin HLA-A2 restricted peptide (SurA2.M, LMLGEFLKL; SEQ ID NO: 2) in an IFN-gamma ELISPOT plate. Statistical analysis was performed by 2-way ANOVA with Bonferroni post test comparing the response to the SurA2.M peptide with the response to the irrelevant peptide; ***p<0.001, NS (not significant) p>0.05.

FIG. 2 illustrates the IFN-gamma ELISPOT responses of HHD-DR1 mice primed with survivin peptides formulated in an oil based vaccine and then boosted with survivin peptides in either an oil based vaccine (group 1) or an aqueous based vaccine (group 2). Immune responses were measured eight days after boost immunization by stimulating splenocytes with media (background), or HLA-A2 restricted survivin peptide (SurA2.M, LMLGEFLKL; SEQ ID NO: 2), or an irrelevant HLA-A2 restricted peptide (ALMEQQHYV; SEQ ID NO: 1), on an IFN-gamma ELISPOT plate. Statistical analysis was performed by 2-way ANOVA with Bonferonni post test comparing response to SurA2.M peptide with response to irrelevant peptide; *p<0.05, ****p<0.0001.

FIG. 3 illustrates the IFN-gamma responses of mice primed with an oil based vaccine and boosted with oil based vaccine or aqueous vaccine with concurrent metronomic cyclophosphamide treatment. All mice were treated with metronomic cyclophosphamide (20 milligrams/kilogram/day) for one week on, one week off, and vaccinated on days 0, 21 and 42. Mice in group 1 were vaccinated three times with oil based vaccine. Mice in group 2 were vaccinated twice with oil based vaccine and once with aqueous vaccine. Mice in group 3 were vaccinated once with oil based vaccine and twice with aqueous vaccine. Immune responses were measured eight days after last immunization (day 50) by stimulating splenocytes with media (background), or HPV16E7₄₉₋₅₇ peptide (R9F, RAHYNIVTF; SEQ ID NO: 3), or an irrelevant peptide (RMFPNAPYL; SEQ ID NO: 4), on an IFN-gamma ELISPOT plate. Results shown as average response ±SEM. Statistical analysis was performed by 1-way ANOVA with Tukey post test comparing group responses to R9F peptide.

FIG. 4 illustrates the efficacy of treatment with an oil based vaccine, an aqueous vaccine, and metronomic cyclophosphamide for the treatment of cancer in a mouse model. Mice were implanted with C3 tumors on study day 0. Mice in group 1 remained untreated. Mice in groups 2-4 were treated with metronomic cyclophosphamide (20 mg/kg/d) for one week on and one week off alternating starting on study day 5. These groups were also vaccinated on study days 12, 33 and 54. Mice in group 2 were vaccinated three times with oil based vaccine, mice in group 3 were vaccinated twice with oil based vaccine and once with aqueous vaccine, mice in group 4 were vaccinated once with oil based vaccine and twice with aqueous vaccine. FIG. 4A shows average tumor volume per group ±SEM. FIG. 4B shows percent survival.

FIG. 5 shows that a boost immunization is required to maintain or increase immune responses induced by an oil based vaccine. Mice were vaccinated once (group 1) or twice (group 2) with an oil based vaccine containing SurA2.M peptide (LMLGEFLKL; SEQ ID NO: 2). Immune responses detected by IFN-gamma ELISPOT eight days after final immunization. Mice in group 3 were vaccinated twice with an oil based vaccine containing the SurA2.M peptide and immune responses detected by IFN-gamma ELISPOT 27 days after final immunization. Irrelevant HLA-A2 restricted peptide (ALMEQQHYV; SEQ ID NO: 1) or media only (background) served as negative controls. Statistical analysis was performed by 1-way ANOVA with Tukey post-test comparing response to SurA2.M stimulation of each group; *p<0.05, **p<0.01.

FIG. 6 depicts a Phase 1b clinical trial design to evaluate the safety and immunogenicity of DPX-Survivac (Oil) to prime an immune response, followed by DPX-Survivac (Aqueous) to boost and maintain immune responses. DPX-Survivac (Oil) was administered (0.25 milliliter dose) on study days 0 and 28. DPX-Survivac (Aqueous) was administered (0.50 milliliter dose) on study days 56, 84, and 112. Low dose cyclophosphamide (50 milligram dose twice a day, Baxter) was given by oral administration for 7 consecutive days every 14 days, commencing 7 days prior to the first vaccination (study day −7) and completing after 19 weeks. Blood samples were collected prior to the first vaccination (baseline) and then on study days 0, 28, 42, 56, 84 and 112 to isolate and cryo-preserve peripheral blood mononuclear cells (PBMCs). Blood samples were also collected at later time points when possible. PBMCs were used for immunological assays including ELISPOT and multimer flow cytometry.

FIG. 7 shows the immune responses detected between study days 0-112 in subject 03-28 vaccinated with DPX-Survivac (Oil) and boosted with DPX-Survivac (Aqueous). Subject was treated with low dose cyclophosphamide, DPX-Survivac (Oil) (black arrows) and DPX-Survivac (Aqueous) (outlined arrows) as outlined in FIG. 6. Immune responses were measured by IFN-gamma ELISPOT (A) and multimer stain for SurA2.M-specific T cells (B) using PBMC isolated from blood collected on study days 0, 28, 42, 56, 84, 112.

FIG. 8 shows the immune responses detected between study days 0-112 in subject 03-30 vaccinated with DPX-Survivac (Oil) and boosted with DPX-Survivac (Aqueous). Subject was treated with low dose cyclophosphamide, DPX-Survivac (Oil) (black arrows) and DPX-Survivac (Aqueous) (outlined arrows) as outlined in FIG. 6. Immune responses were measured by IFN-gamma ELISPOT (A) and multimer stain for SurA1.T-specific T cells (B) using PBMC isolated from blood collected on study days 0, 28, 42, 56, 84, 112.

### DETAILED DESCRIPTION

The development of a strong and prolonged immune response often requires the repeated administration of a vaccine, particularly in cancer immunotherapy and/or for the development of a strong cellular immune response. A major challenge for immunologists has been the development of vaccines and methods of administration which potentiate the immune response and avoid immune tolerance mechanisms, without significant adverse effects. Despite some success, it has become apparent that certain antigens, such as “weakly immunogenic antigens” as described herein, pose problems for current vaccination approaches. Also, certain types of diseases, such as for example cancer, are often able to resist the cellular and humoral immunity that is characteristically generated by conventional vaccines and traditional administration strategies.

Maintaining immune responses, for example in the context of tumor-induced immune suppression, often requires multiple immunizations. However, re-stimulation of the immune system with oil-based formulations can lead to increased and problematic reactions at the vaccine injection site, due to the combination of a strong systemic immune response and a sustained antigen depot. This is a common observation in clinical trials using such vaccines and has been documented (Stills, ILAR, 2005, Sabbatini et al, Clin Can Res, 2012).

We have also observed adverse events at the site of immunization after repeated injections of a Montanide ISA51 based vaccine containing survivin antigen in an ovarian cancer Phase 1 study. We found that injection site reactions are mediated by the persistent infiltration of T cells and other immune cells at the site of the vaccine deposit. These injection site immune reactions pose difficulties in the development of clinically useful vaccines and vaccine administration strategies.

The present disclosure provides methods and vaccines for potentiating an immune response to an antigen in a subject. The disclosed methods involve a modified strategy of vaccination which includes priming with a depot-forming vaccine to induce robust antigen-specific immune responses and, to further reduce injection site reactions (primarily mediated by the hydrophobic, e.g. oil, component of the depot-forming vaccine), maintaining and/or boosting the immune responses with a non-depot-forming vaccine formulation containing the same antigens. Whereas the non-depot-forming (e.g. aqueous) vaccine was incapable of priming an immune response, we surprisingly found that it was capable of maintaining, and even boosting, an immune response in subjects that have been previously primed with a depot-forming (e.g. oil-based) vaccine. These results have been demonstrated both in pre-clinical studies in mice and in human clinical trials.

In an embodiment, the method disclosed herein comprises: (i) administering to the subject at least one dose of a depot-forming vaccine comprising one or more antigens in a hydrophobic carrier; and (ii) subsequently administering to the subject at least one dose of a non-depot-forming vaccine comprising the one or more antigens. In an embodiment, the subject has had no prior immune response towards the antigen in the vaccine.

As used herein, “potentiating” or “to potentiate” means that the immune response to the antigen is made more effective or an adverse event is avoided, abolished or lessened in strength and/or duration. By “more effective”, it is meant that the immune response is enhanced, elevated, improved, strengthened or prolonged to the benefit of the subject relative to the prior immune response status of the subject.

In some embodiments, “potentiating” means that there is an improved efficacy in inducing, eliciting or generating an immune response to the antigen. As used herein, “improved efficacy”, “improving the efficacy” or the like refers to any change or alteration in the immune response of a subject that is capable of rendering the vaccine more effective in treating a disease or disorder. In some embodiments, this may involve accelerating the appearance of an immune response and/or improving the persistence or strength of an immune response.

In some embodiments, “potentiating” refers to the ability to induce, strengthen or prolong an antigen-specific recall response in a subject that has previously been primed by immunization with a depot-forming vaccine. As opposed to a primed immune response that occurs upon first exposure to an antigen, a recall immune response is the immune response occurring on the second and/or subsequent exposures to an antigen, re-establishing an immune response that was previously produced by a prime immunization.

In some embodiments, “potentiating” refers to the ability to maintain and/or boost an antigen-specific immune response in a subject that has previously been primed by immunization with a depot-forming vaccine. By “maintain and/or boost”, it is meant that the previously induced immune response is enhanced, elevated, improved, strengthened or prolonged to the benefit of the subject.

In some embodiments, “potentiating” refers to the ability to reduce the occurrence of an adverse event. For example, in an embodiment, potentiating the immune response may involve a reduction in the occurrence of injection site reactions caused by single or repeated administration of a depot-forming vaccine. In this embodiment, the immune response may be potentiated by priming the immune response with a depot-forming vaccine and maintaining and/or boosting the immune response with a non-depot-forming vaccine.

The depot-forming and non-depot-forming vaccines which can be used in the methods disclosed herein are described below. In particularly suitable embodiments, the depot-forming vaccine is a water-free or substantially water-free oil-based vaccine and the non-depot-forming vaccine is an aqueous vaccine.

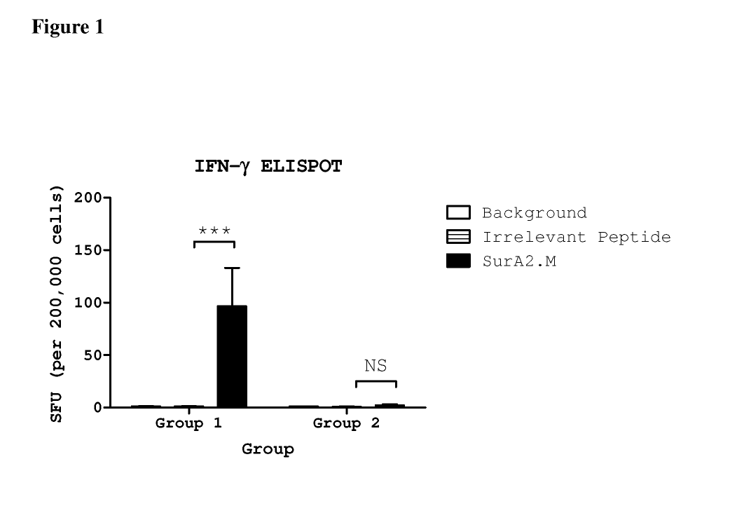

It has been surprisingly and unexpectedly been found that the administration of at least one or two priming doses of a depot-forming vaccine is sufficient to generate an immune response that can be maintained and/or boosted by a non-depot-forming vaccine. This is surprising because it is known that aqueous-based vaccines (i.e. non-depot-forming vaccines) often do not work to generate an immune response, particularly in the field of cancer or for the administration of weakly immunogenic antigens. This is highlighted by Example 1 and FIG. 1 herein in which it was observed that an aqueous-based vaccine was not capable of generating an antigen-specific immune response (Group 2), whereas an oil-based vaccine (i.e. depot-forming vaccine) having the same antigen was able to induce an antigen-specific immune response (Group 1).

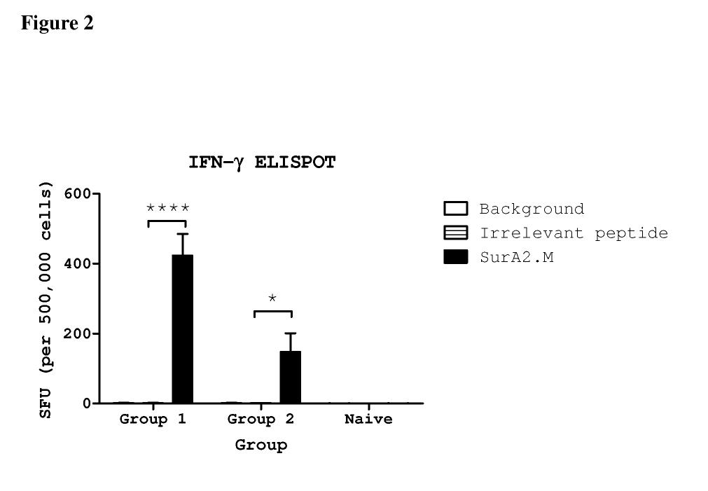

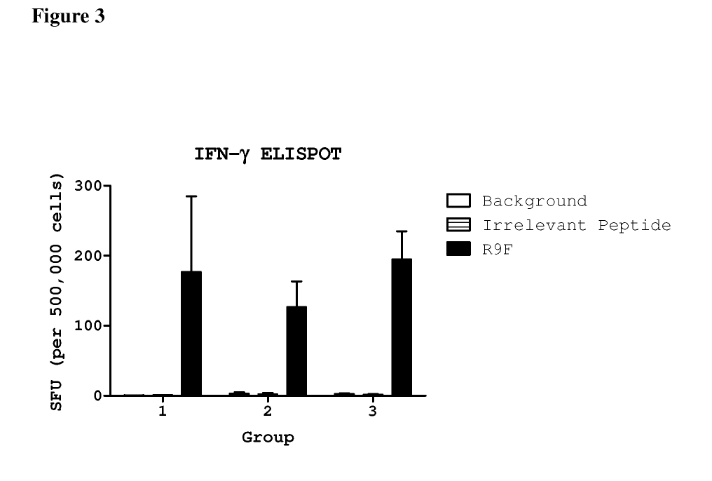

However, in Examples 2 and 3 where one or two priming doses of an oil-based depot-forming vaccine are administered prior to boosting with either the same oil-based vaccine or an aqueous-based vaccine, it was found that the aqueous-based vaccine was an adequate formulation for maintaining and/or boosting the immune response (see FIGS. 2 and 3). Indeed, in FIG. 3 it is shown that after just one priming dose with the oil-based vaccine, two boosting doses of the aqueous-based vaccine generated an equivalent immune response as the treatment group where all vaccinations were done using the oil-based vaccine (compare Group 3 and Group 1, respectively).

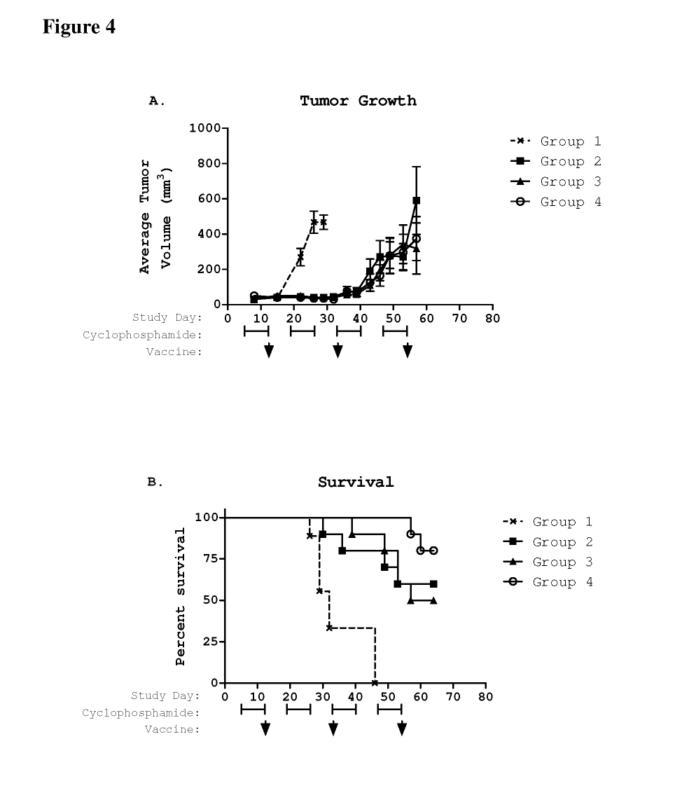

These surprising and unexpected results were found to translate to an effective therapeutic benefit in the protection from tumor growth. Example 4 demonstrates that priming with an oil-based vaccine and then boosting with an aqueous-based vaccine containing the same antigen provide an equivalent or better protection from tumor growth than treatment with all vaccinations being the oil-based vaccine (compare Group 4 and Group 2, respectively; FIGS. 4A and 4B).

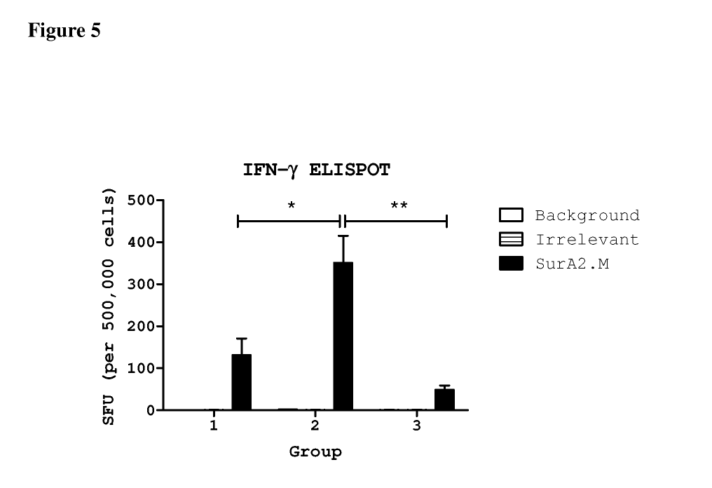

The importance of administering repeated booster immunizations is further highlighted by Example 5 which demonstrates that an antigen-specific immune response induced by a vaccine declines over time without boosting. As shown in FIG. 5, the immune response dropped significantly within 27 days following the administration of a booster vaccination. This establishes the importance of further booster immunizations to maintain and/or boost the immune response.

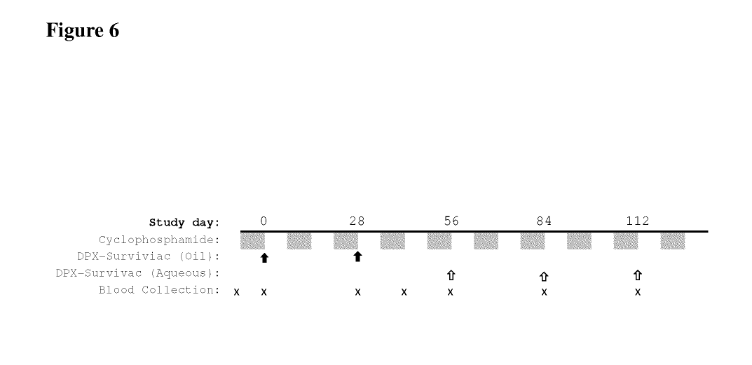

We have also demonstrated, by human clinical trials, that the methods disclosed herein are surprisingly effective in inducing and maintaining/boosting an antigen-specific immune response in HLA-A1 and HLA-A2 positive subjects. In Examples 6-8, an exemplary depot-forming vaccine (DPX-Survivac (Oil)) was capable of priming an immune response in both a HLA-A1+ and HLA-A2+ human subject with no prior immune response to the vaccine antigen. Subsequent boost immunizations with an exemplary non-depot-forming vaccine (DPX-Survivac (Aqueous)) were capable of maintaining, even at an elevated level, these immune responses (FIGS. 6-8).

By priming with a depot-forming vaccine and subsequently maintaining and/or boosting the immune response with a non-depot-forming vaccine, this should also reduce the occurrence of injection site reactions against the depot-forming vaccine. The non-depot-forming vaccine (e.g. aqueous-based vaccine) will be cleared from the injection site faster than the depot-forming vaccine (e.g. oil-based vaccine) thereby potentially alleviating injection site reactions. Moreover, in embodiments of the present disclosure in which lipid amphiphiles are used in both the depot-forming and non-depot-forming vaccines, the lipids present in the non-depot-forming vaccine may act as an attractant for antigen presenting cells.

For the patients outlined in the examples who received the non-depot-forming vaccine after priming doses of the depot-forming vaccine, it was indeed observed that all injection site reactions were mild (grade 1) and consisted of erythema, pruritus, induration and pain. Also, the majority of these site reactions were completely resolved two to three months later, particularly if the injection site received the aqueous formulation.

Methods for Potentiating the Immune Response

Methods of the invention for potentiating an immune response in a subject comprise the combined administration of at least one dose of a depot-forming vaccine comprising one or more antigens in a hydrophobic carrier and, subsequently, at least one dose of a non-depot-forming vaccine comprising the one or more antigens.

The depot-forming vaccine is described herein below. A particularly suitable embodiment of the depot-forming vaccine is a water-free or substantially water-free oil-based vaccine.

Generally, each of the at least one dose of the depot-forming vaccine is a priming dose that is capable of inducing an immune response to the one or more antigens. As used herein, a “priming dose” refers to the ability of the vaccine to initiate an immune response to the one or more antigens. This is in contrast to a recall immune response which maintains and/or boosts a pre-existing immune response to an antigen. It will be appreciated that, as used herein, the term “priming dose” encompasses not only the first administration (e.g. first exposure) of the antigen using the depot-forming vaccine, but may also encompass one or more subsequent administrations that are also used to initiate the immune response to the antigen, so long as these administrations are before the first administration of the non-depot-forming vaccine.

It is within the ability of the skilled person to determine the number, duration and interval between administrations of the priming doses of the depot-forming vaccine so as to induce an immune response to the antigen. These features may, for example, be dependent upon the antigen used, the immune response (humoral or cellular) and/or the disease or disorder to be treated.

In some embodiments, the at least one dose of the depot-forming vaccine is one, two, three, four or five doses. In a more particular embodiment, the at least one dose of the depot-forming vaccine is one or two doses, and in a further embodiment it is only one dose. The number of doses should be sufficient to induce an immune response to the antigen.

The interval between administrations of the depot-forming vaccine should be close together enough that a significant decline in the induction of the immune response does not occur. In some embodiments, after a first dose of the depot-forming vaccine, each subsequent dose of the depot-forming vaccine is administered within about 1 day, 1 week, 2 weeks, 3 weeks, 4 weeks, 5 weeks, 6 weeks, 7 weeks, 8 weeks, 9 weeks or 10 weeks of the immediately preceding dose. In a more particular embodiment, each subsequent dose of the depot-forming vaccine is administered within about 1 day, 1 week, 2 weeks, 3 weeks, or 4 weeks of the immediately preceding dose, and in a further embodiment it is administered about 3 or 4 weeks after the immediately preceding dose.

The duration of time over which the depot-forming vaccine may be administered, from first dose to last dose, should be sufficient to initiate an immune response to the antigen but not so long as to encounter adverse effects, such as injection site reactions to the depot-forming vaccine. In some embodiments, the duration of time from first to last dose of the priming doses of the depot-forming vaccine, is 0 days (i.e. single administration), 1 week, 2 weeks, 3 weeks, 4 weeks (1 month), 2 months or 3 months. In a more particular embodiment, the duration is 3 or 4 weeks.

In conjunction with the priming doses of the depot-forming vaccine, the depot-forming vaccine may, in some embodiments, additionally be administered to the subject at another time when it is not used as a priming dose. For example, the depot-forming vaccine may be administered during or after the course of treatment with the non-depot-forming vaccine. In these embodiments, the depot-forming vaccine would be used as a maintenance or boosting dose. It is within the ability of the skilled person to determine the number, duration and interval between administrations of these additional doses of the depot-forming vaccine. In some embodiments, the number, duration and interval may be the same as those described above for the priming doses of the depot-forming vaccine, with the exception that the number of doses would not be based on the ability to initiate or induce an immune response to the antigen.

In the methods disclosed herein, the non-depot-forming vaccine is administered subsequent to at least one dose of the depot-forming vaccine. By “subsequently administering” or “administered subsequent to”, it is meant that the non-depot-forming vaccine is administered at a time after at least one dose of the depot-forming vaccine. In some embodiments, “subsequently administering” means that the non-depot-forming vaccine is not administered until at least some measurable or detectable level of an immune response to the antigen could be detected.

Generally, each of the at least one dose of the non-depot-forming vaccine is a maintenance or boosting dose that is capable of maintaining and/or boosting the immune response to the one or more antigens. As used herein, a “maintenance or boosting dose” refers to the ability of the vaccine to sustain, prolong and/or enhance an immune response to the one or more antigens. It is not necessary that the immune response be sustained at the same level as previously observed or measured. As used herein, to “maintain and/or boost” the immune response encompasses embodiments where the immune response is maintained at a level sufficient to provide a therapeutic benefit to the subject. As will be appreciated, there may be periods between vaccine administrations where the strength of the immune response decreases, perhaps even to an undetectable or unmeasurable level. In such instances, to “maintain and/or boost” the immune response refers the ability of the non-depot-forming vaccine to effectively induce a recall response from a pre-existing immune response to the antigen.

It is within the ability of the skilled person to determine the number, duration and interval between administrations of the maintenance or boosting doses of the non-depot-forming vaccine so as to maintain and/or boost the immune response to the antigen. These features may, for example, be dependent upon the antigen used, the immune response (humoral or cellular) and/or the disease or disorder to be treated.

In some embodiments, the at least one dose of the non-depot-forming vaccine is one, two, three, four or five doses. In another embodiment, the at least one dose of the non-depot-forming vaccine is a continuous repeated dosing. By “continuous repeated dosing” it is meant that administration of the non-depot-forming vaccine continues for any number of administrations until a decision is made to discontinue treatment. During the duration of the continuous repeated dosing, there may be changes in the frequency (interval) of administration of the non-depot-forming vaccine. In an embodiment, the at least one dose of the non-depot-forming vaccine is a continuous repeated dosing once every day, once every week, once every two weeks, once every three weeks, or once monthly.

The administrations of the non-depot-forming vaccine should be close together enough that it remains possible to induce a recall response to the antigen. In some embodiments, after a first dose of the non-depot-forming vaccine, each subsequent dose of the non-depot-forming vaccine is administered within about 1 day, 1 week, 2 weeks, 3 weeks, 4 weeks, 5 weeks, 6 weeks, 7 weeks, 8 weeks, 9 weeks or 10 weeks of the immediately preceding dose. In a more particular embodiment, each subsequent dose of the depot-forming vaccine is administered within about 1 day, 1 week, 2 weeks, 3 weeks, or 4 weeks of the immediately preceding dose, and in a further embodiment it is administered about 3 or 4 weeks after the immediately preceding dose.

The first dose of the non-depot-forming vaccine is administered subsequent to at least one dose of the depot-forming vaccine. There may be an overlap in the administration of the priming doses of the depot-forming vaccine and the maintenance or boosting doses of the non-depot-forming vaccine, so long as at least one administration of depot-forming vaccine occurs before the first dose of the non-depot-forming vaccine.

The final priming dose of the depot-forming vaccine and the first dose of the non-depot-forming vaccine should be close together enough that it remains possible to induce a recall response to the antigen. In some embodiments, the methods herein comprise administering a first maintenance or boosting dose of the non-depot-forming vaccine within about 1 day, 1 week, 2 weeks, 3 weeks, 4 weeks, 5 weeks, 6 weeks, 7 weeks, 8 weeks, 9 weeks or 10 weeks of a final priming dose of the depot-forming vaccine. In a more particular embodiment, a first maintenance or boosting dose of the non-depot-forming vaccine is administered within about 1 day, 1 week, 2 weeks, 3 weeks, or 4 weeks of a final priming dose of the depot-forming vaccine, and is a further embodiment it is within about 3 weeks.

The duration of time over which the non-depot-forming vaccine may be administered, from first dose to last dose, may encompasses any period of time in which the subject is in need thereof, such as for the treatment of a particular disease or disorder. In some embodiments, the duration of time from first to last dose of the of the depot-forming vaccine, is 0 days (i.e. single administration), 1 week, 2 weeks, 3 weeks, 1 month, 2 months, 3 months, 6 months, 9 months, 12 months, 2 years, 3 years, 4 years, 5 years, or more.

In an embodiment, the methods disclosed herein comprise administering two doses of the depot-forming vaccine prior to administration of the non-depot-forming vaccine. As shown in Example 2, two priming doses (days 0 and 21) of the depot-forming vaccine are sufficient to induce an antigen-specific immune response that can later be boosted by either depot-forming vaccine or non-depot-forming vaccine (day 84). As shown in Examples 6-8 (FIG. 6), two priming doses (days 0 and 28) of the depot-forming vaccine are sufficient to induce an antigen-specific immune response that can later be maintained/boosted by non-depot-forming vaccine (days 56, 84 and 112).

In an embodiment, the methods disclosed herein comprise administering only one dose of the depot-forming vaccine prior to administration of the non-depot-forming vaccine. As shown in Example 1 (Group 1) and Example 3 (Group 3), one priming dose of the depot-forming vaccine is sufficient to induce an antigen-specific immune response that can later be boosted by non-depot-forming vaccine (Example 3).

In an embodiment of the methods disclosed herein, a priming dose of the depot-forming vaccine is administered on day 0 and day 21. In another embodiment, a priming dose of the depot-forming vaccine is administered on day 0 and day 28.

In an embodiment of the methods disclosed herein, a priming dose of the depot-forming vaccine is administered on day 0 and day 21, and the non-depot-forming vaccine is administered at least on day 42. In this embodiment, administration of the non-depot-forming vaccine may continue once every three weeks after day 42.

In an embodiment of the methods disclosed herein, a priming dose of the depot-forming vaccine is administered on day 0 and day 28, and the non-depot-forming vaccine is administered at least on day 56. In this embodiment, administration of the non-depot-forming vaccine may continue once every four weeks after day 56.

In an embodiment of the methods disclosed herein, a priming dose of the depot-forming vaccine is administered only on day 0.

In an embodiment of the methods disclosed herein, a priming dose of the depot-forming vaccine is administered only on day 0, and the non-depot-forming vaccine is administered at least on day 42. In this embodiment, administration of the non-depot-forming vaccine may continue once every three weeks after day 42.

Agent that Interferes with DNA Replication

The methods disclosed herein may also comprise administering an agent that interferes with DNA replication. In a particular embodiment, an agent that interferes with DNA replication is administered when the methods disclosed herein are used in the treatment or prevention of cancer.

Exemplary embodiments of such agents and methods of use thereof are described, for example, in WO2014/153636.

As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The methods according to the invention encompass the use of any agent that interferes with DNA replication by any means known in the art. In an exemplary embodiment, and without limitation, the agent that interferes with DNA replication is a drug.

In an embodiment, the agent that interferes with DNA replication is one which, when used at doses that are non-chemotherapeutic, is capable of selectively affecting DNA replication in cells of the immune system, with the intent of modulating the immune system to enhance vaccine responses. By “non-chemotherapeutic”, it is meant that the dose of the agent is a dose lower than that which would be used to directly and selectively destroy malignant or cancerous cells and tissues.

Other embodiments of an agent that interferes with DNA replication include agents that interfere with DNA replication to cause programmed cell death, with the ability to selectively target rapidly dividing cells of the immune system. The purpose of such agents is to modulate cells of the immune system to enhance vaccine responses. Such agents are typically used at doses that are not expected to be chemotherapeutic and are considered acceptable for use in humans. The purpose of selectively targeting immune cells may be to reduce the number of immune suppressive cells, and/or deplete useful immune cells involved in mediating the immune response for the purposes of inducing rapid proliferation upon removal of the drug targeting DNA replication.

Interference with DNA replication leading to cell death may be caused by numerous mechanisms, including but not limited to, the formation of DNA cross-linking (e.g. by alkylating agents, platinum compounds, etc.), methylation of DNA (i.e. by methylating agents), base substitution (i.e. by nucleoside analogs). Exemplary agents and their mechanisms are described in Cancer Chemotherapy and Biotherapy: Principles and Practice (Cabner B. A., 5^{th} edition, Lippincott Williams & Wilkins, PA, USA, 2011).

In an embodiment, the agent that interferes with DNA replication is an alkylating agent. Alkylating agents include, but are not limited to, cyclophosphamide, temozolomide, ifosfamide, mafosfamide, melphalan, busulfan, bendamustine, uramustine, carmustine or bis-chloroethylnitrosourea (BCNU), chlorambucil, mitomycin C, and their derivatives, active metabolites or metabolite intermediates. A suitable derivative may be, for example and without limitation, palifosfamide (e.g. a derivative of ifosfamide).

In another embodiment, the agent that interferes with DNA replication is a platinum compound. Platinum compounds include, but are not limited to, carboplatin, cisplatin, oxaliplatin and their derivatives.

In another embodiment, the agent that interferes with DNA replication is a methylating agent. Methylating agents include, but are not limited to, temzolomide, procarbazine and dacarbazine, and their derivatives.

In another embodiment, the agent that interferes with DNA replication is a nucleoside analog. Non-limiting examples of nucleoside analogs include gemcitabine, 5-fluorouracil, cytosine arabinoside (Ara-C) and their derivatives.

In another embodiment, any drug that inhibits DNA replication indirectly by inhibiting enzymes critical to DNA replication, such as topoisomerase I, topoisomerase II or DNA polymerase, may also be used. Such drugs include, for example and without limitation, doxorubicin, daunorubicin, mitoxantrone, etoposide, teniposide, topotecan, camptothecin, irinotecan, acyclovir and ganciclovir.

Exemplary agents that interfere with DNA replication, and which may be used in the methods of the invention include, without limitation, those listed below in Table 1. As the skilled person will appreciate, these are examples of agents that may be used. Additional agents include, for example, any drug or compound that interferes with DNA replication by a similar mechanism and/or that has a similar functional group.

TABLE 1

| DNA Replication | | | |
|---|---|---|---|
| Inhibitor | Functional group | Description | Exemplary Agents |
| Alkylating agents | Nitrogen mustard | Alkylate DNA | Cyclophosphamide |
| | (bischloroethylamine) | | Ifosfamide |
| | RN(CH₂CH₂Cl)₂ | | Mafosfamide |
| | | | Melphalan |
| | | | Bendamustine |
| | | | Uramustine |
| | | | Palifosfamide |
| | | | Chlorambucil |
| | | | 4-Hydroxycyclophosphamide |
| Alkylating agents | Nitrosourea | Alkylate DNA | 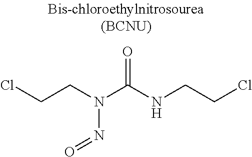 [SMILES: O=NN(CCCl)C(=O)NCCCl] |
| Alkylating agents | Alkyl sulfonates | Alkylate DNA | 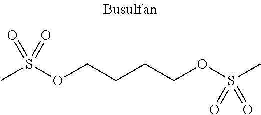 |
| Antitumor Antibiotics | Aziridines or Ethylene imines | Alkylate DNA and Intercalate DNA | 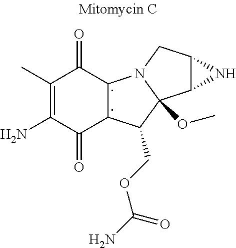 [SMILES: CO[C@@]12[C@H](COC(N)=O)C3C(=O)C(N)=C(C)C(=O)C3N1C[C@@H]1N[C@@H]12] |
| | | | 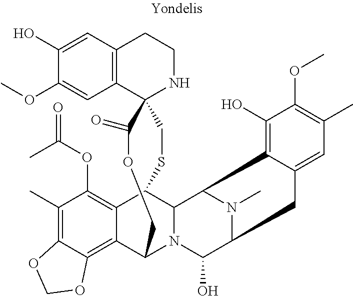 |
| Methylating Agents | Reactive N-methyl group | Methylate DNA | 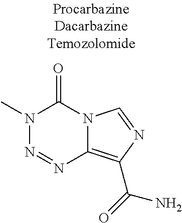 |
| Platinum | Pt(II) | Covalently binds | Cisplatin |
| compounds | | to DNA | Carboplatin |
| | | | Oxaliplatin |
| Nucleoside analogs | Resemble purine or | Incorporate into | Acyclovir |
| | pyrimidine bases | DNA during | Gemcitabine |
| | | replication | 5-fluorouracil |
| | | | Cytosine arabinoside |
| | | | Ganciclovir |
| Camptothecin derivatives | Quinoline alkaloids | Inhibits activity of topoisomerase I | 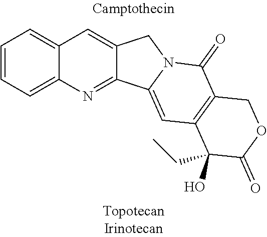 [SMILES: CC[C@@]1(O)C(=O)OCc2c1cc1n(c2=O)Cc2cc3ccccc3nc2-1] |
| Anthracycline derivatives | Anthracycline antibiotics | Inhibit activity of topoisomerase II | 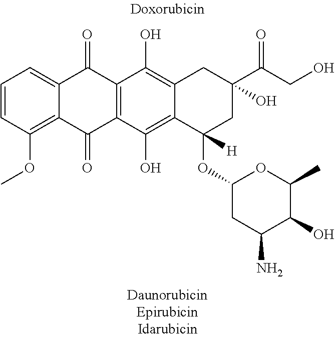 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1] |
| Epipodophyllotoxin | Epipodophyllotoxin | Inhibit activity of | Etoposide |
| derivatives | | topoisomerase II | Teniposide |
| Anthracenedione | Anthracenedione | Intercalate DNA | Mitoxantrone |
| derivatives | | | Pixantrone |

In a particular embodiment, the agent that interferes with DNA replication is a nitrogen mustard alkylating agent, or any intermediary or active metabolite thereof. Nitrogen mustards are non-specific DNA alkylating agents. Nitrogen mustards form cyclic aminium ions (aziridinium rings) by intramolecular displacement of the chloride by the amine nitrogen. This azidirium group is then capable of alkylating DNA by attacking the N-7 nucleophilic center on the guanine base. Upon displacement of the second chlorine, a second alkylation step occurs that results in the formation of interstrand cross-links (ICLs). These lesions are highly cytotoxic since they block fundamental metabolic processes such as DNA replication and transcription.

The methods of the invention encompass the use of any such non-specific nitrogen mustard DNA alkylating agents. Particularly suitable nitrogen mustard alkylating agents may include for example, and without limitation, cyclophosphamide, palifosfamide, bendamustine, and ifosfamide.

Ifosfamide is a nitrogen mustard alkylating agent. The IUPAC name for ifosfamide is N-3-bis(2-chloroethyl)-1,3,2-oxazaphosphinan-2-amide-2-oxide. Ifosfamide is commonly known as Ifex®. The chemical structure of ifosfamide is:

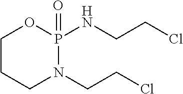 [SMILES: O=P1(NCCCl)OCCCN1CCCl]

Palifosfamide is an active metabolite of ifosfamide that is covalently linked to the amino acid lysine for stability. Palifosfamide irreversibly alkylates and cross-links DNA through GC base pairs, resulting in irreparable 7-atom inter-strand cross-links; inhibition of DNA replication and/or cell death. Palifosfamide is also known as Zymafos®.

Bendamustine is another nitrogen mustard alkylating agent. The IUPAC name for Bendamustine is 4-[5-[Bis(2-chloroethyl)amino]-1-methylbenzimidazol-2-yl]butanoic acid, and it is commonly referred to as Treakisym®, Ribomustin®, Levact® and Treanda®. The chemical structure of bendamustine is:

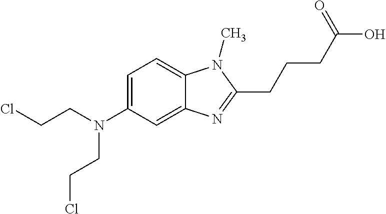 [SMILES: Cn1c(CCCC(=O)O)nc2cc(N(CCCl)CCCl)ccc21]

Also encompassed by the methods of the invention is the use of intermediary and/or active metabolites of DNA alkylating agents, and particularly intermediary and/or active metabolites of the nitrogen mustard DNA alkylating agents described herein. Such metabolites include, without limitation, aldophosphamide, 4-hydroxycyclophosphamide, 4-hydroxyifosfamide, chloracetaldehyde and phosphamide mustard.

In a further embodiment, the agent that interferes with DNA replication may be any suitable pharmaceutically acceptable salt, ester, tautomer, stereoisomer, racemic mixture, solvate, hydrate or prodrug of the alkylating agents, platinum compounds, methylating agents, or nucleoside analogs described herein.

In a particular embodiment, the agent that interferes with DNA replication for use in the methods of the invention is cyclophosphamide. Cyclophosphamide (N,N-bis(2-chloroethyl)-1,3,2-oxazaphosphinan-2-amine 2-oxide), also known as cytophosphane, is a nitrogen mustard alkylating agent. The chemical structure of cyclophosphamide is:

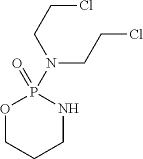 [SMILES: O=P1(N(CCCl)CCCl)NCCCO1]

Cyclophosphamide is also known and referred to under the trade-marks Endoxan®, Cytoxan®, Neosar®, Procytox® and Revimmune®. Other nitrogen mustard alkylating agents in the same class as cyclophosphamide include, without limitation, palifosfamide, bendamustine and ifosfamide.

Cyclophosphamide (CPA) is a prodrug which is typically administered via intravenous infusion, but also can be administered parenterally and orally (de Jonge, Huitema et al. 2005) with little difference in bioavailability (Duma, Rogers et al. 1979). CPA is converted to its active metabolites, 4-hydroxy-CPA and aldophosphamide, by oxidation by P450 enzymes in the liver (Emmenegger, Shaked et al. 2007; 2011). The active metabolites of CPA are lipid soluble and enter cells through passive diffusion. Intracellular 4-OH-CPA spontaneously decomposes into phosphoramide mustard which is the ultimate active metabolite. Phosphoramide mustard catalyzes intra- and interstrand DNA cross-links as well as DNA-protein cross-links that inhibit DNA replication leading to cell death (de Jonge, Huitema et al. 2005). Phosphoramide mustard is eliminated by enzymatic conversion to carboxyphoshphamide by cytoplasmic aldehyde dehydrogenase (ALDH) (Emmenegger, Shaked et al. 2007; 2011).

Cells with low levels of ALDH tend to accumulate CPA metabolites and are more sensitive to its effects, and indeed tumor upregulation of ALDH is one mechanism of CPA resistance (Zhang, Tian et al. 2005). Besides ALDH, low intracellular ATP levels have also been associated with CPA selectivity towards particular cells types (Zhao, Cao et al. 2010). At high doses, typically in the range of 1-5 g/m², the effects of CPA are most cytotoxic to rapidly dividing cells indiscriminate of cell type, and CPA is myelosuppressive since most hematogenic cells are rapidly dividing (Bruce, Meeker et al. 1966; Smith and Sladek 1985).

Total systemic clearance of CPA and its metabolites varies between 5-9 hours, and peak plasma levels of the parent also vary considerably between patients (3-11 hours) reflecting genetic differences in metabolism from person to person (Cohen, Jao et al. 1971; Mouridsen, Faber et al. 1974). Repeated administration of CPA is reported to shorten elimination half-life by increasing activity of enzymes involved in metabolism (D'Incalci, Bolis et al. 1979), but whether this leads to increased metabolism of the active metabolite is not known (de Jonge, Huitema et al. 2005), particularly at low doses (Emmenegger, Shaked et al. 2007).

Dose translation from human to murine studies is calculated using the following equation:

$$\underline{Human dose ( mg \text{/} kg )} = \underline{Animal Km}$$

$$Animal dose ( mg \text{/} kg ) = Human Km$$

Where the constant mouse Km value is 3 and human Km value is 37 (Reagan-Shaw, Nihal et al. 2008).

In the last two decades, low dose CPA has been appreciated for its immune modulatory and anti-angiogenic effects. In contrast to high dose CPA, low doses of CPA, typically 100-300 mg/m², lack widespread cytotoxic activity but do appear to enhance immune-mediated tumor elimination by selectively modulating cells of the immune system and also by reducing angiogenesis within the tumor microenvironment. The mechanisms of action and uses of low dose CPA are further described, for example, in WO2014/153636.

In an embodiment, the methods disclosed herein comprise administering an agent that interferes with DNA replication.

The agent that interferes with DNA replication is typically administered in an amount sufficient to provide an immune-modulating effect. As used herein, the expression “immune-modulating effect” refers to the ability of the agent that interferes with DNA replication to alter (modulate) one or more aspects of the immune system and/or cells of the immune system. In an embodiment, the “amount sufficient to provide an immune-modulating effect” is an amount of the agent that is capable of selectively affecting DNA replication in cells the immune system. For example, the amount of agent may be an amount sufficient to selectively target rapidly dividing cells of the immune system to cause programmed cell death.

The “amount sufficient to provide an immune-modulating effect” may interchangeably be referred to herein as a “low dose” amount. As relates to a particular embodiment of the invention where the agent that interferes with DNA replication is the alkylating agent cyclophosphamide, the expression “low dose” typically refers to a dose of cyclophosphamide that is less than or equal to 300 mg/m², such as for example 25-300 mg/m² and more particularly 100-300 mg/m². In an embodiment, the low dose amount of cyclophosphamide is 10, 25, 50, 75 or 100 mg BID (two times daily). In a particular embodiment, the low dose amount of cyclophosphamide is 50 mg BID. The “low dose” amounts of other agents that interfere with DNA replication, as encompassed herein, would be known to those skilled in the art, or could be determined by routine skill.

In a particular embodiment, the methods disclosed herein comprise a cycle of low dose metronomic cyclophosphamide. For purposes of the present disclosure, “metronomic” is meant to refer to a frequent administration of a lower than normal dose amount of the agent that interferes with DNA replication (e.g. cyclophosphamide). As used herein, the term “normal dose amount” may refer, for example and without limitation, to either: (i) the established maximum tolerated dose (MTD) or standard dose via a traditional dosing schedule, or (ii) in instances where a low dose single bolus amount has been established for a particular agent that interferes with DNA replication, than to that low dose amount.

In metronomic dosing, the same, lower, or higher cumulative dose over a certain time period as would be administered via a traditional dosing schedule may ultimately be administered. In a particularly suitable embodiment, this is achieved by extending the time frame during which the dosing is conducted and/or increasing the frequency of administrations, while decreasing the amount administered as compared to the normal dose amount. For example, where a low dose amount of 300 mg/m² of an agent that interferes with DNA replication is typically administered (e.g. by single bolus injection), a metronomic regimen may comprise administering the same amount over a period of several days by administering frequent low doses.

In an embodiment of the methods disclosed herein, metronomic treatment with the agent that interferes with DNA replication (e.g. cyclophosphamide) is intended to encompass a daily low dose administration of the agent over a certain period of time, such as for example a period of 2, 3, 4, 5, 6 or 7, or more, consecutive days. During these days of metronomic dosing, the agent that interferes with DNA replication may be provided at frequent regular intervals or varying intervals. For example, in an embodiment, a dose of the agent that interferes with DNA replication may be administered every 1, 2, 3, 4, 6, 8, 12 or 24 hours. In another embodiment, a dose of the agent that interferes with DNA replication may be administered once every 2, 3, or 4 days. In a particular embodiment, a dose of the agent that interferes with DNA replication may be administered two times daily.

In some embodiments, there may be breaks or gaps in the periods of metronomic treatment with the agent that interferes with DNA replication. In this manner, metronomic treatment may occur in a cyclic fashion, alternating between on and off periods of administration. Particularly suitable are intervals where the agent that interferes with DNA replication is administered to the subject daily on alternating weekly intervals. For instance, a one week period of administration of the agent that interferes with DNA replication is followed by a one week suspension of treatment, and the cycle repeats.

In an embodiment therefore, the methods disclosed herein comprise administering the agent that interferes with DNA replication to the subject daily for a period of 7 consecutive days, beginning every second week. In a particular aspect of this embodiment, the administration of the agent that interferes with DNA replication begins about 7 days prior to the first administration of the depot-forming vaccine. In a further aspect of this embodiment, the agent that interferes with DNA replication may be administered at a dose of 50 mg BID (two times daily) on each day of administration.

In an embodiment of the methods disclosed herein, the agent that interferes with DNA replication may be administered as a priming agent during the intermittent period between each administration of the depot-forming vaccine and/or non-depot-forming vaccine.

As the skilled person will appreciate, the frequency and duration of the administration of the agent that interferes with DNA replication, as well as the administration of the depot-forming and non-depot-forming vaccines, may be adjusted as desired for any given subject within the parameters described above. Factors that may be taken into account include, e.g.: the nature of the one or more antigens in the vaccine; the type of disease or disorder; the age, physical condition, body weight, sex and diet of the subject; and other factors.

The agent that interferes with DNA replication may be administered by any suitable delivery means and any suitable route of administration. In an embodiment, the agent that interferes with DNA replication is administered orally, such as in the form of a pill, tablet or capsule. In an alternate embodiment, the agent is administered by injection (e.g. intravenous). In a particular embodiment of the methods disclosed herein, the agent is cyclophosphamide and it is administered orally.

In a particular embodiment of the methods disclosed herein, the agent that interferes with DNA replication is cyclophosphamide.

Checkpoint Inhibitor

The methods disclosed herein may also comprise administering an immune response checkpoint inhibitor.

As used herein, an “immune response checkpoint inhibitor” refers to any compound or molecule that totally or partially reduces, inhibits, interferes with or modulates one or more checkpoint proteins. Checkpoint proteins regulate T-cell activation or function.

Numerous checkpoint proteins are known, such as for example CTLA-4 and its ligands CD80 and CD86; and PD-1 and its ligands PD-L1 and PD-L2. Checkpoint proteins are responsible for co-stimulatory or inhibitory interactions of T-cell responses. Checkpoint proteins regulate and maintain self-tolerance and the duration and amplitude of physiological immune responses. Herein, the term “immune response checkpoint inhibitor” may be used interchangeably with “checkpoint inhibitor”.

In some embodiments, the immune response checkpoint inhibitor is an inhibitor of Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, BTLA, CD2, CD27, CD28, CD30, CD40, CD70, CD80, CD86, CD160, CD226, CD276, DR3, GAL9, GITR, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGIT, VISTA, VTCN1, or any combination thereof.

In some embodiments, the immune response checkpoint inhibitor is an inhibitor of PD-L1, PD-1, CTLA-4 or any combination thereof.

In some embodiments, the immune response checkpoint inhibitor is an inhibitor of PD-L1 or PD-1. In an embodiment, the inhibitor of PD-L1 or PD-1 may be an anti-PD1 or anti-PDL1 antibody, such as for example and without limitation, those disclosed in WO 2015/103602. For example, in an embodiment, the anti-PD-1 antibody or anti-PD-L1 antibody may be selected from: nivolumab, pembrolizumab, pidilizumab, BMS-936559 (see ClinicalTrials.gov; Identifier NCT02028403), MPDL3280A (Roche, see ClinicalTrials.gov; Identifier NCT02008227), MDX1105-01 (Bristol Myers Squibb, see ClinicalTrials.gov; Identifier NCT00729664), MEDI4736 (MedImmune, see ClinicalTrials.gov; Identifier NCT01693562), and MK-3475 (Merck, see ClinicalTrials.gov; Identifier NCT02129556). In an embodiment, the anti-PD-1 antibody may be RMP1-4 or J43 (BioXCell) or a human or humanized counterpart thereof.

In some embodiments, the immune response checkpoint inhibitor is an inhibitor of CTLA-4. In an embodiment, the inhibitor of CTLA-4 may be an antibody, such as for example and without limitation, ipilimumab (Bristol-Myers Squibb) or BN13 (BioXCell). In another embodiment, the anti-CTLA-4 antibody may be UC10-4F10-11, 9D9 or 9H10 (BioXCell) or a human or humanized counterpart thereof.

The one or more immune response checkpoint inhibitors may be administered by any suitable route. In some embodiments, the route of administration of the one or more immune response checkpoint inhibitors is parenteral, mucosal, oral, sublingual, transdermal, topical, inhalation, intranasal, aerosol, intraperitoneal, intratumoral, intraocular, intratracheal, intrarectal, intragastric, vaginal, by gene gun, dermal patch or in eye drop or mouthwash form. In an embodiment, the immune response checkpoint inhibitor may be administered by subcutaneous injection.

As the skilled person will appreciate, the frequency and duration of the administration of the immune response checkpoint inhibitor may be adjusted as desired for any given subject. Factors that may be taken into account include, e.g.: the nature and type of the specific checkpoint inhibitor; the nature of the one or more antigens in the vaccine; the type of disease or disorder; the age, physical condition, body weight, sex and diet of the subject; and other factors.

In some embodiments, the one or more immune response checkpoint inhibitors may be administered before, after or concurrently with the depot-forming vaccine and/or non-depot-forming vaccine. In an embodiment, the immune response checkpoint inhibitor may be administered at a time subsequent to the first administration with the depot-forming vaccine. In aspects of this embodiment, the immune response checkpoint inhibitor may be administered at a time before or after the first administration of the non-depot-forming vaccine.

In an embodiment, administration of the immune response checkpoint inhibitor may begin on the same day as the first administration of the depot-forming vaccine and may be administered at a desired schedule thereafter. In an embodiment, the desired schedule may be administration of the immune response checkpoint inhibitor every 1, 2, 3, 4, 6, 8, 12 or 18 hours; every 1, 2, 3, 4, 5 or 6 days; or every 1, 2, 3 or 4 weeks. In an embodiment, the desired schedule may be once every 3 days.

There may be breaks or gaps in the periods of administration of the immune response checkpoint inhibitor. In this manner, administration may occur in a cyclic fashion, alternating between on and off periods of administration.

Vaccine Compositions

As used herein, the terms “vaccine”, “vaccine composition” or “composition” may be used interchangeably, as the context requires.

Vaccine compositions according to the invention may be administered to a subject in a therapeutically effect amount. As used herein, a “therapeutically effective amount” means an amount vaccine or active ingredient (e.g., one or more antigens) effective to stimulate, induce, maintain, boost or enhance an immune response in a subject, as applicable to either the depot-forming or non-depot-forming vaccines disclosed herein. In some embodiments, a therapeutically effective amount of the vaccine is an amount capable of inducing a clinical response in a subject in the treatment of a particular disease or disorder. Determination of a therapeutically effective amount of the vaccine is well within the capability of those skilled in the art, especially in light of the disclosure provided herein. The therapeutically effective amount may vary according to a variety of factors such as the subject's condition, weight, sex and age.

Vaccine compositions of the invention, for use in the methods and kits as described herein, are of two different types. The first type is a “depot-forming vaccine” and the second type is a “non-depot-forming vaccine”.

(i) Depot-Forming Vaccine

By “depot-forming vaccine”, it is meant that upon administration to the subject, the vaccine and its components (e.g. antigen, T-helper epitope, adjuvants, etc.) remain localized at the site of vaccine injection for a period of time, and are not rapidly dispersed throughout the body of the subject. This is referred to herein as a “depot effect”, whereby a substantial release of the antigen, or the antigen and one or more other vaccine components, from the site of injection does not occur for a prolonged period of time. The period of time by which the antigen or the antigen and one or more other vaccine components remain at the site of injection may be dependent upon how the depot effect is achieved. As used herein, the term “depot-forming vaccine” broadly means that a substantial proportion of the antigen or the antigen and one or more other vaccine components are held at the site of injection for a longer period of time than if the antigen and other components were administered in a non-depot-forming vaccine. Non-depot-forming vaccines are described later herein.

In an embodiment, the term “depot-forming vaccine” means that a substantial proportion (e.g. at least 50%, at least 60%, at least 70%, at least 80%, at least 90%, at least 95%, or 100%) of the antigen or the antigen and one or more other vaccine components remain localized at the site of injection for at least about 36 hours, 48 hours, 72 hours, 96 hours, 120 hours, 144 hours, 168 hours, or 192 hours. In an embodiment, less than 1%, 2%, 3%, 4% or 5% of the antigen or the antigen and one or more other vaccine components are released from the site of injection within 24 hours of administration of the depot-forming vaccine. In an embodiment, at least 80% of the antigen or the antigen and one or more other vaccine components remain localized at the site of injection for at least about 48 hours after administration. In an embodiment, at least 60% of the antigen or the antigen and one or more other vaccine components remain localized at the site of injection for at least about 72 hours after administration.

The release kinetics of a vaccine composition can be determined by various means known in the art. As an example, to mimic physiological conditions, a sample of a vaccine composition (e.g. 500 μl) can be added to a vial of phosphate buffer (e.g. 1 ml; 10 mM at pH 7.4). The vials can be kept at 37° C. and tested at appropriate intervals by removing aliquots of the aqueous layer and analyzing for the presence of the vaccine components (e.g. by HPLC) in the aqueous environment.

A depot-forming vaccine may be prepared by formulating the vaccine components in a hydrophobic carrier, such as for example oil. The continuous hydrophobic phase of such a carrier (e.g. oil) would be immiscible with the aqueous environment of a vaccinated subject, resulting in the depot effect at the site of injection. Embodiments of how vaccine components may be formulated in a hydrophobic carrier are described below. It should be understood that the description of how antigens may be made miscible in a hydrophobic carrier may also be applicable to, and may be used for, other vaccine components as well, such as for example T-helper epitopes, adjuvants, etc.

(ii) Formulating Vaccine Components in a Hydrophobic Carrier

The ability to solubilize antigens and other vaccine components in formulations having a hydrophobic carrier such as oil, to the exclusion of water, has several benefits over conventional aqueous systems and other systems in which antigens are suspended in a hydrophilic phase.

Formulating antigens in a water-free environment increases their stability with respect to hydrolysis, thermal denaturation, and light sensitivity. With regards to vaccine development, the holding of antigens in a hydrophobic carrier decreases their tendency to quickly disperse at the site of injection, and forces the immune system to actively uptake the vaccine components, thus facilitating their immunogenicity. Under typical conditions, hydrophilic antigens will not dissolve in a hydrophobic carrier based on the high surface tension that exists between the two. However, there are several ways to facilitate the incorporation of antigens into a fully hydrophobic environment, such as an oil formulation.

Thus, in an embodiment, the one or more antigens of the depot-forming vaccine disclosed herein are sufficiently hydrophobic, or are made sufficiently hydrophobic, such that the one or more antigens are miscible in the hydrophobic carrier.

As used herein, by “sufficiently hydrophobic” it is meant that the antigen is compatible with the hydrophobic carrier. Compatibility may be determined by various means, but generally an antigen is compatible with a hydrophobic carrier if it is miscible in the hydrophobic carrier. In the context herein, “miscible” means that the antigen is capable of being resuspended, dissolved, or otherwise distributed in the hydrophobic carrier. Although typically a homogeneous solution will result, it is not necessary for the purpose of the present disclosure that a homogeneous solution be formed, so long as the antigen resuspends, dissolves or otherwise distributes into the hydrophobic carrier. Also, for the present disclosure and depending on the antigen/hydrophobic carrier combination, it is possible that a small fraction (e.g. less than 1%, 2%, 3%, 4%, 5%, 6%, 7%, 8%, 9% or 10%) of the antigen may not resuspend or dissolve, but the antigen may still be considered miscible.

There are various means known in the art by which miscibility can be determined. Generally, miscibility is determined optically. When the antigen is miscible in a hydrophobic carrier, the resulting liquid is clear. Clarity can be measured by the naked eye or by instrumentation. If there is no cloudiness or particles visible to the naked eye, then the antigen is considered miscible in the hydrophobic carrier. As the skilled person will appreciate, miscibility may vary with concentration of the antigen and the higher the concentration of antigen, the greater the likelihood of cloudiness or the presence of visible particles. Therefore, in some embodiments, the term “miscible” as used herein means that a therapeutically effective amount or concentration of the antigen is capable of being resuspended, dissolved, or otherwise distributed in the hydrophobic carrier.

In a first embodiment, an antigen of interest may be naturally hydrophobic, making direct dissolution in a hydrophobic carrier, such as oil, possible. Without limitation, some of the most common antigens that display this characteristic are those that construct specific molecule binding sites and pockets or make up transmembrane domains of proteins. Signal peptides and transmembrane domains have been found to be rich in hydrophobic CD8+ T cell epitopes, which have the ability to bind multiple MHC alleles due to their hydrophobic nature and sequences (Kovjazin et al, Mol Immunol 2011 48(8):1009). Antigens containing a high proportion of hydrophobic amino acid residues are generally under-represented in vaccine design, due to their formulation issues in standard aqueous vaccine formulations. The vaccine formulations disclosed herein may comprise one or more antigens that are naturally hydrophobic.

In a second embodiment, the hydrophobicity of antigens may be increased by modification to the antigen itself. Without limitation, an example of such modification is protein lipidation, resulting in lipopeptides. Methods of protein lipidation include, but are not limited to, N-terminal myristoylation (Resh et al, Biochim Biophys Acta 1999 1451:1), attachment of cholesterol to the C-terminus (Karpen et al, J Biol Chem 2001 276:19503), S-prenylation of cysteine residues at or close to the C-terminus (Zhang et al, Annu Rev Biochem 1996 65:241) and S-palmitoylation of cysteines throughout the protein (Smotrys et al, Annu Rev Biochem 2004 73:559). These proteins will often take on the increased hydrophobicity of the lipid moieties. More recently, the option to attach lipids with adjuvant activities has been explored, such as Pam2Cys/Pam3Cys (Moyle et al, Current Medicinal Chemistry 2008 15(5):506), palmitic acid (Robinson et al, Immunology 1992 76(4):593), and other lipoamino acids (Hayman et al, Immunol Cell Biology 2002 80:178). The vaccine formulations disclosed herein may comprise one or more antigens that are modified by lipidation.

Antigens can also be non-covalently complexed to hydrophobic molecules, compounds or complexes using hydrophobic ion-pairing in order to increase the miscibility of the antigen in the hydrophobic carrier. Briefly, hydrophobic ion-pairing is a method for increasing the solubility of an antigen in a hydrophobic carrier by replacing the counter ions of the antigen with a charged organic carrier molecule. In EP1150710B1, immunogenic complexes were formed via an electrostatic association between a positively charged antigen and a negatively charged organic carrier molecule, such as a saponin or saponin complex. This approach is sometimes favored over covalent bonding to hydrophobic molecules due to its simplicity and lower requirement for materials. It is sometimes used to enhance the loading and stability of proteins within the hydrophobic compartment of nanoparticles (Yoo et al, Journal of Pharmaceutical Science 2001 90(2):194). The vaccine formulations disclosed herein may comprise one or more antigens that are non-covalently or covalently complexed to a hydrophobic molecule, compound or complex.

Although the above techniques have been successfully used to increase the hydrophobicity of antigens, there are very few examples in which these types of antigens are dissolved directly in a hydrophobic carrier such as oil, to the exclusion of water. Typical formulations containing an antigen and hydrophobic carrier (e.g. oil) are also comprised of a hydrophilic phase. For instance, there are many examples of water-in-oil and oil-in-water emulsions, in which an antigen is suspended in an aqueous phase and emulsified with oil. However, emulsions may become unstable once injected in vivo, causing the separation of the aqueous and oily phases of the composition. This leads to premature or accelerated release of antigens and other components. In other instances, the presence of antigens in the aqueous compartment or an oil-in-water emulsion is expected to dissipate from the site of injection relatively quickly, typically in less than 48 hours. Antigens which may be contained within microscopic oil droplets of an oil-in-water emulsion can also be removed from the site of injection and do not persist.

Liposomes have been used in emulsions as a vesicle to encapsulate antigens as well as an emulsifier to stabilize the formulation. Hydrophilic antigens are typically entrapped in the aqueous interior, while hydrophobic antigens can be intercalated in the lipid bilayer or dispersed in the oil phase. As with other emulsions, liposome containing emulsions can be cumbersome to prepare and can separate into aqueous and oil phases in vivo.

In a third embodiment, an antigen may be made sufficiently hydrophobic by using an amphiphile. In a particularly suitable embodiment, an antigen may be solubilized with one or more amphiphiles in a hydrophobic carrier in the absence of a hydrophilic phase.

An “amphiphile” is a compound having both hydrophilic and hydrophobic (lipophilic) parts or characteristics. The term “amphiphile” may be used interchangeably with “amphiphilic” and “amphipathic”. In some embodiments, suitable amphiphiles also include emulsifiers such as those described herein below. Exemplary embodiments of emulsifiers that are encompassed herein by the term “amphiphile” include, without limitation, polysorbates (e.g. sorbitan monooleate), mannide oleate (Arlacel™ A), lecithin, Tween™ 80, and Spans™ 20, 80, 83 and 85. The amphiphile can facilitate the incorporation of vaccine components with hydrophilic affinity into a hydrophobic carrier such as an oil. The vaccine components can include, without limitation, antigens and/or adjuvants and/or other ingredients (e.g. T-helper epitopes) that can facilitate the production of an immune response.

Without limitation, the hydrophobic portion of an amphiphile is typically a large hydrocarbon moiety, such as a long chain of the form CH₃(CH₂)ₙ, with n>4. The hydrophilic portion of an amphiphile is usually either a charged group or a polar uncharged group. Charged groups include anionic and cationic groups. Examples of anionic charged groups include the following (wherein the hydrophobic part of the molecule is represented by “R”): carboxylates: RCO₂⁻; sulfates: RSO₄⁻; sulfonates: RSO₃⁻; and phosphates (the charged functionality in phospholipids). Cationic charged groups include e.g. amines: RNH₃⁺ (“R” again representing the hydrophobic part of the molecule). Uncharged polar groups include e.g. alcohols with large R groups, such as diacyl glycerol (DAG). Amphiphiles may have several hydrophobic parts, several hydrophilic parts, or several of both. Proteins and some block copolymers are examples. Steroids, cholesterol, fatty acids, bile acids, and saponins, are also amphiphiles.

There are numerous amphiphiles which may be used and the vaccine formulations disclosed herein may contain a single type of amphiphile or a mixture of different types of amphiphiles.

In an embodiment, the amphiphile is a lipid. Although any amphiphilic lipid may be used, particularly suitable lipids may include those with at least one fatty acid chain containing at least 4 carbons, and typically about 4 to 28 carbons in length. The fatty acid chain may contain any number of saturated and/or unsaturated bonds. The lipid may be a natural lipid or a synthetic lipid. Non-limiting examples of amphiphilic lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids.

In an embodiment, the amphiphile is a phospholipid or a mixture of phospholipids. Broadly defined, a “phospholipid” is a member of a group of lipid compounds that yield on hydrolysis phosphoric acid, an alcohol, fatty acid, and nitrogenous base.

Phospholipids that may be used in the preparation of liposomes include for example, and without limitation, those with at least one head group selected from the group consisting of phosphoglycerol, phosphoethanolamine, phosphoserine, phosphocholine (e.g. DOPC; 1,2-Dioleoyl-sn-glycero-3-phosphocholine) and phosphoinositol. In some embodiments, a mixture of DOPC and unesterified cholesterol may be used. When unesterified cholesterol is used, the cholesterol may be used in an amount equivalent to about 10% of the weight of phospholipid (e.g. in a DOPC:cholesterol ratio of 10:1 w/w). For example, the amount of DOPC in a single dose of a composition as described herein may be 120 mg/ml and the amount of cholesterol may be 12 mg/ml. In certain embodiments, the amount of DOPC may be about 30 mg/unit dose of the composition and the amount of cholesterol may be about 3 mg/unit dose. The cholesterol is used to stabilize the formation of phospholipid vesicles. If a compound other than cholesterol is used, one skilled in the art can readily determine the amount needed

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs or sheep's wool.

All of these and other phospholipids may be used in the practice of the invention. Phospholipids can be purchased, for example, from Avanti lipids (Alabastar, Ala., USA), and lipoid LLC (Newark, N.J., USA).

Antigens may be made sufficiently hydrophobic to be miscible in the hydrophobic carrier by the presence of one or more amphiphiles. Exemplary disclosures of the preparation of vaccine and immunogenic compositions comprising antigens, amphiphiles and a hydrophobic carrier include e.g. WO1996/014871 and WO2009/043165.

In an embodiment, the amphiphile may be substantially evenly dispersed in the hydrophobic carrier, whereby the presence of the amphiphile alone is sufficient to make the antigen miscible in the hydrophobic carrier.

In another embodiment, the amphiphile may be closely associated with the antigen so as to make the antigen miscible in the hydrophobic carrier. By “closely associated”, it is meant that the amphiphile is in such proximity with the antigen that the antigen is presented in a form that it is miscible in the hydrophobic carrier. The close association may or may not involve physical interaction between the antigen and the amphiphile. Typically, the hydrophilic part of the amphiphile is oriented towards the hydrophilic moieties on the antigen. The amphiphiles may remain substantially separate from one another or they may form various different types of structures, assemblies or arrays.

Exemplary embodiments of the types of structures, assemblies or arrays that the amphiphiles may form include, without limitation: single layer sheets, bilayer sheets, multilayer sheets, single layer vesicular structures (e.g. micelles), bilayer vesicular structures (e.g. unilamellar or multilamellar vesicles), or various combinations thereof. By “single layer” it is meant that the amphiphiles do not form a bilayer, but rather remain in a layer with the hydrophobic part oriented on one side and the hydrophilic part oriented on the opposition side. By “bilayer” it is meant that the amphiphiles form a two-layered sheet, typically with the hydrophobic part of each layer internally oriented toward the center of the bilayer with the hydrophilic part externally oriented. However, the opposite configuration is also possible. The term “multilayer” is meant to encompass any combination of single and bilayer structures. The form adopted may depend upon the specific antigen, the specific amphiphile, and/or the specific hydrophobic carrier that is used.

In an embodiment, the structure, assembly or array formed by the amphiphile may partially or completely surround the antigen. As an example, the amphiphile may form a closed vesicular structure around the antigen.

In an embodiment, the vesicular structure is a single layer vesicular structure. An example of such a structure is a micelle. A typical micelle in aqueous solution forms an aggregate with the hydrophilic parts in contact with the surrounding aqueous solution, sequestering the hydrophobic parts in the micelle center. In contrast, in a hydrophobic carrier, an inverse/reverse micelle forms with the hydrophobic parts in contact with the surrounding aqueous solution, sequestering the hydrophilic parts in the micelle center. A spherical reverse micelle can package an antigen with hydrophilic affinity within its core.

In an embodiment, the vesicular structure is a micelle or an inverse/reverse micelle. Without limitation, the size of the micelles or inverse/reverse micelles range from 2 nm (20 A) to 20 nm (200 A) in diameter. In a particular embodiment, the size of the micelles or inverse/reverse micelles is about 10 nm in diameter.

In another embodiment, the vesicular structure is a bilayer vesicular structure, such as for example, a liposome. Liposomes are completely closed lipid bilayer membranes containing an entrapped aqueous volume. Liposomes may be unilamellar vesicles (possessing a single bilayer membrane) or multilamellar vesicles characterized by multimembrane bilayers, each bilayer may or may not be separated from the next by an aqueous layer. A general discussion of liposomes can be found in Gregoriadis G. Immunol. Today, 11:89-97, 1990; and Frezard, F., Braz. J. Med. Bio. Res., 32:181-189, 1999. Liposomes can adsorb to virtually any type of cell and then release an incorporated agent (e.g. antigen). Alternatively, the liposome can fuse with the target cell, whereby the contents of the liposome empty into the target cell. Alternatively, a liposome may be endocytosed by cells that are phagocytic.

Liposomes have been used in the preparation of compositions comprising a hydrophobic carrier as a vesicle to encapsulate antigens as well as an emulsifier to stabilize the formulation (see e.g. WO2002/038175, WO2007/041832, WO2009/039628, WO2009/146523 and WO2013/049941. Hydrophilic antigens are typically entrapped in the aqueous interior, while hydrophobic antigens can be intercalated in the lipid bilayer or dispersed in the oil phase.

Other embodiments of bilayer and mutilayer vesicular structures include, without limitation: niosomes, transfersomes, virosomes, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these vesicular structures are well known in the art (see e.g. Kreuter, J., ed., Colloidal Drug Delivery Systems, vol. 66, Marcel Dekker, Inc., 1994).

The approaches and vaccine compositions described above are exemplary non-limiting embodiments of how vaccine components may be formulated in a hydrophobic carrier. Again, it is to be understood that the above description of how antigens may be made miscible in a hydrophobic carrier may also be applicable to, and may be used for, other vaccine components such as, for example, T-helper epitopes, adjuvants, etc.

The volume per dose of the depot-forming vaccine as described herein may vary depending on characteristics of the subject (e.g. size, weight, age, sex, etc), the type of hydrophobic carrier used, and other factors. One skilled in the art will be able to determine, without undue experimentation, the appropriate volume. In an embodiment, the dose volume will be about 0.01 to 1 ml/dose. In certain embodiments, the dose volume may be about 50 μl. In certain embodiments, such as for example for human subjects, the dose volume may be about 250 μl. In such embodiments, the hydrophobic carrier may for example be Montanide ISA51 VG.

(iii) Non-Depot-Forming Vaccine

By “non-depot-forming vaccine”, it is meant that upon administration to the subject, the vaccine and its components (e.g. antigen, T-helper epitope, adjuvants, etc.) are rapidly dispersed in the body of the subject and a “depot effect” is not obtained or does not persist. For a non-depot-forming vaccine, the antigen or the antigen and one or more other vaccine components are cleared from the site of injection substantially faster than the respective components of a depot-forming vaccine. Thus, the non-depot-forming vaccine rapidly re-exposes a previously primed immune system to the antigens and other vaccine components, and then dissipates from the site of injection before an adverse reaction can be generated at the site. In an embodiment, greater than 50%, 60%, 70%, 80%, 90% or 100% of the antigen or the antigen and one or more other vaccine components of a non-depot-forming vaccine have been cleared from the site of injection with about 3 hours, 6 hours, 12 hours, 18 hours, 24 hours, 36 hours, or 48 hours.

The non-depot-forming vaccine may by any vaccine composition, sharing the same antigenic determinant (i.e. antigen or epitope) as the depot-forming vaccine, which does not result in a depot effect at the site of injection. In this regard, the vaccine components may formulated in any number of known pharmaceutically acceptable carriers. Generally, the carrier is miscible with the aqueous environment (e.g. bodily fluids, tissue, etc.) of a vaccinated subject.

As used herein, the term “pharmaceutically acceptable carrier” refers to a carrier that is ‘acceptable’ in the sense of being compatible with the other ingredients of a composition and not deleterious (e.g. toxic) to the recipient thereof. Typcially, the pharmaceutically acceptable carrier is a medium that does not interfere with the immunomodulatory activity of the active ingredients.

Some examples of pharmaceutically acceptable carriers that may be used in a non-depot-forming vaccine include, but are by no means limited to, e.g., water, oil-in-water emulsions, phosphate buffered saline, glycerol, ethanol, Ringer's solution, dextrose solution, serum-containing solutions, Hank's solution, and other aqueous physiologically balanced solutions. See, for example, Remington: The Science and Practice of Pharmacy, 2000, Gennaro, A R ed., Eaton, Pa.: Mack Publishing Co.

In an embodiment, a non-depot-forming vaccine may be prepared by formulating the vaccine components in an aqueous carrier, such as water or PBS. When a non-depot-forming vaccine is introduced into a subject (an aqueous environment), antigen particles and other vaccine components suspended in an aqueous carrier are rapidly dispersed in the body of the subject. This is because the aqueous carrier of the vaccine is miscible with the aqueous environment (e.g. bodily fluids, tissue, etc.) in the body of the subject. This allows for fast trafficking and rapid accumulation of the vaccine constituents (e.g. antigen) to the draining lymph nodes.

In an embodiment, the carrier for the non-depot-forming vaccine is water.

The volume per dose of the non-depot-forming vaccine as described herein may vary depending on characteristics of the subject (e.g. size, weight, age, sex, etc), the type of carrier used, and other factors. One skilled in the art will be able to determine, without undue experimentation, the appropriate volume. In an embodiment, the dose volume will be about 0.01 to 1 ml/dose. In certain embodiments, the dose volume may be about 50 μl. In certain embodiments, such as for example for human subjects, the dose volume may be about 500 μl. In such embodiments, the carrier may sterile water.

The vaccine compositions (depot and non-depot-forming) as described herein may be administered by any means known in the art. In an embodiment, the vaccine compositions are administered by subcutaneous injection.

Antigens

The vaccine compositions disclosed herein, including both the depot-forming and non-depot-forming vaccines, may comprise one or more antigens.

As used herein, the term “antigen” refers to any substance or molecule that can bind specifically to components of the immune system. In some embodiments, suitable antigens of the compositions herein are those that are capable of inducing or generating an immune response in a subject. An antigen that is capable of inducing an immune response is said to be immunogenic, and may also be called an immunogen. Thus, as used herein, the term “antigen” includes immunogens and the terms may be used interchangeably unless specifically stated otherwise. The term antigen, as used herein, also includes haptens. As is understood in the art, a hapten is a small molecule that is antigenic (e.g. capable of being bound by components of the immune system), but is not immunogenic unless it is attached to a carrier molecule of some sort which supplies the immunogenicity.

Antigens that may be useful in the compositions of the invention include, for example and without limitation, a polypeptide, carbohydrate, a microorganism or a part thereof, such as a live, attenuated, inactivated or killed bacterium, virus or protozoan, or part thereof. The antigen may be, for example, a pathogenic biological agent, a toxin, an allergen, a peptide, a suitable native, non-native, recombinant or denatured protein or polypeptide, or a fragment thereof, or an epitope that is capable of inducing or potentiating an immune response in a subject. In some embodiments, the antigen may be one that is derived from an animal (an animal antigen), such as for example a human (a human antigen), or an antigen that is substantially related thereto.

As used herein, the term “derived from” encompasses, without limitation: an antigen that is isolated or obtained directly from an originating source (e.g. a subject); a synthetic or recombinantly generated antigen that is identical or substantially related to an antigen from an originating source; or an antigen which is made from an antigen of an originating source or a fragment thereof. The term “substantially related”, as this context, means that the antigen may have been modified by chemical, physical or other means (e.g. sequence modification), but that the resultant product remains capable of generating an immune response to the original antigen or to the disease or disorder associated with the original antigen.

As used herein, the term “antigen” also includes a polynucleotide that encodes a polypeptide that functions as an antigen. Nucleic acid-based vaccination strategies are known, wherein a vaccine composition that contains a polynucleotide is administered to a subject. The antigenic polypeptide encoded by the polynucleotide is expressed in the subject, such that the antigenic polypeptide is ultimately present in the subject, just as if the vaccine composition itself had contained the polypeptide. For the purposes of the present disclosure, the term “antigen”, where the context dictates, encompasses such polynucleotides that encode the polypeptide which functions as the antigen.

In some embodiments, the antigen is a molecule comprising at least one B cell epitope or CTL epitope, as defined below, and which, when suitably administered to a subject, induces or potentiates a humoral and/or cell-mediated immune response which is protective against the disease.

In some embodiments, the antigen may be one that is associated with cancer, an infectious disease, or an addiction disease.

Viruses, or parts thereof, that may be useful as antigens in the compositions herein include for example, and without limitation, Cowpoxvirus, Vaccinia virus, Pseudocowpox virus, herpes virus, Human herpesvirus 1, Human herpesvirus 2, Cytomegalovirus, Human adenovirus A-F, Polyomavirus, human papillomavirus (HPV), Parvovirus, Hepatitis A virus, Hepatitis B virus, Hepatitis C virus, human immunodeficiency virus (HIV), Orthoreovirus, Rotavirus, Ebola virus, parainfluenza virus, influenza virus (e.g. H5N1 influenza virus, influenza A virus, influenza B virus, influenza C virus), Measles virus, Mumps virus, Rubella virus, Pneumovirus, respiratory syncytial virus, human respiratory syncytial virus, Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus, Zika virus and varicella.

In an embodiment, a composition disclosed herein comprises an antigen that may potentially be useful for treating and/or preventing an influenza virus infection in a subject in need thereof. Influenza is a single-stranded RNA virus of the family Orthomyxoviridae and is often characterized based on two large glycoproteins on the outside of the viral particle, hemagglutinin (HA) and neuraminidase (NA). Numerous HA subtypes of influenza A have been identified (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York). In some embodiments, the antigen may be derived from the HA or NA glycoproteins.

In another embodiment, a composition disclosed herein comprises an antigen that may potentially be useful for treating and/or preventing an Ebola virus infection in a subject in need thereof.

In another embodiment, a composition disclosed herein comprises an antigen that may potentially be useful for treating and/or preventing a human papillomavirus (HPV) infection in a subject in need thereof. In more particular embodiments, a composition disclosed herein comprises an antigen that may potentially be useful for treating and/or preventing a HPV-related cervical cancer or HPV-related head and neck cancer. In some embodiments, the antigen is a peptide comprising the sequence RAHYNIVTF (HPV16E7 (H-2Db) peptide 49-57; R9F; SEQ ID NO: 3).

In another embodiment, a composition disclosed herein comprises an antigen that may potentially be useful for treating and/or preventing a respiratory syncytial virus (RSV) infection in a subject in need thereof. In more particular embodiments, a composition disclosed herein comprises an antigen that may potentially be useful for treating and/or preventing a lung disease associated with a RSV infection. In some embodiments, the antigen is derived from the ectodomain of the small hydrophobic protein as disclosed, for example, in WO2012/065997.

Bacteria or parts thereof that may be useful as antigens in the compositions herein include for example, and without limitation, Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

In an embodiment, a composition disclosed herein comprises an antigen that may potentially be useful for treating and/or preventing a Bacillus anthracis infection (i.e. Anthrax) in a subject in need thereof. Without limitation, the antigen contained in the vaccine may for example be anthrax recombinant protective antigen (rPA) (List Biological Laboratories, Inc.; Campbell, Calif.) or anthrax mutant recombinant protective antigen (mrPA) (Pfenex, Inc.; San Diego, Calif.).

Protozoa or parts thereof that may be useful as antigens in the compositions herein include for example, and without limitation, the genus Plasmodium (Plasmodium falciparum, Plasmodium malariae, Plasmodium vivax, Plasmodium ovale or Plasmodium knowlesi), which causes malaria.

In an embodiment, a composition disclosed herein comprises an antigen that may potentially be useful for treating and/or preventing a Plasmodium malariae infection (i.e. malaria) in a subject in need thereof.

The antigen may alternatively be a naturally occurring or synthesized toxin or allergen. A “toxin”, as used herein, refers to any substance produced by living cells or organisms (e.g. plants, animals, microorganisms, etc.) that is capable of causing a disease or ailment, or an infectious substance, or a recombinant or synthesized molecule capable of adverse effect. Toxins may be for example small molecules, peptides, or proteins. Toxins include drug substances such as, for example, cocaine. The toxin may be capable of being neutralized by an antibody. In such embodiments, the antigen may elicit the production of antibodies that bind to and sequester the toxin in circulation (e.g. the blood), thereby potentially preventing its delivery to another area of the body (e.g. the brain).

An “allergen”, as used herein, refers to any substance that can cause an allergy. The allergen may be derived from, without limitation, cells, cell extracts, proteins, polypeptides, peptides, polysaccharides, polysaccharide conjugates, peptide and non-peptide mimics of polysaccharides and other molecules, small molecules, lipids, glycolipids, and carbohydrates of plants, animals, fungi, insects, food, drugs, dust, and mites. Allergens include but are not limited to environmental aeroallergens; plant pollens (e.g. ragweed/hayfever); weed pollen allergens; grass pollen allergens; Johnson grass; tree pollen allergens; ryegrass; arachnid allergens (e.g. house dust mite allergens); storage mite allergens; Japanese cedar pollen/hay fever; mold/fungal spore allergens; animal allergens (e.g., dog, guinea pig, hamster, gerbil, rat, mouse, etc., allergens); food allergens (e.g. crustaceans; nuts; citrus fruits; flour; coffee); insect allergens (e.g. fleas, cockroach); venoms: (Hymenoptera, yellow jacket, honey bee, wasp, hornet, fire ant); bacterial allergens (e.g. streptococcal antigens; parasite allergens such as Ascaris antigen); viral antigens; drug allergens (e.g. penicillin); hormones (e.g. insulin); enzymes (e.g. streptokinase); and drugs or chemicals capable of acting as incomplete antigens or haptens (e.g. the acid anhydrides and the isocyanates).

Where a hapten is used in a composition of the invention, it may be attached to a carrier, such as for example a protein, to form a hapten-carrier adduct. The hapten-carrier adduct is capable of eliciting an immune response, whereas the hapten itself would not typically elicit a response. Non-limiting examples of haptens are aniline, urushiol (a toxin in poison ivy), hydralazine, fluorescein, biotin, digoxigenin and dinitrophenol.

In another embodiment, the antigen may be an antigen associated with a disease where it is desirable to sequester the antigen in circulation, such as for example an amyloid protein (e.g. Alzheimer's disease). Thus, in some embodiments, a composition of the invention comprises an antigen that may potentially be useful in the treatment and/or prevention of a neurodegenerative disease in a subject in need thereof, wherein the neurodegenerative disease is associated with the expression of the antigen.

In another embodiment, the antigen may be any one or more of the antigens disclosed in WO 2007/041832, such as for example the peptide antigens disclosed in Table 1 at pages 17-19 of WO 2007/041832.

For example, and without limitation, polypeptides or fragments thereof that may be useful as antigens in the compositions herein include those derived from Cholera toxoid, tetanus toxoid, diphtheria toxoid, hepatitis B surface antigen, hemagglutinin (e.g. H5N1 recombinant hemagglutinin protein), anthrax recombinant protective antigen (List Biological Laboratories, Inc.; Campbell, Calif.), anthrax mutant recombinant protective antigen (Pfenex, Inc.; San Diego, Calif.), neuraminidase, influenza M protein, PfHRP2, pLDH, aldolase, MSP1, MSP2, AMA1,Der-p-1, Der-f-1, Adipophilin, AFP, AIM-2, ART-4, BAGE, α-feto protein, BCL-2, Bcr-Abl, BING-4, CEA, CPSF, CT, cyclin D1Ep-CAM, EphA2, EphA3, ELF-2, FGF-5, G250, Gonadotropin Releasing Hormone (GNRH), HER-2, intestinal carboxyl esterase (iCE), IL13Ra2, MAGE-1, MAGE-2, MAGE-3, MART-1, MART-2, M-CSF, MDM-2, MMP-2, MUC-1, NY-EOS-1, MUM-1, MUM-2, MUM-3, pertussis toxoid protein, p53, PBF, PRAME, PSA, PSMA, RAGE-1, RNF43, RU1, RU2AS, SART-1, SART-2, SART-3, SAGE-1, SCRN 1, SOX2, SOX10, STEAP1, survivin, Telomerase, TGFβRII, TRAG-3, TRP-1, TRP-2, TERT and WT1.

The term “polypeptide” encompasses any chain of amino acids, regardless of length (e.g., at least 6, 8, 10, 12, 14, 16, 18, or 20 amino acids) or post-translational modification (e.g., glycosylation or phosphorylation), and includes, for example, natural proteins, synthetic or recombinant polypeptides and peptides, epitopes, hybrid molecules, variants, homologs, analogs, peptoids, peptidomimetics, etc. A variant or derivative therefore includes deletions, including truncations and fragments; insertions and additions, for example conservative substitutions, site-directed mutants and allelic variants; and modifications, including peptoids having one or more non-amino acyl groups (for example, sugar, lipid, etc.) covalently linked to the peptide and post-translational modifications. As used herein, the term “conserved amino acid substitutions” or “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Specific, non-limiting examples of a conservative substitution include the following examples:

| Original Residue | Conservative Substitution |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Val | Ile, Leu |

Polypeptides or peptides that have substantial identity to an antigen sequence may be used. Two sequences are considered to have substantial identity if, when optimally aligned (with gaps permitted), they share at least approximately 50% sequence identity, or if the sequences share defined functional motifs. In alternative embodiments, optimally aligned sequences may be considered to be substantially identical (i.e., to have substantial identity) if they share at least 60%, 70%, 75%, 80%, 85%, 90%, 95%, 96%, 97%, 98%, 99% identity over a specified region. The term “identity” refers to sequence similarity between two polypeptides molecules. Identity can be determined by comparing each position in the aligned sequences. A degree of identity between amino acid sequences is a function of the number of identical or matching amino acids at positions shared by the sequences, for example, over a specified region. Optimal alignment of sequences for comparisons of identity may be conducted using a variety of algorithms, as are known in the art, including the ClustalW program, available at http://clustalw.genome.ad.jp, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). For example, the “BLAST 2 Sequences” tool, available through the National Center for Biotechnology Information (through the internet at http://www.ncbi.nlm.nih.gov/BLAST/b12seq/wblast2.cgi) may be used, selecting the “blastp” program at the following default settings: expect threshold 10; word size 3; matrix BLOSUM 62; gap costs existence 11, extension 1. In another embodiment, the person skilled in the art can readily and properly align any given sequence and deduce sequence identity and/or homology by mere visual inspection.

Polypeptides and peptides used to practice the invention can be isolated from natural sources, be synthetic, or be recombinantly generated polypeptides. Peptides and proteins can be recombinantly expressed in vitro or in vivo. The peptides and polypeptides used to practice the invention can be made and isolated using any method known in the art. Polypeptide and peptides used to practice the invention can also be synthesized, whole or in part, using chemical methods well known in the art. See e.g., Caruthers (1980) Nucleic Acids Res. Symp. Ser. 215-223; Hom (1980) Nucleic Acids Res. Symp. Ser. 225-232; Banga, A. K, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

In some embodiments, the antigen may be a purified antigen, e.g., from about 25% to 50% pure, from about 50% to about 75% pure, from about 75% to about 85% pure, from about 85% to about 90% pure, from about 90% to about 95% pure, from about 95% to about 98% pure, from about 98% to about 99% pure, or greater than 99% pure.

As noted above, the term “antigen” also includes a polynucleotide that encodes the polypeptide that functions as an antigen. As used herein, the term “polynucleotide” encompasses a chain of nucleotides of any length (e.g. 9, 12, 18, 24, 30, 60, 150, 300, 600, 1500 or more nucleotides) or number of strands (e.g. single-stranded or double-stranded). Polynucleotides may be DNA (e.g. genomic DNA or cDNA) or RNA (e.g. mRNA) or combinations thereof. They may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability of the polynucleotide.

The polynucleotide may be delivered in various forms. In some embodiments, a naked polynucleotide may be used, either in linear form, or inserted into a plasmid, such as an expression plasmid. In other embodiments, a live vector such as a viral or bacterial vector may be used.

One or more regulatory sequences that aid in transcription of DNA into RNA and/or translation of RNA into a polypeptide may be present. In some instances, such as in the case of a polynucleotide that is a messenger RNA (mRNA) molecule, regulatory sequences relating to the transcription process (e.g. a promoter) are not required, and protein expression may be effected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject to which the composition of the invention is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

In some embodiments, the vaccine compositions as disclosed herein comprise antigens that are weakly immunogenic. As used herein, by “weakly immunogenic” it is meant that in conventional vaccines (e.g. aqueous vaccines) or when administered by conventional methods, the antigens have little or no ability to induce, maintain and/or boost an immune response.

For example, in an embodiment, a weakly immunogenic antigen is one that when formulated in a non-depot-forming vaccine, such as an aqueous vaccine, is unable to sufficiently prime an immune response. This is in contrast to when the same antigen is formulated in a comparable depot-forming vaccine as disclosed herein (i.e. having the same components, except formulated in a hydrophobic carrier), whereby the antigen is now able to sufficiently prime an immune response. In the preceding context, “sufficiently prime an immune response” means that the antigen is able to induce an immune response to the extent necessary that it can be subsequently maintained and/or boosted by a non-depot-forming vaccine.

In an embodiment, a weakly immunogenic antigen is one that upon first exposure to the subject in a non-depot-forming vaccine, induces no immune response or induces an immune response that is at least 2-fold, 3-fold, 4-fold, 5-fold, 10-fold, 15-fold, 20-fold, 30-fold, 40-fold or 50-fold less efficacious as compared to the immune response induced upon first exposure to the subject in a depot-forming vaccine, as assessed by Enzyme-linked Immunospot assay (ELISPOT). In an embodiment, the immune response induced by the non-depot forming vaccine is at least 10-fold, 15-fold, 20-fold, 30-fold, 40-fold or 50-fold less efficacious than that induced by the depot-forming vaccine, as assessed by ELISPOT.

In an embodiment, a weakly immunogenic antigen is one that when administered in a non-depot-forming vaccine, without prior priming with the same antigen in a depot-forming vaccine, is unable to provide a measurable therapeutic benefit to the subject; whereas a measurable therapeutic benefit can be achieved when the antigen is administered in accordance with the methods described herein. In an embodiment, the measureable therapeutic benefit may, for example, be a reduction in tumor size or an increased cancer survival prognosis. In an embodiment, the measurable therapeutic benefit is a reduction in tumor size of at least 25%, 50%, 75%, 80%, 85%, 90%, 95% or 100%.

Without limitation, weakly immunogenic antigens may include, for example, purified and synthetic antigens (e.g. peptides), self-antigens and/or cancer-associated antigens.

As is well-known in the art, a self-antigen is an antigen that originates from within the body of a subject. The immune system is usually non-reactive against self-antigens under normal homeostatic conditions due, for example, to negative selection of T cells in the thymus. These types of antigens therefore pose a difficulty in the development of targeted immune therapies. Likewise, it has been postulated that weak antigenicity is a root cause of why the immune system typically fails to control tumour growth. Many cancer antigens stimulate a weak, and thus slow, immune response that provides the opportunity and time for tumour cells to develop immune evasion mechanisms and to ultimately gain the upper hand.

For these reasons, among others, weakly immunogenic antigens may represent a particularly suitable type of antigen for use in the methods disclosed herein, which have the ability to potentiate the immune response to an antigen. By priming the immune response to these antigens with a depot-forming vaccine as disclosed herein (e.g. a water-free, oil based vaccine), the subsequent administration of these antigens in a non-depot-forming vaccine (e.g. an aqueous vaccine) may be rendered more effective in maintaining and/or boosting the immune response to that antigen.

In embodiment, the vaccine compositions disclosed herein comprise an antigen that is a self-antigen. In embodiment, the vaccine compositions disclosed herein comprise an antigen that is a cancer-associated antigen. In some embodiments, these antigens are a weakly immunogenic antigen.

The amount of antigen used in a single treatment with a composition as described herein may vary depending on the type of antigen and characteristics of the subject (e.g. size, weight, age, sex, etc). One skilled in the art will be able to determine, without undue experimentation, the effective amount of antigen to use in a particular application. The term “effective amount” as used herein means an amount effective, at dosages and for periods of time necessary, to achieve the desired result.

In an embodiment, the amount of antigen used in a single dose of a composition as described herein may be from 0.001 to 5 mg/unit dose of the composition. In certain embodiments, the amount of antigen will be about 0.250 mg/unit dose of the composition. In certain embodiments, the amount of antigen will be about 1 mg/mL of the composition.

Cancer Associated Antigens

In some embodiments, the antigen may be a cancer or tumor-associated protein or a fragment thereof. Many cancer or tumor-associated proteins are known in the art such as for example, and without limitation, those disclosed in WO 2007/041832.

In some embodiments, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. Thus, in an embodiment, a composition disclosed herein may comprise an antigen associated a virus that is linked to the development of cancer.

In some embodiments, the antigen may be any one that is capable of inducing a specific cytotoxic T-lymphocyte (CTL) immune response that is able to effectively recognize a specific conformation on targeted tumor cells and cause their destruction.

In still further embodiments, the antigen may comprise a peptide sequence selected from the following table:

TABLE 2

| Antigen | Sequence | HLA | Patent |
|---|---|---|---|
| Mart-1/ | AAGIGILTV (SEQ ID NO: 5) | A2 | U.S. Pat. No. 5,844,075 |
| Melan-A | EAAGIGILTV (SEQ ID NO: 6) | A2 | U.S. Pat. No. 5,844,075 |
| | ILTVILGVL (SEQ ID NO: 7) | A2 | U.S. Pat. No. 5,844,075 |
| | AEEAAGIGIL (SEQ ID NO: 8) | B45 | U.S. Pat. No. 7,037,509 |
| | AEEAAGIGILT (SEQ ID NO: 9) | B45 | Unknown |
| MCIR | TILLGIFFL (SEQ ID NO: 10) | A2 | Unknown |
| | FLALIICNA (SEQ ID NO: 11) | A2 | Unknown |
| Gp100 | KTWGQYWQV (SEQ ID NO: 12) | A2 | U.S. Pat. No. 5,844,075 |
| | AMLGTHTMEV (SEQ ID NO: 13) | A2 | Unknown |
| | MLGTHTMEV (SEQ ID NO: 14) | A2 | Unknown |
| | SLADTNSLAV (SEQ ID NO: 15) | A2 | U.S. Pat. No. 5,844,075 |
| | ITDQVPFSV (SEQ ID NO: 16) | A2 | U.S. Pat. No. 5,844,075 |
| | LLDGTATLRL (SEQ ID NO: 17) | A2 | U.S. Pat. No. 5,844,075 |
| | YLEPGPVTA (SEQ ID NO: 18) | A2 | U.S. Pat. No. 5,844,075 |
| | VLYRYGSFSV (SEQ ID NO: 19) | A2 | U.S. Pat. No. 5,844,075 |
| | RLPRIFCSC (SEQ ID NO: 20) | A2 | Unknown |
| | LIYRRRLMK (SEQ ID NO: 21) | A3 | Unknown |
| | ALNFPGSQK (SEQ ID NO: 22) | A3 | Unknown |
| | SLIYRRRLMK (SEQ ID NO: 23) | A3 | Unknown |
| | ALLAVGATK (SEQ ID NO: 24) | A3 | U.S. Pat. No. 6,558,671 |
| | ALLAVGATK (SEQ ID NO: 25) | A3 | U.S. Pat. No. 6,977,074 |
| | VYFFLPDHL (SEQ ID NO: 26) | A24 | Unknown |
| | SNDGPTLI (SEQ ID NO: 27) | Cw8 | Unknown |
| PSA | VSHSFPHPLY (SEQ ID NO: 28) | A1 | U.S. Pat. No. 6,037,135 |
| | FLTPKKLQCV (SEQ ID NO: 29) | A2 | U.S. Pat. No. 6,881,405 |
| | VISNDVCAQV (SEQ ID NO: 30) | A2 | Unknown |
| PSM | HSTNGVTRIY (SEQ ID NO: 31) | A1 | Unknown |
| Tyrosinase | KCDICTDEY (SEQ ID NO: 32) | A1 | U.S. Pat. No. 7,019,112 |
| | SSDYVIPIGTY (SEQ ID NO: 33) | A1 | Unknown |
| | YMDGTMSQV (SEQ ID NO: 34) | A2 | U.S. Pat. No. 6,096,313 |
| | MLLAVLYCL (SEQ ID NO: 35) | A2 | U.S. Pat. No. 6,291,430 |
| | AFLPWHRLF (SEQ ID NO: 36) | A24 | U.S. Pat. No. 6,291,430 |
| | SEIWRDIDF (SEQ ID NO: 37) | B44 | U.S. Pat. No. 6,291,430 |
| | MSLQRQFLR (SEQ ID NO: 38) | A31 | U.S. Pat. No. 5,831,016 |
| TRP1 | SVYDFFVWL (SEQ ID NO: 39) | A2 | U.S. Pat. No. 7,067,120 |
| TRP2 | TLDSQVMSL (SEQ ID NO: 40) | A2 | Unknown |
| | LLGPGRPYR (SEQ ID NO: 41) | A31 | U.S. Pat. No. 5,831,016 |
| p53 | ANDPIFVVL (SEQ ID NO: 42) | Cw8 | Unknown |

In a particular embodiment, the vaccine compositions of the invention may comprise an antigen derived from HPV. In an embodiment, the antigen may be derived from the E6, E7, L1 or L2 protein of HPV.

In an embodiment, the antigen of E6 protein of HPV comprises the peptide sequence TIHDIILECV (T10V; SEQ ID NO: 43). In another embodiment, the antigen of the E7 protein of HPV comprises a peptide sequence of RAHYNIVTF (R9F; SEQ ID NO: 3), YMLDLQPETT (Y10T; SEQ ID NO: 44), YMLDLQPET (Y9T; SEQ ID NO: 45 LLMGTLGIV (L9V; SEQ ID NO: 46), or TLGIVCPI (T8I; SEQ ID NO: 47).

In other embodiment, the antigen derived from HPV may be one or more of the HPV antigens disclosed in WO1993/022338, WO2002/070006, WO2006/115413, WO2008/147187, WO2009/002159 or WO2010/123365.

In another embodiment, the antigen may be derived from a tumor-associated protein, such as for example, a melanoma-associated protein. In a further embodiment, the melanoma-associated protein is a tyrosine related protein-2 (TRP-2) or p53. In one embodiment an antigen derived from a TRP-2 protein comprises the peptide sequence SVYDFFVWL (S9L; SEQ ID NO: 39). In another embodiment, an antigen derived from a TRP-2 protein comprises the peptide sequence VYDFFVWL (V8L; SEQ ID NO: 48). In another embodiment, an antigen derived from a p53 protein comprises a peptide sequence selected from KYMCNSSCM (K9M; wild type p53; SEQ ID NO: 49), KYICNSSCM (mK9M; modified p53; SEQ ID NO: 50), and AKXVAAWTLKAAAKYICNSSCM (mK9M; SEQ ID NO: 51), wherein X may be cyclohexylalanyl.

In an embodiment, the antigen contained in the vaccine compositions may comprise a mixture of one or more of the antigens described herein, optionally fused together as a fused protein with or without spacer sequences between the antigens.

In other embodiments, and without limitation, the antigen may be from a membrane surface-bound cancer-associated protein. The surface-bound cancer-associated protein (or antigen thereof) may be capable of being recognized by an antibody.

In a particular embodiment, the vaccine compositions of the invention may comprise one or more survivin antigens.

Survivin, also called baculoviral inhibitor of apoptosis repeat-containing 5 (BIRC5), is a protein involved in the negative regulation of apoptosis. It has been classed as a member of the family of inhibitors of apoptosis proteins (IAPs). Survivin is a 16.5 kDa cytoplasmic protein containing a single BIR motif and a highly charged carboxy-terminal coiled region instead of a RING finger. The gene coding for survivin is nearly identical to the sequence of Effector Cell Protease Receptor-1 (EPR-1), but oriented in the opposite direction. The coding sequence for the survivin (homo sapiens) is 429 nucleotides long including stop codons:

| SEQ ID NO: 52 | |
|---|---|
| atgggtgccc cgacgttgcc ccctgcctgg cagccctttc tcaaggacca ccgcatctct | 60 |
| acattcaaga actggccctt cttggagggc tgcgcctgca ccccggagcg gatggccgag | 120 |
| gctggcttca tccactgccc cactgagaac gagccagact tggcccagtg tttcttctgc | 180 |
| ttcaaggagc tggaaggctg ggagccagat gacgacccca tagaggaaca taaaaagcat | 240 |
| tcgtccggtt gcgctttcct ttctgtcaag aagcagtttg aagaattaac ccttggtgaa | 300 |
| tttttgaaac tggacagaga aagagccaag aacaaaattg caaaggaaac caacaataag | 360 |
| aagaaagaat ttgaggaaac tgcgaagaaa gtgcgccgtg ccatcgagca gctggctgcc | 420 |
| atggattga | 429 |

The encoded protein survivin (homo sapiens) is 142 amino acids long:

```
SEQ ID NO: 53
Met Gly Ala Pro Thr Leu Pro Pro Ala Trp Gln Pro Phe Leu Lys Asp
1 5 10 15
His Arg Ile Ser Thr Phe Lys Asn Trp Pro Phe Leu Glu Gly Cys Ala
                        20 25 30
Cys Thr Pro Glu Arg Met Ala Glu Ala Gly Phe Ile His Cys Pro Thr
                35 40 45
Glu Asn Glu Pro Asp Leu Ala Gln Cys Phe Phe Cys Phe Lys Glu Leu
        50 55 60
Glu Gly Trp Glu Pro Asp Asp Asp Pro Ile Glu Glu His Lys Lys His
65 70 75 80
Ser Ser Gly Cys Ala Phe Leu Ser Val Lys Lys Gln Phe Glu Glu Leu
                                85 90 95
Thr Leu Gly Glu Phe Leu Lys Leu Asp Arg Glu Arg Ala Lys Asn Lys
                        100 105 110
Ile Ala Lys Glu Thr Asn Asn Lys Lys Lys Glu Phe Glu Glu Thr Ala
                115 120 125
Lys Lys Val Arg Arg Ala Ile Glu Gln Leu Ala Ala Met Asp
        130 135 140
```

It is postulated that the survivin protein functions to inhibit caspase activation, thereby leading to negative regulation of apoptosis or programmed cell death. Consistent with this function, survivin has been identified as one of the top genes invariably up-regulated in many types of cancer but not in normal tissue (see e.g. Altieri et al., Lab Invest, 79: 1327-1333, 1999; and U.S. Pat. No. 6,245,523). This fact therefore makes survivin an ideal target for cancer therapy as cancer cells are targeted while normal cells are not. Indeed, survivin is highly expressed in many tumor types, including a large portion of human cancer, and has reported prognostic value.

In some embodiments, vaccines of the invention may comprise one or more survivin antigens. As used herein, the term “survivin antigen” encompasses any peptide, polypeptide or variant thereof (e.g. survivin peptide variant) derived from a survivin protein or a fragment thereof. The term “survivin antigen” also encompasses a polynucleotide that encodes a survivin peptide, survivin peptide variant or survivin peptide functional equivalent described herein. Polynucleotides may be DNA (e.g. genomic DNA or cDNA) or RNA (e.g. mRNA) or combinations thereof. They may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability of the polynucleotide.

In an embodiment, the survivin antigen may comprise the full length survivin polypeptide or a nucleic acid encoding the full length survivin polypeptide. Alternatively, the survivin antigen may be a survivin peptide comprising a fragment of any length of the survivin protein. Exemplary embodiments include a survivin peptide that comprises at least 5, 6, 7, 8, 9, 10, 11, 12, 13, 14, 15, 16, 17, 18, 19 or 20 amino acid residues. In specific embodiments, the survivin peptide consists of a heptapeptide, an octapeptide, a nonapeptide, a decapeptide or an undecapeptide, consisting of 7, 8, 9, 10, 11 consecutive amino acid residues of the survivin protein (e.g. SEQ ID NO: 53), respectively. Particular embodiments of the survivin antigen include survivin peptides of about 9 or 10 amino acids.

Survivin antigens of the invention also encompass variants and functional equivalents of survivin peptides. Variants or functional equivalents of a survivin peptide encompass peptides that exhibit amino acid sequences with differences as compared to the specific sequence of the survivin protein, such as one or more amino acid substitutions, deletions or additions, or any combination thereof. The difference may be measured as a reduction in identity as between the survivin protein sequence and the survivin peptide variant or survivin peptide functional equivalent.

The identity between amino acid sequences may be calculated using algorithms well known in the art. Survivin peptide variants or functional equivalents are to be considered as falling within the meaning of a “survivin antigen” of the invention when they are, over their entire length, at least 70% identical to a peptide sequence of a survivin protein, such as at least 75% identical, at least 80% identical, at least 85% identical, at least 90% identical, or at least 95% identical, including 96%, 97%, 98% or 99% identical with a peptide sequence of a survivin protein. In a particular embodiment, the survivin peptide variant has a sequence that is at least 85%, 90%, 95%, 96%, 97%, 98% or 99% identical to a consecutive amino acid sequence of SEQ ID NO: 53.

The survivin protein from which the survivin antigen can be derived is a survivin protein from any animal species in which the protein is expressed. A particular embodiment is the survivin protein from humans (SEQ ID NO: 53). Based on the sequence of the selected survivin protein, the survivin antigen may be derived by any appropriate chemical or enzymatic treatment of the survivin protein or coding nucleic acid. Alternatively, the survivin antigen may be synthesized by any conventional peptide or nucleic acid synthesis procedure with which the person of ordinary skill in the art is familiar.

The survivin antigen (peptide or nucleic acid) may have a sequence which is a native sequence of survivin. Alternatively, the survivin antigen may be a peptide or nucleic acid sequence modified by one or more substitutions, deletions or additions, such as e.g. the survivin peptide variants or functional equivalents described herein. Exemplary procedures and modifications of survivin peptides that increase the immunogenicity of the peptides include, for example, those described in WO 2004/067023 involving amino acid substitutions introduced at anchor positions which increase peptide binding to the HLA class I molecule.

In an embodiment, the survivin antigen is any peptide derived from the survivin protein, or any survivin peptide variant thereof, that is capable of binding MHC Class I HLA molecules. Along these lines, the survivin antigen may be any survivin peptide, or survivin peptide variant thereof, that is capable of inducing or potentiating an immune response in a subject.

In an embodiment, the survivin antigen is a peptide antigen comprising an amino acid sequence from the survivin protein (e.g. SEQ ID NO: 53) that is capable of eliciting a cytotoxic T-lymphocyte (CTL) response in a subject, or a nucleic acid molecule encoding said peptide.

In an embodiment, the vaccine comprises one or more synthetic survivin peptides, or variants thereof, based on the amino acid sequence of the survivin protein, such as the amino acid sequence set forth in SEQ ID NO: 53.

Survivin peptides, survivin peptide variants and survivin functional equivalents, and their use for diagnostic and therapeutic purposes, specifically in cancer, have been described, for example, in WO 2004/067023 and WO 2006/081826. The novel peptides disclosed in these publications were found to be capable of eliciting cytotoxic T-lymphocyte (CTL) responses in cancer patients. In particular, in WO 2004/067023, it was found that MHC Class I restricted peptides can be derived from the survivin protein, which are capable of binding to MEW Class I HLA molecules and thereby eliciting both ex vivo and in situ CTL immune responses in patients suffering from a wide range of cancer diseases.

In an embodiment, a vaccine composition of the invention may include any one or more of the survivin peptides, survivin peptide variants or survivin peptide functional equivalents disclosed in WO 2004/067023 and WO 2006/081826.

In another embodiment, a vaccine composition of the invention may include one or more of a survivin peptide, survivin peptide variant or survivin peptide functional equivalent having the ability to bind any of the MHC Class I molecules selected from HLA-A, HLA-B or HLA-C molecules.

Exemplary MEW Class I HLA-A molecules to which the survivin peptide, survivin peptide variant, or survivin peptide functional equivalent may bind include, without limitation, HLA-A1, HLA-A2, HLA-A3, HLA-A9, HLA-A10, HLA-A11, HLA-A19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, and HLA-A69.

Exemplary MEW Class I HLA-B molecules to which the survivin peptide, survivin peptide variant, or survivin peptide functional equivalent may bind include, without limitation, HLA-B5, HLA-B7, HLA-B8, HLA-B12, HLA-B13, HLA-B14, HLA-B15, HLA-B16, HLA-B17, HLA-B18, HLA-B21, HLA-B22, HLA-B27, HLA-B35, HLA-B37, HLA-B38, HLA-B39, HLA-B40, HLA-B41, HLA-B42, HLA-B44, HLA-B45, HLA-B46 and HLA-B47.

Exemplary MEW Class I HLA-C molecules to which the survivin peptide, survivin peptide variant, or survivin peptide functional equivalent may bind include, without limitation, HLA-C1, HLA-C2, HLA-C3, HLA-C4, HLA-C5, HLA-C6, HLA-C7 and HLA-C16.

In a particular embodiment, a vaccine composition of the invention may comprise one or more of the survivin peptide antigens selected from:

| | | |
|---|---|---|
| i) | FEELTLGEF (SEQ ID NO: 54) | [HLA-A1] |
| ii) | FTELTLGEF (SEQ ID NO: 55) | [HLA-A1] |
| iii) | LTLGEFLKL (SEQ ID NO: 56) | [HLA-A2] |
| iv) | LMLGEFLKL (SEQ ID NO: 2) | [HLA-A2] |
| v) | RISTFKNWPF (SEQ ID NO: 57) | [HLA-A3] |
| vi) | RISTFKNWPK (SEQ ID NO:58) | [HLA-A3] |
| vii) | STFKNWPFL (SEQ ID NO: 59) | [HLA-A24] |
| viii) | LPPAWQPFL (SEQ ID NO: 60) | [HLA-B7] |

The above-listed survivin peptides represent, without limitation, exemplary MHC Class I restricted peptides encompassed by the invention. The specific MHC Class I HLA molecule to which each of the survivin peptides is believed to bind is shown on the right in square brackets. A vaccine of the invention may comprise one or more of these survivin peptides, in any suitable combination.

In a further embodiment, a vaccine composition of the invention may comprise any one or more of the five survivin peptides listed below, in any suitable combination:

| | | |
|---|---|---|
| i) | FTELTLGEF (SEQ ID NO: 55) | [HLA-A1] |
| ii) | LMLGEFLKL (SEQ ID NO: 2) | [HLA-A2] |
| iii) | RISTFKNWPK (SEQ ID NO: 58) | [HLA-A3] |
| iv) | STFKNWPFL (SEQ ID NO: 59) | [HLA-A24] |
| v) | LPPAWQPFL (SEQ ID NO: 60) | [HLA-B7] |

In a particular embodiment, the composition of the invention comprises all five of the survivin peptide antigens listed above.

In some embodiments, in addition to the at least one survivin antigen, a vaccine composition of the invention may comprise one or more additional antigens, such as for example those described herein.

CTL Epitopes and B Cell Epitopes

As mentioned above, in some embodiments, the antigen is a molecule comprising at least one B cell epitope or CTL epitope.

The epitopes may be of any chemical nature, including without limitation peptides, carbohydrates, lipids, glycopeptides and glycolipids. In particular embodiments, the epitopes are peptides derived from any of the antigens described herein. The epitope may be identical to a naturally occurring epitope, or may be a modified form of a naturally occurring epitope.

B cell epitopes are epitopes recognized by B cells and by antibodies. B cell peptide epitopes are typically at least five amino acids, more often at least six amino acids, still more often at least seven or eight amino acids in length, and may be continuous (“linear”) or discontinuous (“conformational”); the latter being formed, for example, by the folding of a protein to bring non-contiguous parts of the primary amino acid sequence into physical proximity. B cell epitopes may also be carbohydrate epitopes.

In an embodiment, the antigen of the compositions described herein may be or comprise a B cell epitope capable of inducing a humoral immune response.

In some embodiments, the antigen of the compositions described herein may be or comprise a B cell epitope associated with an infectious disease. For example, the antigen may be or comprise a B cell epitope derived from a virus, such as for example influenza virus or respiratory syncytial virus. In another embodiment, the B cell epitope may be an epitope derived from the hemagglutinin glycoprotein of the H5N1 influenza virus.

In another embodiment, the antigen of the compositions described herein may be or comprise a B cell epitope derived from a bacterium, such as for example Bordetella pertussis or Bacillus anthracis. In a particular embodiment, the B cell epitope may be an epitope of the pertussis toxoid protein produced by Bordetella pertussis. In another particular embodiment, the B cell epitope may be an epitope of the anthrax recombinant protective antigen (rPA) or the anthrax mutant recombinant protective antigen (mrPA).

In another embodiment, the antigen of the compositions described herein may be or comprise a B cell epitope derived from a protozoan, such as from the genus Plasmodium.

In a further embodiment, the composition may comprise a mixture of B cell epitopes as antigens for inducing a humoral immune response. The B cell epitopes may be linked to form a single polypeptide.

CTL epitopes are molecules recognized by cytotoxic T lymphocytes. CTL epitopes are typically presented on the surface of an antigen-presenting cell, complexed with MHC molecules. As used herein, the term “CTL epitope” refers to a molecule (e.g. peptide) which is substantially the same as a natural CTL epitope of an antigen (including a hapten). The CTL epitope may be modified as compared to its natural counterpart, such as by one or two amino acids. Unless otherwise stated, reference herein to a CTL epitope is to an unbound molecule that is capable of being taken up by cells and presented on the surface of an antigen-presenting cell.

The CTL epitope should typically be one that is amendable to recognization by T cell receptors so that a cell-mediated immune response can occur. For peptides, CTL epitopes may interact with class I or class II MHC molecules. CTL epitopes presented by MHC class I molecules are typically peptides between 8 and 15 amino acids in length, and more often between 9 and 11 amino acids in length. CTL epitopes presented by MHC class II molecules are typically peptides between 5 and 24 amino acids in length, and more often between 13 and 17 amino acids in length. If the antigen is larger than these sizes, it will be processed by the immune system into fragments of a size more suitable for interaction with MHC class I or II molecules. Therefore, CTL epitopes may be part of larger peptide than those mentioned above.

Many CTL epitopes are known. Several techniques of identifying additional CTL epitopes are recognized by the art. In general, these involve preparing a molecule which potentially provides a CTL epitope and characterizing the immune response to that molecule.

In an embodiment, the antigen of the compositions described herein may be or comprise a CTL epitope capable of inducing a CTL response. For example, the antigen may be a CTL epitope derived from a virus, such as HPV.

In another embodiment, the antigen may be or comprise a CTL epitope derived from the E6 or E7 protein of HPV. For example, and without limitation, the CTL epitope of E6 protein of HPV may comprise the peptide sequence TIHDIILECV (T10V; SEQ ID NO: 43) and the CTL epitope of the E7 protein of HPV may comprise the peptide sequence RAHYNIVTF (R9F; SEQ ID NO: 3), YMLDLQPETT (Y10T; SEQ ID NO: 44), YMLDLQPET (Y9T; SEQ ID NO: 45) LLMGTLGIV (L9V; SEQ ID NO: 46), and TLGIVCPI (T81; SEQ ID NO: 47).

In another embodiment, the CTL epitope may be an epitope of a tumor-associated protein, such as for example, one or more of the survivin peptides described herein or a melanoma-associated protein. In an embodiment, the melanoma-associated protein may be a tyrosine related protein-2 (TRP-2) or p53, which can be obtained by various methods including recombinant technology or chemical synthesis.

For example, and without limitation, the CTL epitope of a TRP-2 derived protein may comprise the peptide sequence SVYDFFVWL (S9L; SEQ ID NO: 39) or VYDFFVWL (V8L; SEQ ID NO: 48). The CTL epitope of a p53 derived protein may comprise, for example, the peptide sequence KYMCNSSCM (K9M; wild type p53; SEQ ID NO: 49), KYICNSSCM (mK9M; modified p53; SEQ ID NO: 50) or AKXVAAWTLKAAAKYICNSSCM (mK9M; SEQ ID NO: 51) wherein X may be cyclohexylalanyl.

In a further embodiment, the composition may comprise a mixture of CTL epitopes as antigens for inducing a CTL response. The CTL epitopes may be linked to form a single polypeptide.

In some embodiments, the B cell and CTL epitopes are disease-associated and/or disease-specific epitopes. Such diseases include, but are not limited to, any of those described earlier herein. For example, and without limitation, the disease may be a cancer (such as, for example, breast cancer, ovarian cancer, prostate cancer, glioblastoma or diffuse large B cell lymphoma), an infectious disease (such as, for example, a disease caused by or associated with human papillomavirus (HPV) infection, respiratory syncytial virus (RSV) infection, influenza virus infection, Ebola virus infection, Bacillus anthracis infection, or Plasmodium malariae infection) or an addiction disease (such as, for example, addiction to cocaine).

T-Helper Epitopes

In some embodiments, the vaccine compositions of the invention, including both the depot-forming and non-depot-forming vaccines, may also comprise at least one T-helper epitope or T-helper antigen.

T-helper epitopes are a sequence of amino acids (natural or non-natural amino acids) that have T-helper activity. T-helper epitopes are recognised by T-helper lymphocytes, which play an important role in establishing and maximising the capabilities of the immune system, and are involved in activating and directing other immune cells, such as for example cytotoxic T lymphocytes.

A T-helper epitope can consist of a continuous or discontinuous epitope. Hence not every amino acid of a T-helper is necessarily part of the epitope. Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis et al. (1988) J. Immunol. 140:1808-1815; Demotz et al. (1989) J. Immunol. 142:394-402; Chong et al. (1992) Infect. Immun. 60:4640-4647). The T-helper domain of the subject peptides may have from about 10 to about 50 amino acids, and more particularly about 10 to about 30 amino acids. When multiple T-helper epitopes are present, then each T-helper epitope acts independently.

In some embodiments, the T-helper epitope may form part of an antigen described herein. In particular, if the antigen is of sufficient size, it may contain an epitope that functions as a T-helper epitope. In other embodiments, the T-helper epitope is a separate molecule from the antigen.

In another embodiment, T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

In a particular embodiment, the compositions of the invention may comprise as a T-helper epitope or antigen, the modified Tetanus toxin peptide A16L (830 to 844; AQYIKANSKFIGITEL (SEQ ID NO: 61), with an alanine residue added to its amino terminus to enhance stability (Slingluff et al., Clin Cancer Res., 7: 3012-3024, 2001).

Other sources of T-helper epitopes which may be used in the present compositions include, for example, hepatitis B surface antigen helper T cell epitopes, pertussis toxin helper T cell epitopes, measles virus F protein helper T cell epitope, Chlamydia trachomitis major outer membrane protein helper T cell epitope, diphtheria toxin helper T cell epitopes, Plasmodium falciparum circumsporozoite helper T cell epitopes, Schistosoma mansoni triose phosphate isomerase helper T cell epitopes, Escherichia coli TraT helper T cell epitopes and immune-enhancing analogs and segments of any of these T-helper epitopes.

In some embodiments, the T-helper epitope may be a universal T-helper epitope. A universal T-helper epitope as used herein refers to a peptide or other immunogenic molecule, or a fragment thereof, that binds to a multiplicity of MHC class II molecules in a manner that activates T cell function in a class II (CD4+ T cells)-restricted manner. An example of a universal T-helper epitope is PADRE (pan-DR epitope) comprising the peptide sequence AKXVAAWTLKAAA (SEQ ID NO: 62), wherein X may be cyclohexylalanyl. PADRE specifically has a CD4+ T-helper epitope, that is, it stimulates induction of a PADRE-specific CD4+ T-helper response.

In addition to the modified tetanus toxin peptide A16L mentioned earlier, Tetanus toxoid has other T-helper epitopes that work in the similar manner as PADRE. Tetanus and diphtheria toxins have universal epitopes for human CD4+ cells (Diethelm-Okita, B. M. et al., J. Infect. Diseases, 181:1001-1009, 2000). In another embodiment, the T-helper epitope may be a tetanus toxoid peptide such as F21E comprising the peptide sequence FNNFTVSEWLRVPKVSASHLE (amino acids 947-967) (SEQ ID NO: 63).

In certain embodiments, the T-helper epitope is fused to at least one of the one or more antigens in the vaccine of the invention (e.g. a fusion peptide).

The amount of T-helper epitope used in a single treatment with a composition as described herein may vary depending on the type of T-helper epitope and characteristics of the subject (e.g. size, weight, age, sex, etc). One skilled in the art will be able to determine, without undue experimentation, the appropriate amount of T-helper epitope to use in a particular application.

In an embodiment, the amount of T-helper epitope used in a single dose of a composition as described herein may be from 0.001 to 5 mg/unit dose of the composition. In certain embodiments, the amount of antigen will be about 0.125 mg/unit dose of the composition. In certain embodiments, the amount of antigen will be about 500 μg/mL of the composition.

Adjuvants

In some embodiments, the vaccine compositions disclosed herein, including both the depot-forming and non-depot-forming vaccines, may comprise one or more adjuvants.

A large number of adjuvants have been described and are known to those skilled in the art. See, for example, Remington's Pharmaceutical Sciences (Remington's Pharmaceutical Sciences, Mack Publishing Company, Easton, Pa., USA 1985) and The United States Pharmacopoeia: The National Formulary (USP 24 NF19) published in 1999.

Exemplary adjuvants include, without limitation, alum, other compounds of aluminum, Bacillus of Calmette and Guerin (BCG), TiterMax™, Ribi™, Freund's Complete Adjuvant (FCA), CpG-containing oligodeoxynucleotides (CpG ODN), lipopeptides and polyl:C polynucleotides. An exemplary CpG ODN is 5′-TCCATGACGTTCCTGACGTT-3′ (SEQ ID NO: 64). The skilled person can readily select other appropriate CpG ODNs on the basis of the target species and efficacy. An exemplary lipopeptide includes, without limitation, Pam3Cys-SKKK (EMC Microcollections, Germany) or variants, homologs and analogs thereof. The Pam2 family of lipopeptides has been shown to be an effective alternative to the Pam3 family of lipopeptides.

In some embodiments, the pharmaceutical or vaccine compositions may comprise a polyl:C polynucleotide as an adjuvant, such as for example and without limitation, a 26 mer deoxy inosine/cytosine synthetic polynucleotide.

As used herein, a “polyl:C” or “polyl:C polynucleotide” is a double-stranded polynucleotide molecule (RNA or DNA or a combination of DNA and RNA), each strand of which contains at least 6 contiguous inosinic or cytidylic acid residues, or at least 6 contiguous residues selected from inosinic acid and cytidylic acid in any order (e.g. IICIIC, ICICIC or IIICCC), and which is capable of inducing or enhancing the production of at least one inflammatory cytokine, such as interferon, in a mammalian subject. Polyl:C polynucleotides will typically have a length of about 8, 10, 12, 14, 16, 18, 20, 22, 24, 25, 28, 30, 35, 40, 45, 50, 55, 60, 65, 70, 75, 80, 85, 90, 95, 100, 150, 200, 250, 300, 500, 1000 or more residues. The upper limit is not believed to be essential. Polyl:C polynucleotides will often have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides.

Each strand of a polyl:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g. uridine), provided there is at least one contiguous region of 6 I, 6 C or 6 I/C residues as described above. Typically, each strand of a polyl:C polynucleotide will contain no more than 1 non-1/C residue per 6 I/C residues, more particularly no more than 1 non-1/C residue per every 8, 10, 12, 14, 16, 18, 20, 22, 24, 26, 28 or 30 I/C residues.

The inosinic acid or cytidylic acid (or other) residues in the polyl:C polynucleotide may be derivatized or modified as is known in the art, provided the ability of the polyl:C polynucleotide to promote the production of an inflammatory cytokine, such as interferon, is retained. Non-limiting examples of derivatives or modifications include e.g. azido modifications, fluoro modifications, or the use of thioester (or similar) linkages instead of natural phosphodiester linkages to enhance stability in vivo. The polyl:C polynucleotide may also be modified to e.g. enhance its resistance to degradation in vivo by e.g. complexing the molecule with positively charged poly-lysine and carboxymethylcellulose, or with a positively charged synthetic peptide.

If present, the polyl:C polynucleotide will typically be included in the compositions in an amount from about 0.001 mg to 1 mg/unit dose of the composition. In certain embodiments, the amount of polyl:C polynucleotide will be about 0.1 mg/unit dose of the composition. In certain embodiments, the amount of polyl:C polynucleotide will be about 400 μg/mL of the composition.

Other suitable adjuvants of the compositions disclosed herein are those that activate or increase the activity of TLR2. As used herein, an adjuvant which “activates” or “increases the activity” of a TLR2 includes any adjuvant, in some embodiments a lipid-based adjuvant, which acts as a TLR2 agonist. Further, activating or increasing the activity of TLR2 encompasses its activation in any monomeric, homodimeric or heterodimeric form, and particularly includes the activation of TLR2 as a heterodimer with TLR1 or TLR6 (i.e. TLR1/2 or TLR2/6). Exemplary embodiments of an adjuvant that activates or increases the activity of TLR2 include lipid-based adjuvants, such as those described in WO2013/049941.

In another embodiment, a composition of the invention may comprise a lipid A mimic or analog adjuvant, such as for example those disclosed in International Patent Application No. PCT/CA2015/051309 and the references cited therein. In a particular embodiment, the adjuvant may be JL-265 or JL-266 as disclosed in PCT/CA2015/051309.

Further examples of adjuvants that may be used include, without limitation, chemokines, colony stimulating factors, cytokines, 1018 ISS, aluminum salts, Amplivax, AS04, AS15, ABM2, Adjumer, Algammulin, AS01B, AS02 (SBASA), ASO2A, BCG, Calcitriol, Chitosan, Cholera toxin, CP-870,893, CpG, polyI:C, CyaA, DETOX (Ribi Immunochemicals), Dimethyldioctadecylammonium bromide (DDA), Dibutyl phthalate (DBP), dSLIM, Gamma inulin, GM-CSF, GMDP, Glycerol, IC30, IC31, Imiquimod, ImuFact IMP321, IS Patch, ISCOM, ISCOMATRIX, JuvImmune, LipoVac, LPS, lipid core protein, MF59, monophosphoryl lipid A and analogs or mimics thereof, Montanide® IMS1312, Montanide® based adjuvants (e.g. Montanide ISA-51, -50 and -70), OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, Saponin, synthetic polynucleotides, Zymosan, pertussis toxin.

Accordingly, the compositions herein may comprise one or more pharmaceutically acceptable adjuvants. In some embodiments, at least one of the antigens may be coupled to at least one of the adjuvants.

The amount of adjuvant used depends on the amount of antigen and on the type of adjuvant. One skilled in the art can readily determine the amount of adjuvant needed in a particular application by empirical testing.

Hydrophobic Carrier

The depot-forming vaccine compositions of the invention comprise a hydrophobic carrier, such as for example a liquid hydrophobic substance.

The hydrophobic carrier may be an essentially pure hydrophobic substance or a mixture of hydrophobic substances. Although it is possible that the hydrophobic carrier may be an emulsion of water in a hydrophobic substance or an emulsion of water in a mixture of hydrophobic substances (i.e. water-in-oil emulsion), the exclusion of water may represent a preferred embodiment for formulating a depot-forming vaccine as disclosed herein.

Thus, in particular embodiments, the depot-forming vaccines disclosed herein may be water-free. By “water-free” it is meant that the depot-forming vaccine contains no water at all. In another embodiment, the depot-forming vaccines disclosed herein may be substantially free of water. The term “substantially free of water” is intended to encompass embodiments where the hydrophobic carrier may still contain small quantities of water, provided that the water is present in the non-continuous phase of the carrier. For example, individual components of the composition may have bound water that may not be completely removed by processes such as lyophilization or evaporation and certain hydrophobic carriers may contain small amounts of water dissolved therein. Generally, compositions of the invention that are “substantially free of water” contain, for example, less than about 10%, 9%, 8%, 7%, 6%, 5%, 4%, 3%, 2%, 1%, 0.5%, 0.1%, 0.05% or 0.01% water on a weight/weight basis of the total weight of the carrier component of the composition.

Hydrophobic substances that are useful in the compositions described herein are those that are pharmaceutically and/or immunologically acceptable. The carrier is typically a liquid but certain hydrophobic substances that are not liquids at atmospheric temperature may be liquefied, for example by warming, and may also be useful.

Oil or a mixture of oils is a particularly suitable carrier for use in the depot-forming vaccine compositions disclosed herein. Oils should be pharmaceutically and/or immunologically acceptable. Suitable oils include, for example, mineral oils (especially light or low viscosity mineral oil such as Drakeol® 6VR), vegetable oils (e.g., soybean oil), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier.

In another embodiment, the hydrophobic carrier may be, or comprise, a mannide oleate in mineral oil solution, such as that commercially available as Montanide® ISA 51 (SEPPIC, France).

To enhance immunogenicity of vaccines, Immunovaccine Inc. has developed an adjuvanting vaccine platform designed to facilitate a strong and robust immune response to peptide or polynucleotide antigens. DepoVax™ (DPX) is a lipid-in-oil formulation that can be formulated with any antigen, or mixture of antigens, to induce or potentiate a cell-mediated immune response (Karkada et al., J Immunother 33(3):250-261, 2010) and/or a humoral immune response. DPX forms a strong depot at the site of immunization which prolongs antigen exposure to the immune system.

It has been shown that a single vaccination with peptide or polynucleotide antigens in DPX results in equivalent or better immune responses than multiple vaccinations with the same antigens in other conventional formulations, such as Montanide ISA51 VG emulsions, similar to VacciMax which was a first generation emulsion-based vaccine platform (Daftarian et al., J Transl Med 5: 26, 2007; Mansour et al., J Transl Med 5: 20, 2007). A DepoVax™ based peptide-vaccine called DPX-0907 has completed a phase I clinical trial in breast, ovarian and prostate cancer patients demonstrating safety and immunogenicity in these advanced patients (Berinstein et al., J Transl Med 10(1): 156, 2012).

Unlike water-in-oil emulsion based vaccines, which rely on oil entrapping water droplets containing antigen and adjuvant, DepoVax™ based formulations rely on lipids to facilitate the incorporation of antigens and adjuvants directly into the oil, without the need for emulsification. Advantages of this approach include: (1) enhancing the solubility of hydrophilic antigens/adjuvant in oil diluents which otherwise would normally have maximum solubility in aqueous based diluents, and (2) the elimination of cumbersome emulsification procedures prior to vaccine administration.

In some embodiments, the hydrophobic carrier of the depot-forming vaccine compositions disclosed herein may be Immunovaccine, Inc's adjuvanting system DepoVax™

Emulsifiers

In some embodiments, the vaccine compositions disclosed herein may comprise one or more emulsifiers. The emulsifier may be a pure emulsifying agent or a mixture of emulsifying agents. The emulsifier(s) should be pharmaceutically and/or immunologically acceptable.

The use of an emulsifier may be of particular relevance to the depot-forming vaccines disclosed herein. For instance, in some embodiments an emulsifier may be used to assist in stabilizing the amphiphile, mixture of amphiphile and antigen, or the mixture of amphiphile, antigen and other vaccine components (e.g. adjuvant, T-helper epitope, etc.) when the amphiphile or mixtures are resuspended into the hydrophobic carrier. The use of an emulsifier may, for example, promote more even distribution of the amphiphile or mixture in the hydrophobic carrier.

The emulsifier may be amphipathic and therefore, the emulsifier may include a broad range of compounds. In some embodiments, the emulsifier may be a surfactant, such as for example, a non-ionic surfactant. Examples of emulsifiers which may be used include polysorbates, which are oily liquids derived from polyethylene glycolyated sorbital, and sorbitan esters. Polysorbates may include, for example, sorbitan monooleate. Typical emulsifiers are well-known in the art and include, without limitation, mannide oleate (Arlacel™ A), lecithin, Tween™ 80, Spans™ 20, 80, 83 and 85. In an embodiment, the emulsifier for use in the vaccine compositions is mannide oleate.

The emulsifier is generally pre-mixed with the hydrophobic carrier. In some embodiments, a hydrophobic carrier which already contains an emulsifier may be used. For example, a hydrophobic carrier such Montanide™ ISA-51 already contains the emulsifier mannide oleate. In other embodiments, the hydrophobic carrier may be mixed with emulsifier before combining with the amphiphile, mixture of amphiphile and antigen, or the mixture of amphiphile, antigen and other vaccine components (e.g. adjuvant, T-helper epitope, etc.).

The emulsifier is used in an amount effective to promote even distribution of the amphiphile in the hydrophobic carrier and/or to assist in the formation of structures, assemblies or arrays described herein. Typically, the volume ratio (v/v) of hydrophobic carrier to emulsifier is in the range of about 5:1 to about 15:1, more particularly 10:1.

In an embodiment, the depot-forming vaccine comprises or consists of: (i) five survivin peptide antigens comprising the amino acid sequences FTELTLGEF (SEQ ID NO: 55), LMLGEFLKL (SEQ ID NO: 2), RISTFKNWPK (SEQ ID NO:58), STFKNWPFL (SEQ ID NO:59), and LPPAWQPFL (SEQ ID NO: 60); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) the hydrophobic carrier Montanide® ISA 51 VG.

In an embodiment, the depot-forming and non-depot-forming vaccine (DPX-Survivac) comprise or consist of the following: five human leukocyte antigen (HLA)-restricted epitopes (HLA-A1: FTELTLGEF (SEQ ID NO: 55), HLA-A2: LMLGEFLKL (SEQ ID NO: 2), HLA-A3: RISTFKNWPK (SEQ ID NO:58), HLA-A24: STFKNWPFL (SEQ ID NO:59), and HLA-B7: LPPAWQPFL (SEQ ID NO: 60)), a universal T-helper epitope from tetanus toxoid (AQYIKANSKFIGITEL; SEQ ID NO: 61), a poly I:C polynucleotide adjuvant, and liposomes consisting of DOPC and cholesterol. These components are formulated in a phosphate buffer, filled into vials and lyophilized to a dry cake. For the depot-forming vaccine, the cake is re-suspended in lx volume of the hydrophobic carrier Montanide ISA51 VG (SEPPIC, France). For the non-depot-forming vaccine, the dry cake is re-suspended using 2× volume of sterile water instead of a hydrophobic carrier. Exemplary dose volumes and deliverables in the final vaccine formulations are shown in Table 3.

TABLE 3
Components of each dose of an oil-based DPX-Survivac
vaccine and an aqueous-based DPX-Survivac vaccine.

| | | |
|---|---|---|
| Component | DPX-Survivac (Oil) | DPX-Survivac (Aqueous) |

| | | |
|---|---|---|
| Diluent | Montanide ISA51 VG | Sterile water |

| | | | | |
|---|---|---|---|---|
| Dose volume | 0.250 | milliliters | 0.500 | milliliters |
| Antigens | 0.250 | milligrams | 0.250 | milligrams |
| T-helper | 0.125 | milligrams | 0.125 | milligrams |
| Adjuvant | 0.1 | milligrams | 0.1 | milligrams |
| DOPC | 30 | milligrams | 30 | milligrams |
| cholesterol | 3 | milligrams | 3 | milligrams |

In another embodiment, the depot-forming vaccine comprises or consists of: (i) a peptide antigen derived from human papillomavirus (HPV); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) the hydrophobic carrier Montanide® ISA 51 VG.

In an embodiment, the non-depot-forming vaccine comprises: (i) five survivin peptide antigens comprising the amino acid sequences FTELTLGEF (SEQ ID NO: 55), LMLGEFLKL (SEQ ID NO: 2), RISTFKNWPK (SEQ ID NO:58), STFKNWPFL (SEQ ID NO:59), and LPPAWQPFL (SEQ ID NO: 60); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) a water carrier.

In another embodiment, the non-depot-forming vaccine comprises: (i) a peptide antigen derived from human papillomavirus (HPV); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) a water carrier.

Methods for Preparing the Vaccine Compositions

The depot-forming and non-depot-forming vaccine compositions may be prepared by known methods in the art having regard to the present disclosure, including the non-limiting methods described in the examples. For preparing the depot-forming vaccines, specific reference may be made, for example, to WO1996/014871 and WO2009/043165 as exemplary disclosures of the preparation of vaccine and immunogenic compositions comprising antigens, amphiphiles and a hydrophobic carrier. Methods for preparing non-depot-forming vaccines (e.g. aqueous-based vaccines) are well known in the art, and any of these methods may be employed.

Exemplary embodiments for preparing the vaccine compositions disclosed herein are described below, without limitation.

As used in this section, the term “antigen” is used generally to describe how an antigen may be formulated in the vaccine compositions of the invention. The term “antigen” encompasses both the singular form “antigen” and the plural “antigens”. It is not necessary that all antigens be introduced into the vaccine composition in the same way.

In an embodiment for preparing the vaccines, the antigen and optionally other vaccine components (e.g. T-helper epitope and adjuvant) are reconstituted in a suitable solvent together with an amphiphile. The vaccine components are then dried to form a dry cake, and the dry cake is resuspended in a hydrophobic carrier to prepare the depot-forming vaccine or resuspended in an aqueous carrier to prepare the non-depot-forming vaccine. The step of drying may be performed by various means known in the art, such as by freeze-drying, lyophilization, rotary evaporation, evaporation under pressure, etc. Low heat drying that does not compromise the integrity of the components can also be used. Heat can also be used to assist in resuspending the antigen/amphiphile mixture.

The “suitable solvent” is one that is suitable for solubilizing the antigen and/or amphiphile, and can be determined by the skilled person. In an embodiment, a polar protic solvent such as an alcohol (e.g tert-butanol, n-butanol, isopropanol, n-propanol, ethanol or methanol), water, acetate buffer, formic acid or chloroform can be used. In some cases, the same solvent can be used to solubilize both the amphiphile and the antigen, and the solubilized antigen and solubilized amphiphile are then mixed. Alternatively, the antigen and amphiphile may be mixed prior to solubilization, and then solubilized together. In a further alternative, only one of the amphiphile or the antigen is solubilized, and the non-solubilized component added.

In a particular embodiment, to prepare the vaccines the antigen and adjuvant are reconstituted in acetate buffer (0.1 M, pH 9.5) with DOPC and cholesterol (Lipoid, Germany). These vaccine components are then lyophilized to form a dry cake. Just prior to injection, the dry cake is resuspended in ISA51 VG oil (SEPPIC, France) to prepare an oil-based depot-forming vaccine or resuspended in water to prepare an aqueous-based non-depot-forming vaccine.

In another embodiment, to prepare the vaccines the antigen is reconstituted in 30% tert-butanol with DOPC and cholesterol (Lipoid, Germany). To this antigen-lipid mixture, adjuvant is added and lyophilized to form a dry cake. Just prior to injection, the dry cake is resuspended in ISA51 VG oil (SEPPIC, France) to prepare an oil-based depot-forming vaccine or resuspended in water to prepare an aqueous-based non-depot-forming vaccine.

In the above embodiments, removal (drying) of the solvent leaves the vaccine components, including the antigen, in an array of amphiphile molecules with their hydrophilic head groups oriented towards the vaccine components. The vaccine components and amphiphile can then be suspended in the hydrophobic carrier, such as an oil, since they have been made sufficiently hydrophobic as described herein.

In another embodiment, to prepare the vaccines the antigen(s) and optionally T-helper epitope and/or adjuvant, are formulated in phosphate buffer with DOPC and cholesterol (Lipoid, Germany). These vaccine components are then lyophilized to form a dry cake. Just prior to injection, the dry cake is resuspended in Montanide ISA51 VG oil (SEPPIC, France) to prepare an oil-based depot-forming vaccine or resuspended in water to prepare an aqueous-based non-depot-forming vaccine.

Additional components as described herein, such as T-helper epitope and adjuvant, may be added at any stage in the formulation process. For instance, one or more such additional components may be combined with the antigen or amphiphile either before or after solubilization, or added to the solubilized mixture. In another embodiment, the additional components may instead be added to or combined with the dried mixture of antigen and amphiphile, or combined with the hydrophobic or aqueous carrier either before or after resuspension of the dry mixture of antigen and amphiphile in the hydrophobic carrier. In an embodiment, the T-helper epitope is added to the vaccine composition in the same way as the antigen. In an embodiment, the antigen and T-helper epitope are a fused peptide.

In some embodiments, it may be appropriate to include an emulsifier in the hydrophobic carrier to assist in stabilizing the vaccine components of the dry cake when they are resuspended in the hydrophobic carrier. The emulsifier is provided in an amount sufficient to resuspend the dry mixture of antigen and amphiphile in the hydrophobic carrier and maintain the antigen and amphiphile in suspension in the hydrophobic carrier. For example, the emulsifier may be present at about 5% to about 15% weight/weight or weight/volume of the hydrophobic carrier.

As will be appreciated, it is typically not necessary that the non-depot-forming vaccine include an amphiphile. Therefore, in some embodiments, the non-depot-forming vaccine may be prepared without an amphiphile.

Stabilizers such as sugars, anti-oxidants, or preservatives that maintain the biological activity or improve chemical stability to prolong the shelf life of any of the vaccine components, may be added to such compositions.

Immune Responses and Treatment Indications

The present disclosure relates to methods for potentiating an immune response to an antigen in a subject, corresponding uses, and vaccines and kits which may be used in such methods.

As referred to herein, the “immune response” may either be a cell-mediated immune response or a humoral immune response.

In a particular embodiment, the methods disclosed herein may be used for potentiating a cytotoxic T-lymphocyte (CTL) immune response.

As used herein, the terms “cell-mediated immune response”, “cellular immunity”, or “cytotoxic T-lymphocyte (CTL) immune response” (used interchangeably herein) refer to an immune response characterized by the activation of macrophages and natural killer cells, the production of antigen-specific cytotoxic T lymphocytes and/or the release of various cytokines in response to an antigen. Cytotoxic T lymphocytes are a sub-group of T lymphocytes (a type of white blood cell) which are capable of inducing the death of infected somatic or tumor cells; they kill cells that are infected with viruses (or other pathogens), or that are otherwise damaged or dysfunctional.

Most cytotoxic T cells express T cell receptors that can recognise a specific peptide antigen bound to Class I MHC molecules. Typically, cytotoxic T cells also express CD8 (i.e. CD8+ T cells), which is attracted to portions of the Class I MHC molecule. This affinity keeps the cytotoxic T cell and the target cell bound closely together during antigen-specific activation.

Cellular immunity protects the body by, for example, activating antigen-specific cytotoxic T-lymphocytes (e.g. antigen-specific CD8+ T cells) that are able to lyse body cells displaying epitopes of foreign antigen on their surface, such as virus-infected cells, cells with intracellular bacteria, and cancer cells displaying tumor antigens; activating macrophages and natural killer cells, enabling them to destroy intracellular pathogens; and stimulating cells to secrete a variety of cytokines that influence the function of other cells involved in adaptive immune responses and innate immune responses.

Cellular immunity is an important component of the adaptive immune response and following recognition of antigen by cells through their interaction with antigen-presenting cells such as dendritic cells, B lymphocytes and to a lesser extent, macrophages, protects the body by various mechanisms such as:

1. activating antigen-specific cytotoxic T-lymphocytes that are able to induce apoptosis in body cells displaying epitopes of foreign antigen on their surface, such as virus-infected cells, cells with intracellular bacteria, and cancer cells displaying tumor antigens;

2. activating macrophages and natural killer cells, enabling them to destroy intracellular pathogens; and

3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Since cell-mediated immunity involves the participation of various cell types and is mediated by different mechanisms, several methods could be used to demonstrate the induction of immunity following vaccination. These could be broadly classified into detection of: i) specific antigen presenting cells; ii) specific effector cells and their functions and iii) release of soluble mediators such as cytokines.

i) Antigen presenting cells: Dendritic cells and B cells (and to a lesser extent macrophages) are equipped with special immunostimulatory receptors that allow for enhanced activation of T cells, and are termed professional antigen presenting cells (APC). These immunostimulatory molecules (also called co-stimulatory molecules) are up-regulated on these cells following infection or vaccination, during the process of antigen presentation to effector cells such as CD4 and CD8 cytotoxic T cells. Such co-stimulatory molecules (such as CD40, CD80, CD86, MHC class I or MHC class II) can be detected, for example, by using flow cytometry with fluorochrome-conjugated antibodies directed against these molecules along with antibodies that specifically identify APC (such as CD11c for dendritic cells).

ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens. These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perforin and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cell-mediated immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flowcytometric measurement of intracellular IFN-yin these cells.

CD4+ “helper” T cells: CD4+ lymphocytes, or helper T cells, are immune response mediators, and play an important role in establishing and maximizing the capabilities of the adaptive immune response. These cells have no cytotoxic or phagocytic activity; and cannot kill infected cells or clear pathogens, but, in essence “manage” the immune response, by directing other cells to perform these tasks. Two types of effector CD4+ T helper cell responses can be induced by a professional APC, designated Th1 and Th2, each designed to eliminate different types of pathogens.

Helper T cells express T cell receptors (TCR) that recognize antigen bound to Class Il MHC molecules. The activation of a naive helper T cell causes it to release cytokines, which influences the activity of many cell types, including the APC that activated it. Helper T cells require a much milder activation stimulus than cytotoxic T cells. Helper T cells can provide extra signals that “help” activate cytotoxic cells. Two types of effector CD4+ T helper cell responses can be induced by a professional APC, designated Th1 and Th2, each designed to eliminate different types of pathogens. The two Th cell populations differ in the pattern of the effector proteins (cytokines) produced. In general, Th1 cells assist the cell-mediated immune response by activation of macrophages and cytotoxic T cells; whereas Th2 cells promote the humoral immune response by stimulation of B cells for conversion into plasma cells and by formation of antibodies. For example, a response regulated by Th1 cells may induce lgG2a and lgG2b in mouse (IgGI and lgG3 in humans) and favor a cell mediated immune response to an antigen. If the IgG response to an antigen is regulated by Th2 type cells, it may predominantly enhance the production of IgGI in mouse (IgG2 in humans). The measure of cytokines associated with Th1 or Th2 responses will give a measure of successful vaccination. This can be achieved by specific ELISA designed for Th1-cytokines such as IFN-γ, IL-2, IL-12, TNF-α and others, or Th2-cytokines such as IL-4, IL-5, IL-10 among others.

iii) Measurement of cytokines: released from regional lymph nodes gives a good indication of successful immunization. As a result of antigen presentation and maturation of APC and immune effector cells such as CD4 and CD8 T cells, several cytokines are released by lymph node cells. By culturing these LNC in vitro in the presence of antigen, antigen-specific immune response can be detected by measuring release if certain important cytokines such as IFN-γ, IL-2, IL-12, TNF-α and GM-CSF. This could be done by ELISA using culture supernatants and recombinant cytokines as standards.

Successful immunization may be determined in a number of ways known to the skilled person including, but not limited to, hemagglutination inhibition (HAIJ) and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g. in the identification of activated or memory lymphocytes. A skilled person may also determine if immunization with a composition of the invention elicited an antibody and/or cell mediated immune response using other known methods. See, for example, Current Protocols in Immunology Coligan et al., ed. (Wiley Interscience, 2007).

In an embodiment, the methods disclosed herein may be used for potentiating an antibody immune response.

An “antibody immune response” or “humoral immune response” (used interchangeably herein), as opposed to cell-mediated immunity, is mediated by secreted antibodies which are produced in the cells of the B lymphocyte lineage (B cells). Such secreted antibodies bind to antigens, such as for example those on the surfaces of foreign substances, pathogens (e.g. viruses, bacteria, etc.) and/or cancer cells, and flag them for destruction.

As used herein, “humoral immune response” refers to antibody production and may also include, in addition or alternatively, the accessory processes that accompany it, such as for example the generation and/or activation of T-helper 2 (Th2) or T-helper 17 (Th17) cells, cytokine production, isotype switching, affinity maturation and memory cell activation. “Humoral immune response” may also include the effector functions of an antibody, such as for example toxin neutralization, classical complement activation, and promotion of phagocytosis and pathogen elimination. The humoral immune response is often aided by CD4+ Th2 cells and therefore the activation or generation of this cell type may also be indicative of a humoral immune response. The term “humoral immune response” is used interchangeably herein with “antibody response” or “antibody immune response”.

An “antibody” is a protein comprising one or more polypeptides substantially or partially encoded by immunoglobulin genes or fragments of immunoglobulin genes. The recognized immunoglobulin genes include the κ, λ, α, γ, δ, ε and μ constant region genes, as well as myriad immunoglobulin variable region genes. Light chains are classified as either κ or λ. Heavy chains are classified as γ, μ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigen to T cells, such as T helper CD4 or cytotoxic CD8+ T cells, thus propagating the immune response. B cells, as well as T cells, are part of the adaptive immune response. During an active immune response, induced for example by either vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the TLRs.

Antigen presenting cells, such as dendritic cells and B cells, are drawn to vaccination sites and can interact with antigens and adjuvants contained in a vaccine composition. Typically, the adjuvant stimulates the cells to become activated and the antigen provides the blueprint for the target. Different types of adjuvants may provide different stimulation signals to cells. For example, poly I:C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle et al., Curr Med Chem, 2008; So., J lmmunol, 2012).

A humoral immune response is one of the common mechanisms for effective infectious disease vaccines (e.g. to protect against viral or bacterial invaders). However, a humoral immune response can also be useful for combating cancer. Whereas a cancer vaccine is typically designed to produce a cell-mediated immune response that can recognize and destroy cancer cells, B cell mediated responses may target cancer cells through other mechanisms which may in some instances cooperate with a cytotoxic T cell for maximum benefit. Examples of B cell mediated (e.g. humoral immune response mediated) anti-tumor responses include, without limitation: 1) Antibodies produced by B cells that bind to surface antigens found on tumor cells or other cells that influence tumorigenesis. Such antibodies can, for example. induce killing of target cells through antibody-dependant cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with a tumor or tumor-associated cells to modulate a signaling or cellular pathway that supports cancer; and 4) Antibodies that bind to intracellular targets and mediate anti-tumor activity through a currently unknown mechanism.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Without limitation, other assays that may be used to detect the presence of an antigen-specific antibody include immunological assays (e.g. radioimmunoassay (RIA)), immunoprecipitation assays, and protein blot (e.g. Western blot) assays; and neutralization assays (e.g., neutralization of viral infectivity in an in vitro or in vivo assay).

The methods and vaccine compositions described herein may be useful for treating or preventing diseases and/or disorders ameliorated by a cell-mediated immune response or a humoral immune response. The methods and vaccines find application in any instance in which it is desired to administer an antigen to a subject to induce a cell-mediated immune response or a humoral immune response.

“Treating” or “treatment of”, or “preventing” or “prevention of”, as used herein, refers to an approach for obtaining beneficial or desired results, including clinical results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc.

In an embodiment, the methods and compositions disclosed herein may be used for treating and/or preventing cancer in a subject in need thereof. The subject may have cancer or may be at risk of developing cancer.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

Without limitation, cancers that may be capable of being treated and/or prevented by the use or administration of a composition of the invention include carcinoma, adenocarcinoma, lymphoma, leukemia, sarcoma, blastoma, myeloma, and germ cell tumors. Without limitation, particularly suitable embodiments may include glioblastoma, multiple myeloma, ovarian cancer, breast cancer, fallopian tube cancer, prostate cancer or peritoneal cancer. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. In another embodiment, the cancer may be one that expresses one or more cancer-specific antigens (e.g. survivin).

In a particular embodiment, the cancer is breast cancer, ovarian cancer, prostate cancer, fallopian tube cancer, peritoneal cancer, glioblastoma or diffuse large B cell lymphoma.

The methods and compositions disclosed herein may be useful for either the treatment or prophylaxis of cancer; for example, a reduction of the severity of cancer (e.g. size of the tumor, aggressiveness and/or invasiveness, malignancy, etc) or the prevention of cancer recurrences.

In another embodiment, the methods and compositions disclosed herein may be used for treating and/or preventing an infectious disease, such as caused by a viral infection, in a subject in need thereof. The subject may be infected with a virus or may be at risk of developing a viral infection. Viral infections that may be treated and/or prevented by the use or administration of a composition of the invention include, without limitation, Cowpoxvirus, Vaccinia virus, Pseudocowpox virus, Human herpesvirus 1, Human herpesvirus 2, Cytomegalovirus, Human adenovirus A-F, Polyomavirus, Human papillomavirus (HPV), Parvovirus, Hepatitis A virus, Hepatitis B virus, Hepatitis C virus, Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella. In a particular embodiment, the viral infection is Human papillomavirus, Ebola virus, Human respiratory syncytial virus or an influenza virus.

In another embodiment, the methods or compositions disclosed herein may be used for treating and/or preventing an infectious disease, such as caused by a non-viral pathogen (such as a bacterium or protozoan) in a subject in need thereof. The subject may be infected with the pathogen or may be at risk of developing an infection by the pathogen. Without limitation, exemplary bacterial pathogens may include Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica. In a particular embodiment, the bacterial infection is Anthrax. Without limitation, exemplary protozoan pathogens may include those of the genus Plasmodium (Plasmodium falciparum, Plasmodium malariae, Plasmodium vivax, Plasmodium ovale or Plasmodium knowlesi), which cause malaria.

In another embodiment, the methods or compositions disclosed herein may be used for treating and/or preventing a neurodegenerative disease in a subject in need thereof, wherein the neurodegenerative disease is associated with the expression of an antigen. The subject may have a neurodegenerative disease or may be at risk of developing a neurodegenerative disease. Neurodegenerative diseases that may be treated and/or prevented by the methods or compositions disclosed herein include, without limitation, Alzheimer's disease, Parkinson's disease, Huntington's disease, and amyotrophic lateral sclerosis (ALS).

In another embodiment, the methods or compositions disclosed herein may be used for treating and/or preventing an addiction disease (such as, for example, addiction to cocaine).

Kits, Combinations and Reagents

For practicing the methods of the present invention, the compositions as described herein may optionally be provided to a user as a kit. For example, a kit of the invention contains one or more components of the vaccine compositions of the invention. The kit can further comprise one or more additional reagents, packaging material, containers for holding the components of the kit, and an instruction set or user manual detailing preferred methods of using the kit components. In an embodiment, the containers are vials.

In an embodiment, the kit contains both of the depot-forming and non-depot-forming vaccines pre-formulated in separate containers in a ready-to-use format. As an example, in an embodiment, the kit comprises at least one container comprising a depot-forming vaccine, said depot-forming vaccine comprising one or more antigens and a hydrophobic carrier; and at least one container comprising a non-depot-forming vaccine, said non-depot-forming vaccine comprising the one or more antigens and an aqueous carrier.

In an alternative embodiment, the kit contains only one of either the depot-forming vaccine or non-depot-forming vaccine pre-formulated in a container in ready-to-use format. The other vaccine may be provided with all components, except carrier, in one container (e.g. dry cake) ready for reconstitution in the appropriate carrier (e.g. hydrophobic carrier or aqueous carrier) or as individual components in separate containers for formulation and reconstitution in the appropriate carrier (e.g. hydrophobic carrier or aqueous carrier). In a particular embodiment, it is the non-depot-forming vaccine that is provided in pre-formulated ready-to-use format, whereas the components of the depot-forming vaccine are provided in a form that requires reconstitution or formulation and reconstitution in the hydrophobic carrier.

In various aspects of the above kit embodiments, in addition to antigen and carrier, the depot-forming vaccine and/or non-depot-forming vaccine may optionally further comprise one or more of a T-helper epitope, an adjuvant, an amphiphile and an emulsifier. These components may be provided individually in separate containers or may be provided as any combination thereof together in a container.

In a further alternative embodiment, the kit contains at least one container comprising the vaccine components, except the appropriate carrier. In this embodiment, the kit can comprise in additional separate containers one or both of the hydrophobic carrier and the aqueous carrier for reconstituting the vaccine components. In these embodiments, the vaccine components may be in the form of a dry cake that is ready to be re-suspended in the appropriate carrier.

In an embodiment, the kit comprises one container comprising one or more antigens and optionally one or more of a T-helper epitope, an adjuvant, an amphiphile and an emulsifier; at least one container comprising a hydrophobic carrier; and at least one container comprising an aqueous carrier, wherein the container comprising the one or more antigens and optionally one or more of a T-helper epitope, an adjuvant, an amphiphile comprises a sufficient quantity of each component to prepare both the depot-forming vaccine and the non-depot-forming vaccine by reconstitution with the hydrophobic carrier or the aqueous carrier.

In another embodiment, the kit comprises at least two containers, each container comprising one or more antigens and optionally one or more of a T-helper epitope, an adjuvant, an amphiphile and an emulsifier; at least one container comprising a hydrophobic carrier; and at least one container comprising an aqueous carrier, wherein at least one container of antigen and optionally T-helper epitope, adjuvant, amphiphile and emulsifier is for reconstitution with the hydrophobic carrier to prepare a depot-forming vaccine and at least one container of antigen and optionally T-helper epitope, adjuvant, amphiphile and emulsifier is for reconstitution with the aqueous carrier to prepare a non-depot-forming vaccine.

In a particular embodiment, the kit comprises at least two containers, each container comprising one or more antigens, a T-helper epitope, an adjuvant and lipids; at least one container comprising a hydrophobic carrier; and at least one container comprising an aqueous carrier, wherein at least one container of antigen, T-helper epitope, adjuvant and lipids is for reconstitution with the hydrophobic carrier to prepare a depot-forming vaccine and at least one container of antigen, T-helper epitope, adjuvant and lipids is for reconstitution with the aqueous carrier to prepare a non-depot-forming vaccine.

In other embodiments, the depot-forming and non-depot-forming vaccines may be provided in separate kits. In an embodiment, in each respective kit the depot-forming or non-depot-forming vaccine are provided as single ready-to-use vials. In another embodiment, in each respective kit any number of the components of the depot-forming or non-depot-forming vaccine are provided individually, or as any combination of components, in separate containers to be formulated and reconstituted in the appropriate carrier.

In an embodiment of the kit, the T-helper epitope is a peptide comprising the amino acid sequence FNNFTVSFWLRVPKVSASHLE (SEQ ID NO: 63).

In an embodiment of the kit, the adjuvant is a polyl:C polynucleotide.

In an embodiment of the kit, the amphiphile is one or more lipids, such as phospholipids. In an embodiment, the lipids are 1,2-dioleoyl-sn-glycero-3-phosphocholine (DOPC) and cholesterol.

In an embodiment of the kit, the one or more antigens comprise one or more survivin antigens as described herein. In a particular embodiment of the kit, the one or more antigens comprise a mixture of five peptide antigens comprising the amino acid sequence FTELTLGEF (SEQ ID NO: 55); LMLGEFLKL (SEQ ID NO: 2); RISTFKNWPK (SEQ ID NO: 58); STFKNWPFL (SEQ ID NO: 59) or LPPAWQPFL (SEQ ID NO: 60).

In an embodiment of the kit, the antigen is a peptide antigen derived from human papillomavirus (HPV).

In an embodiment, the kit may additionally contain an agent that interferes with DNA replication. The agent that interferes with DNA replication may be included in the kit with a separate container, or the agent may be included with other components. In a particular embodiment, the agent that interferes with DNA replication that is included in the kit is an alkylating agent, such as for example, cyclophosphamide.

The skilled person will appreciate that alternate arrangements of the kit are possible and are encompassed by the disclosure herein.

The kit as disclosed herein may be used in practicing the methods disclosed herein. In an embodiment, the kit is for use in potentiating an immune response in a subject by priming the immune response with the depot-forming vaccine and maintaining and/or boosting the immune response with the non-depot-forming vaccine. In an embodiment that may be particularly suitable, the depot-forming vaccine is water-free or substantially free of water.

The present disclosure also relates to a combination for practicing the methods disclosed herein. In an embodiment, the invention relates to the combination of a depot-forming vaccine comprising one or more antigens in a hydrophobic carrier and a non-depot-forming vaccine comprising the same one or more antigens, for use in a method as disclosed herein. The depot-forming and non-depot-forming vaccines may be any one of the embodiments disclosed herein.

More particularly, in an embodiment the present disclosure relates to the combination of a depot-forming vaccine comprising one or more antigens in a hydrophobic carrier and a non-depot-forming vaccine comprising the one or more antigens, for use in a method for potentiating an immune response to an antigen in a subject, wherein the subject is administered at least one dose of the depot-forming vaccine; and the subject is subsequently administered at least one dose of the non-depot-forming vaccine. As above, the depot-forming and non-depot-forming vaccines may be any one of the embodiments disclosed herein.

### Embodiments

Particular embodiments of the present disclosure include, without limitation, the following:

(1) A method for potentiating an immune response to an antigen in a subject, said method comprising: (i) administering to the subject at least one dose of a depot-forming vaccine comprising one or more antigens in a hydrophobic carrier; and (ii) subsequently administering to the subject at least one dose of a non-depot-forming vaccine comprising the one or more antigens.

(2) The method according to paragraph (1), wherein each of the at least one dose of the depot-forming vaccine is a priming dose that is capable of inducing an immune response to the one or more antigens.

(3) The method according to paragraph (1) or (2), wherein each of the at least one dose of the non-depot-forming vaccine is a maintenance or boosting dose that is capable of maintaining and/or boosting the immune response to the one or more antigens.

(4) The method according to paragraph (3), which comprises administering a first maintenance or boosting dose of the non-depot-forming vaccine within about 1 day, 1 week, 2 weeks, 3 weeks, 4 weeks, 5 weeks, 6 weeks, 7 weeks, 8 weeks, 9 weeks or 10 weeks of a final priming dose of the depot-forming vaccine.

(5) The method according to paragraph (3), which comprises administering a first maintenance or boosting dose of the non-depot-forming vaccine within about 1 day, 1 week, 2 weeks, 3 weeks, or 4 weeks of a final priming dose of the depot-forming vaccine.

(6) The method according to any one of paragraphs (1) to (5), wherein the at least one dose of the depot-forming vaccine is one, two, three, four or five doses.

(7) The method according to any one of paragraphs (1) to (6), wherein the at least one dose of the depot-forming vaccine is one or two doses.

(8) The method according to any one of paragraphs (1) to (7), wherein the at least one dose of the non-depot-forming vaccine is one, two, three, four or five doses.

(9) The method according to any one of paragraphs (1) to (7), wherein the at least one dose of the non-depot-forming vaccine is a continuous repeated dosing once every day, once every week, once every two weeks, once every three weeks, or once monthly.

(10) The method according to any one of paragraphs (1) to (9), wherein after a first dose of the non-depot-forming vaccine, each subsequent dose of the non-depot-forming vaccine is administered within about 1 day, 1 week, 2 weeks, 3 weeks, or 4 weeks of the immediately preceding dose.

(11) The method according to any one of paragraphs (1) to (10), wherein after a first dose of the depot-forming vaccine, each subsequent dose of the depot-forming vaccine is administered within about 1 day, 1 week, 2 weeks, 3 weeks, or 4 weeks of the immediately preceding dose.

(12) The method according to any one of paragraphs (1) to (11), which comprises administering two doses of the depot-forming vaccine prior to administration of the non-depot-forming vaccine.

(13) The method according to any one of paragraphs (1) to (10), which comprises administering one dose of the depot-forming vaccine prior to administration of the non-depot-forming vaccine.

(14) The method according to paragraph (12), which comprises administering the depot-forming vaccine on day 0 and day 21.

(15) The method according to paragraph (12), which comprises administering the depot-forming vaccine on day 0 and day 21, and administering the non-depot-forming vaccine on day 42.

(16) The method according to paragraph (13), which comprises administering the depot-forming vaccine on day 0, and administering the non-depot-forming vaccine on day 21 and day 42.

(17) The method according to any one of paragraphs (14) to (16), wherein administering the non-depot-forming vaccine continues once every three weeks after day 42.

(18) The method according to any one of paragraphs (1) to (17), which further comprises administering to the subject an agent that interferes with DNA replication.

(19) The method according to paragraph (18), wherein the agent that interferes with DNA replication is cyclophosphamide.

(20) The method according to paragraph (19), which comprises a cycle of low dose metronomic cyclophosphamide.

(21) The method according to paragraphs (20), wherein the cycle comprises administering the cyclophosphamide to the subject daily for a period of 7 consecutive days, beginning every second week.

(22) The method according to paragraph (21), wherein the cyclophosphamide is first administered 7 days prior to the first administration with the depot-forming vaccine.

(23) The method according to any one of paragraphs (1) to (22), which further comprises administering to the subject an immune response checkpoint inhibitor.

(24) The method according to paragraph (23), wherein the immune response checkpoint inhibitor is an inhibitor of Programmed Death-Ligand 1 (PD-L1), Programmed Death 1 (PD-1), CTLA-4, PD-L2, LAG3, TIM3, 41BB, 2B4, A2aR, B7H1, B7H3, B7H4, BTLA, CD2, CD27, CD28, CD30, CD40, CD70, CD80, CD86, CD160, CD226, CD276, DR3, GAL9, GITR, HVEM, IDO1, IDO2, inducible T cell costimulatory (ICOS), KIR, LAIR1, LIGHT, macrophage receptor with collageneous structure (MARCO), phosphatidylserine (PS), OX-40, SLAM, TIGIT, VISTA, VTCN1, or any combination thereof.

(25) The method according to any one of paragraphs (1) to (24), wherein the hydrophobic carrier of the depot-forming vaccine is an oil or a mixture of oils.

(26) The method according to paragraph (25), wherein the hydrophobic carrier comprises a vegetable oil, nut oil, or mineral oil.

(27) The method according to paragraph (25), wherein the hydrophobic carrier is mineral oil or is a mannide oleate in mineral oil solution, for example Montanide® ISA 51.

(28) The method according to any one of paragraphs (1) to (27), wherein the depot-forming vaccine is substantially free of water.

(29) The method according to any one of paragraphs (1) to (27), wherein the depot-forming vaccine is water-free.

(30) The method according to any one of paragraphs (25) to (29), wherein the one or more antigens are sufficiently hydrophobic, or are made sufficiently hydrophobic, such that the one or more antigens are miscible in the oil.

(31) The method according to paragraph (30), wherein the one or more antigens are naturally hydrophobic.

(32) The method according to paragraph (30), wherein the hydrophobicity of the one or more antigens is increased by modification of the antigen.

(33) The method according to paragraph (32), wherein the one or more antigens are peptide antigens modified by lipidation.

(34) The method according to paragraph (33), wherein the lipidation is one or more of N-terminal myristoylation; C-terminal attachment of cholesterol; S-prenylation of a cysteine residue at or close to the C-terminus; S-palmitoylation of a cysteine residue; and attachment of a lipid having an adjuvanting activity.

(35) The method according to paragraph (34), wherein the lipid having an adjuvanting activity comprises dipalmitoyl-S-glyceryl-cysteine (PAM₂Cys), tripalmitoyl-S-glyceryl-cysteine (PAM₃Cys), palmitic acid, or other lipoamino acids.

(36) The method according to paragraph (35), wherein the lipid having an adjuvanting activity is Pam-2-Cys-Ser-(Lys)4 or Pam-3-Cys-Ser-(Lys)4.

(37) The method according to paragraph (30), wherein the one or more antigens are non-covalently complexed to a hydrophobic molecule, compound or complex using hydrophobic ion-pairing.

(38) The method according to paragraph (37), wherein the hydrophobic ion-pairing technique comprises forming an immunogenic complex by electrostatic interaction between a positively charged antigen and a negatively charged organic molecule, compound or complex.

(39) The method according to paragraph (38), wherein the negatively charged organic molecule, compound or complex is a saponin or a saponin complex.

(40) The method according to paragraph (30), wherein the one or more antigens are made sufficiently hydrophobic by the presence of an amphiphile in the depot-forming vaccine.

(41) The method according to paragraph (40), wherein the amphiphile is closely associated with the one or more antigens to make the one or more antigens miscible in the hydrophobic carrier.

(42) The method according to paragraph (41), wherein the amphiphile forms a sheet or vesicular structure, partially or completely surrounding the one or more antigens.

(43) The method according to any one of paragraphs (40) to (42), wherein the amphiphile is a lipid.

(44) The method according to paragraph (43), wherein the lipids form a closed vesicular structure around the one or more antigens.

(45) The method according to paragraph (44), wherein the closed vesicular structure is a single layer vesicular structure (e.g. a micelle) or a bilayer vesicular structure (e.g. a unilamellar or multilamellar liposome).

(46) The method according to any one of paragraphs (43) to (45), wherein the lipid is a phospholipid.

(47) The method according to any one of paragraphs (1) to (46), wherein the non-depot-forming vaccine comprises an aqueous carrier.

(48) The method according to paragraph (47), wherein the aqueous carrier is water or phosphate buffered saline (PBS).

(49) The method according to any one of paragraphs (1) to (48), wherein the depot-forming vaccine and/or the non-depot-forming vaccine further comprise an adjuvant.

(50) The method according to paragraph (49), wherein the adjuvant is a polyI:C polynucleotide.

(51) The method according to any one of paragraphs (1) to (50), wherein the one or more antigens are: (i) derived from a virus, bacterium or protozoan, such as for example Ebola virus, human papillomavirus (HPV), influenza virus, respiratory syncytial virus, Bordetella pertussis, Bacillus anthracis or Plasmodium malariae; (ii) a membrane surface-bound cancer antigen, such as for example a survivin antigen; or (iii) a toxin, such as for example cocaine.

(52) The method according to any one of paragraphs (1) to (51), wherein the one or more antigens comprise at least one B cell epitope, at least one CTL epitope or a combination thereof.

(53) The method according to any one of paragraphs (1) to (52), wherein the antigen is a survivin antigen.

(54) The method according to paragraph (53), wherein the survivin antigen is a peptide antigen comprising an amino acid sequence from the survivin protein (SEQ ID NO: 53), or a nucleic acid molecule encoding said peptide antigen.

(55) The method according to any one of paragraphs (1) to (52), wherein the antigen is a peptide antigen comprising the amino acid sequence FEELTLGEF (SEQ ID NO: 54); FTELTLGEF (SEQ ID NO: 55); LTLGEFLKL (SEQ ID NO: 56); LMLGEFLKL (SEQ ID NO: 2); RISTFKNWPF (SEQ ID NO: 57); RISTFKNWPK (SEQ ID NO:58); STFKNWPFL (SEQ ID NO: 59); and LPPAWQPFL (SEQ ID NO: 60), or any combination thereof; or a nucleic acid molecule encoding said peptide antigen.

(56) The method according to any one of paragraphs (1) to (52), wherein the one or more antigens comprise a mixture of five peptide antigens comprising the amino acid sequence FTELTLGEF (SEQ ID NO: 55); LMLGEFLKL (SEQ ID NO: 2); RISTFKNWPK (SEQ ID NO:58); STFKNWPFL (SEQ ID NO: 59) or LPPAWQPFL (SEQ ID NO: 60).

(57) The method according to any one of paragraphs (1) to (52), wherein the antigen is a peptide antigen derived from human papillomavirus (HPV) or a nucleic acid molecule encoding said peptide antigen.

(58) The method according to paragraph (57), wherein the peptide antigen derived from HPV comprises the amino acid sequence YMLDLQPETT (SEQ ID NO: 44), YMLDLQPET (SEQ ID NO: 45); LLMGTLGIV (SEQ ID NO: 46) or TLGIVCPI (SEQ ID NO: 47).

(59) The method according to any one of paragraphs (1) to (52), wherein the antigen is a self-antigen.

(60) The method according to any one of paragraphs (1) to (52), wherein the antigen is a cancer-associated antigen.

(61) The method according to any one of paragraphs (1) to (60), wherein the antigen is a weakly immunogenic antigen.

(62) The method according to any one of paragraphs (1) to (61), wherein the depot-forming vaccine and/or the non-depot-forming vaccine further comprise a T-helper epitope.

(63) The method according to paragraph (62), wherein the T-helper epitope is a peptide comprising the amino acid sequence FNNFTVSFWLRVPKVSASHLE (SEQ ID NO: 63).

(64) The method according to any one of paragraphs (1) to (24), wherein the depot-forming vaccine comprises: (i) five survivin peptide antigens comprising the amino acid sequences FTELTLGEF (SEQ ID NO: 55), LMLGEFLKL (SEQ ID NO: 2), RISTFKNWPK (SEQ ID NO: 58), STFKNWPFL (SEQ ID NO: 59), and LPPAWQPFL (SEQ ID NO: 60); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) the hydrophobic carrier Montanide® ISA 51 VG.

(65) The method according to paragraph (64), wherein the non-depot-forming vaccine comprises the same components (i), (ii), (iii) and (iv), and a water carrier.

(66) The method according to any one of paragraphs (1) to (24), wherein the depot-forming vaccine comprises: (i) a peptide antigen derived from human papillomavirus (HPV); (ii) a universal T-helper epitope from tetanus toxoid comprising the amino acid sequence AQYIKANSKFIGITEL (SEQ ID NO: 61); (iii) a polyl:C polynucleotide adjuvant; (iv) a lipid molecule mixture of 1,2-dioleoyl-sn-glycero-3-phosphocholine (DOPC) and cholesterol lipid mixture; and (v) the hydrophobic carrier Montanide® ISA 51 VG.

(67) The method according to paragraph (66), wherein the non-depot-forming vaccine comprises the same components (i), (ii), (iii) and (iv), and a water carrier.

(68) The method according to any one of paragraphs (1) to (67), which is for potentiating a cytotoxic T-lymphocyte (CTL) immune response in the subject.

(69) The method according to any one of paragraphs (1) to (52), which is for potentiating an antibody immune response in the subject.

(70) The method according to any one of paragraphs (1) to (69), wherein the subject has had no prior immune response towards the antigen.

(71) The method according to any one of paragraphs (68) to (70), which is for treating or preventing cancer; an infectious disease; or an addiction disease.

(72) The method according to paragraph (68), which is for treating or preventing cancer.

(73) The method according to paragraph (71) or (72), wherein the cancer is breast cancer, ovarian cancer, prostate cancer, fallopian tube cancer, peritoneal cancer, glioblastoma or diffuse large B cell lymphoma.

(74) The method according to any one of paragraphs (1) to (73), wherein the non-depot-forming vaccine is cleared from an injection site faster than the depot-forming vaccine.

(75) The method according to paragraph (74), wherein potentiation of the immune response comprises a reduction in the occurrence of injection site reactions.

(76) Use of a depot-forming vaccine in combination with a non-depot-forming vaccine for potentiating an immune response against an antigen, wherein at least one dose of the depot-forming vaccine comprising the antigen and a hydrophobic carrier is for administration prior to the non-depot-forming vaccine comprising the antigen.

(77) The use according to paragraph (76), wherein the depot-forming vaccine primes the immune response to the antigen, and subsequent administration of the non-depot-forming vaccine maintains and/or boosts the immune response to the antigen.

(78) The use according to paragraph (76) or (77), wherein the non-depot-forming vaccine is cleared from an injection site faster than the depot-forming vaccine.

(79) The use according to any one of paragraphs (76) to (78), wherein potentiation of the immune response comprises a reduction in the occurrence of injection site reactions.

(80) The use according to any one of paragraphs (76) to (79), wherein the depot-forming vaccine is as defined in any one of paragraphs (25) to (46), (49), (50), (62), (63), (64) and (66) and the non-depot-forming vaccine is as defined in any one of paragraphs (47) to (50), (62), (63), (65) and (67).

(81) The use according to any one of paragraphs (76) to (80), wherein the depot-forming vaccine is as defined in paragraph (64) and the non-depot-forming vaccine is as defined in paragraph (65).

(82) The use according to any one of paragraphs (76) to (81), wherein the depot-forming vaccine is as defined in paragraph (66) and the non-depot-forming vaccine is as defined in paragraph (67).

(83) A kit comprising: at least one container comprising a depot-forming vaccine, said depot-forming vaccine vaccine comprising one or more antigens and a hydrophobic carrier; and at least one container comprising a non-depot-forming vaccine, said non-depot-forming vaccine vaccine comprising the one or more antigens.

(84) The kit of paragraph (83), wherein the non-depot-forming vaccine comprises an aqueous carrier.

(85) The kit of paragraph (83) or (84), wherein the depot-forming vaccine and non-depot-forming vaccine further comprise a T-helper epitope.

(86) The kit of paragraph (85), wherein the T-helper epitope is a peptide comprising the amino acid sequence FNNFTVSFWLRVPKVSASHLE (SEQ ID NO: 63).

(87) The kit of any one of paragraphs (83) to (86), wherein the depot-forming vaccine and non-depot-forming vaccine further comprise an adjuvant.

(88) The kit of paragraph (87), wherein the adjuvant is a polyl:C polynucleotide.

(89) The kit of any one of paragraphs (83) to (88), wherein the depot-forming vaccine and non-depot-forming vaccine further comprise lipids.

(90) The kit of paragraph (89), wherein the lipids are 1,2-dioleoyl-sn-glycero-3-phosphocholine (DOPC).

(91) A kit comprising: at least two containers, each container comprising one or more antigens, a T-helper epitope, an adjuvant and lipids; at least one container comprising a hydrophobic carrier; and at least one container comprising an aqueous carrier, wherein at least one container of antigen, T-helper epitope, adjuvant and lipids is for reconstitution with the hydrophobic carrier to prepare a depot-forming vaccine and at least one container of antigen, T-helper epitope, adjuvant and lipids is for reconstitution with the aqueous carrier to prepare a non-depot-forming vaccine.

(92) The kit of paragraph (91), wherein the T-helper epitope is a peptide comprising the amino acid sequence FNNFTVSFWLRVPKVSASHLE (SEQ ID NO: 63).

(93) The kit of paragraph (91) or (92), wherein the adjuvant is a polyl:C polynucleotide.

(94) The kit of any one of paragraphs (91) to (93), wherein the lipids are 1,2-dioleoyl-sn-glycero-3-phosphocholine (DOPC) and cholesterol.

(95) The kit of any one of paragraphs (83) to (94), wherein the one or more antigens comprise a mixture of five peptide antigens comprising the amino acid sequence FTELTLGEF (SEQ ID NO: 55); LMLGEFLKL (SEQ ID NO: 2); RISTFKNWPK (SEQ ID NO: 58); STFKNWPFL (SEQ ID NO: 59) or LPPAWQPFL (SEQ ID NO: 60).

(96) The kit of any one of paragraphs (83) to (94), wherein the antigen is a peptide antigen derived from human papillomavirus (HPV).

(97) The kit of any one of paragraphs (83) to (96), for use in potentiating an immune response in a subject by priming the immune response with the depot-forming vaccine and maintaining and/or boosting the immune response with the non-depot-forming vaccine.

(98) The kit of any one of paragraphs (83) to (97), wherein the depot-forming vaccine is water-free or substantially free of water.

(99) Combination of a depot-forming vaccine comprising one or more antigens and a hydrophobic carrier, and a non-depot-forming vaccine comprising the one or more antigens, for use in a method according to any one of paragraphs (1) to (75).

(100) Combination of a depot-forming vaccine comprising one or more antigens in a hydrophobic carrier and a non-depot-forming vaccine comprising the one or more antigens in an aqueous carrier, for use in a method for potentiating an immune response to an antigen in a subject, wherein the subject is administered at least one dose of the depot-forming vaccine; and the subject is subsequently administered at least one dose of the non-depot-forming vaccine.

The invention is further illustrated by the following non-limiting examples.

### EXAMPLES

### Example 1

Pathogen free, HHD-DR1 transgenic mice, 6-12 weeks of age, were bred in house, housed according to institutional guidelines with water and food ad libitum under filter controlled air circulation. The HHD-DR1 mice express human MHC class I and II molecules, HLA-A*0201 and HLA-DR*0101, and do not express corresponding murine MHC molecules, H-2D^{b}, -IA or -IE (β2m^{-/-}, H-2D^{b-/-}, IAα^{-/-}, IAβ^{-/-}, IEβ^{-/-}).

Mice were vaccinated with a mixture of five survivin peptide antigens formulated in an oil based depot forming vaccine or an aqueous based vaccine. Each survivin peptide antigen is restricted by different HLA (HLA-A1, A2, A3, A24 and B7). The vaccine formulations also contained a universal T-helper epitope derived from tetanus toxoid and a poly I:C polynucleotide adjuvant. To prepare vaccines, peptides and adjuvants were first reconstituted in acetate buffer (0.1 M, pH 9.5) with DOPC and cholesterol (Lipoid, Germany). The vaccine components were then lyophilized to form a dry cake. Just prior to injection, the dry cake was resuspended in ISA51 VG oil (SEPPIC, France) to prepare oil based formulations or water to prepare aqueous based formulations. The final vaccine formulation contained each survivin peptide antigen at 1 milligram per milliliter, T-helper at 500 micrograms per milliliter, adjuvant at 400 micrograms per milliliter, DOPC at 120 milligrams per milliliter and cholesterol at 12 milligrams per milliliter.

The immunogenicity of the vaccine formulations was tested by vaccinating HHD-DR1 transgenic mice. A 50 microliter dose of each vaccine was administered subcutaneously in the right flank of mice. Group 1 mice (n=5) were vaccinated with peptides and adjuvant formulated in the oil based formulation described above. Group 2 mice (n=5) were vaccinated with peptides and adjuvant formulated in the aqueous formulation described above. Vaccines were delivered subcutaneously on the right flank. Eight days after immunization, all mice were euthanized and right inguinal lymph nodes removed. A single cell suspension was prepared and lymph node cells were loaded into anti-IFN-gamma coated wells (200,000 cells per well) of an ELISPOT plate (BD Bioscience, San Jose, Calif.). Mature, syngeneic dendritic cells loaded with the HLA-A2 restricted survivin peptide (SurA2.M, LMLGEFLKL; SEQ ID NO: 2), an irrelevant HLA-A2 restricted peptide (ALMEQQHYV; SEQ ID NO: 1), or unloaded (background) were also added to the wells (20,000 cells per well). Cells were incubated in the ELISPOT plate for 18 hours. Next day, the plate was developed using AEC kit (Sigma, St. Louis, Mo.) and individual IFN-gamma secreting cells enumerated using an Immunospot plate reader (Cellular Technologies Ltd, Shaker Heights, Ohio). Results are shown in FIG. 1.

Mice in Group 1 generated an average response of 97 spot forming units (SFU) to stimulation with the SurA2.M peptide. Response to background and irrelevant peptide was negligible, <10 SFU. The response generated by SurA2.M stimulation was significantly higher than the response to irrelevant peptide by 2-way ANOVA, ***p<0.001.

Mice in Group 2 generated an average response of 2 SFU to stimulation with the SurA2.M peptide. Response to background and irrelevant peptide was negligible, <10 SFU. The response generated by SurA2.M stimulation was not significantly different than the response to irrelevant peptide by 2-way ANOVA, p>0.05.

These data demonstrate that administering an oil based vaccine is more efficacious than an aqueous based vaccine for inducing an antigen-specific immune response.

### Example 2

Pathogen free, HHD-DR1 transgenic mice, 6-12 weeks of age, were bred in house, housed according to institutional guidelines with water and food ad libitum under filter controlled air circulation. The HHD-DR1 mice express human MHC class I and II molecules, HLA-A*0201 and HLA-DR*0101, and do not express corresponding murine MHC molecules, H-2D^{b}, -IA or -IE (β2m^{-/-}, H-2D^{b-/-}, IAα^{-/-}, IEβ^{-/-}).

Mice were vaccinated with a mixture of five survivin peptide antigens formulated in an oil based depot forming vaccine or an aqueous based vaccine. Each survivin peptide antigen is restricted by different HLA (HLA-A1, A2, A3, A24 and B7). The vaccine formulations also contained a universal T-helper epitope derived from tetanus toxoid and a poly I:C polynucleotide adjuvant. To prepare vaccines, peptides and adjuvants were first reconstituted in acetate buffer (0.1M, pH 9.5) with DOPC and cholesterol (Lipoid, Germany). The vaccine components were then lyophilized to form a dry cake. Just prior to injection, the dry cake was resuspended in ISA51 VG oil (SEPPIC, France) to prepare oil based formulations or water to prepare aqueous based formulations. The final vaccine formulation contained each survivin peptide antigen at 1 milligram per milliliter, T-helper at 500 micrograms per milliliter, adjuvant at 400 micrograms per milliliter, DOPC at 120 milligrams per milliliter and cholesterol at 12 milligrams per milliliter.

Mice were also treated with metronomic cyclophosphamide (Sigma-Aldrich, St. Louis, Mo.) provided at a dose of 20 micrograms/kilogram/day by oral administration for 7 consecutive days.

The immunogenicity of the vaccine formulations was tested by vaccinating HHD-DR1 transgenic mice. Two groups of mice (n=5) were vaccinated with two 50 microliter immunizations of peptide antigens and adjuvant in the oil based formulation described above provided three weeks apart, on study days 0 and 21. On study day 84, nine weeks after the second vaccination, mice in group 1 were boosted with a 50 microliter immunization of the same oil based formulation. Mice in group 2 were boosted with a 50 microliter immunization of the aqueous based formulation. Throughout the study, mice in both groups were also treated with metronomic cyclophosphamide on alternating weeks, starting 7 days prior to the first immunization. Eight days following the boost immunization, on study day 92, all mice were euthanized and spleens removed. One naïve mouse was also terminated and served as a non-vaccinated control. A single cell suspension was prepared and splenocytes were loaded into anti-IFN-gamma coated wells (500,000 cells per well) of an ELISPOT plate (BD Bioscience, San Jose, Calif.). Cells were stimulated with 10 micrograms per milliliter of the HLA-A2 restricted survivin peptide (SurA2.M, LMLGEFLKL; SEQ ID NO: 2), an irrelevant HLA-A2 restricted peptide (ALMEQQHYV; SEQ ID NO: 1), or media containing no peptide (background). Cells were incubated in the ELISPOT plate for 18 hours. Next day, the plate was developed using AEC kit (Sigma, St. Louis, Mo.) and individual IFN-gamma secreting cells enumerated using an Immunospot plate reader (Cellular Technologies Ltd, Shaker Heights, Ohio). Results are shown in FIG. 2.

Mice in group 1 generated an average response of 423 spot forming units (SFU) to stimulation with the SurA2.M peptide. Response to background and irrelevant peptide was negligible, <10 SFU. The response generated by SurA2.M stimulation was significantly higher than the response to irrelevant peptide by 2-way ANOVA, ****p<0.0001.

Mice in group 2 generated an average response of 147 SFU to stimulation with the SurA2.M peptide. Response to background and irrelevant peptide was negligible, <10 SFU. The response generated by SurA2.M stimulation was significantly higher than the response to irrelevant peptide by 2-way ANOVA, *p<0.05.

These data demonstrate that an aqueous based vaccine can induce an antigen-specific recall response in mice that have been previously primed by immunization with an oil based vaccine.

### Example 3

Pathogen free, C57BL/6NCrl mice, 6-8 weeks of age, were purchased from Charles River Laboratories (St. Constant, PQ) and housed according to institutional guidelines with water and food ad libitum under filter controlled air circulation.

Mice were vaccinated with the HPV16E7₄₉₋₅₇ peptide antigen (R9F; RAHYNIVTF; SEQ ID NO: 3), a universal T helper epitope derived from tetanus toxin₉₄₇₋₉₆₇ (F21E; FNNFTVSFWLRVPKVSASHLE; SEQ ID NO: 63) and a poly I:C polynucleotide adjuvant. The peptides and adjuvant were formulated in an oil based depot forming vaccine or an aqueous based vaccine. To prepare the vaccines, peptides were first reconstituted in 30% tert-butanol with DOPC and cholesterol (Lipoid, Germany). To this antigen-lipid mixture, adjuvant was added and lyophilized to form a dry cake. Just prior to injection, the dry cake was resuspended in ISA51 VG oil (SEPPIC, France) to prepare oil based formulations or water to prepare aqueous based formulations. Each dose contained 5 micrograms of R9F peptide, 5 micrograms of F21E peptide, 20 micrograms of adjuvant, 6 milligrams of DOPC and 0.6 milligrams of cholesterol.

Mice were also treated with metronomic cyclophosphamide (Sigma-Aldrich, St. Louis, Mo.) provided at a dose of 20 micrograms/ kilogram/ day by oral administration for 7 consecutive days.

The immunogenicity of the vaccine formulations was tested by vaccinating three groups of mice (n=5). Mice in group 1 were vaccinated on days 0, 21, 42 with oil based vaccine formulation. Mice in group 2 were vaccinated on days 0 and 21 with the oil based vaccine formulation and on day 42 with the aqueous formulation. Mice in group 3 were vaccinated on day 0 with the oil based formulation and on days 21 and 42 with the aqueous formulation. Cyclophosphamide was provided to all groups of mice for alternating one week on and one week off, starting 7 days prior to the first immunization (day 0). All mice were terminated on day 50 and spleens removed for analysis. A single cell suspension was prepared and splenocytes were loaded into anti-IFN-gamma coated wells (500,000 cells per well) of an ELISPOT plate (BD Bioscience, San Jose, Calif.). Cells were stimulated with 10 micrograms per milliliter of the HPV16E7₄₉₋₅₇ peptide (R9F, RAHYNIVTF; SEQ ID NO: 3), an irrelevant H-2b restricted peptide (RMFPNAPYL; SEQ ID NO: 4), or media containing no peptide (background). Cells were incubated in the ELISPOT plate for 18 hours. Next day, the plate was developed using AEC kit (Sigma, St. Louis, Mo.) and individual IFN-gamma secreting cells enumerated using an Immunospot plate reader (Cellular Technologies Ltd, Shaker Heights, Ohio). Results are shown in FIG. 3.

Mice in group 1 generated an average response of 177 spot forming units (SFU) to stimulation with the R9F peptide. Response to background and irrelevant peptide was negligible, <10 SFU.

Mice in group 2 generated an average response of 127 spot forming units (SFU) to stimulation with the R9F peptide. Response to background and irrelevant peptide was negligible, <10 SFU.

Mice in group 3 generated an average response of 194 spot forming units (SFU) to stimulation with the R9F peptide. Response to background and irrelevant peptide was negligible, <10 SFU.

The response generated by R9F stimulation was not significantly different between each group by 1-way ANOVA.

These data demonstrate that an aqueous vaccine can induce a systemic recall response primed with one or two immunizations with an oil based vaccine formulation and that boosting with an aqueous vaccine containing the same antigen can maintain such an immune response.

### Example 4

Pathogen free, C57BL/6NCrl mice, 6-8 weeks of age, were purchased from Charles River Laboratories (St. Constant, PQ) and housed according to institutional guidelines with water and food ad libitum under filter controlled air circulation.

Mice were vaccinated with the HPV16E7₄₉₋₅₇ peptide antigen (R9F; RAHYNIVTF; SEQ ID NO: 3), a universal T helper epitope derived from tetanus toxin₉₄₇₋₉₆₇ (F21E; FNNFTVSFWLRVPKVSASHLE; SEQ ID NO: 63) and a poly I:C polynucleotide adjuvant. The peptides and adjuvant were formulated in an oil based depot forming vaccine or an aqueous based vaccine. To prepare the vaccines, peptides were first reconstituted in 30% tert-butanol with DOPC and cholesterol (Lipoid, Germany). To this antigen-lipid mixture, adjuvant was added and lyophilized to form a dry cake. Just prior to injection, the dry cake was resuspended in ISA51 VG oil (SEPPIC, France) to prepare oil based formulations or water to prepare aqueous based formulations. Each dose contained 5 micrograms of R9F peptide, 5 micrograms of F21E peptide, 20 micrograms of adjuvant, 6 milligrams of DOPC and 0.6 milligrams of cholesterol.

Mice were also treated with metronomic cyclophosphamide (Sigma-Aldrich, St. Louis, Mo.) provided at a dose of 20 micrograms/kilogram/day by oral administration for 7 consecutive days.

Efficacy of the treatment was tested in mice bearing HPV16E7-expressing C3 tumors. Tumors were implanted subcutaneously on study day 0 and growth monitored weekly. Palpable tumors were measured twice weekly using calipers to record the length and width. Mice were humanly euthanized when tumor volume reached predefined endpoint volume of 2000 mm³. Mice in group 1 remained untreated and served as the tumor growth control group (n=9). Mice in groups 2-4 (n=10) were treated with metronomic cyclophosphamide starting 5 days after tumor cell implantation and were vaccinated on days 12, 33, 54. Mice in group 2 were vaccinated with oil based vaccine on days 12, 33 and 54. Mice in group 3 were vaccinated with oil based vaccine on days 12 and 33 and aqueous vaccine on day 54. Mice in group 4 were vaccinated with oil based vaccine on day 12 and aqueous vaccine on days 33 and 54. Results are shown in FIG. 4.

FIG. 4A shows the average tumor volume recorded for each group. The majority of mice in group 1 were terminated due to large tumor volume or tumor ulceration by study day 29, the average tumor volume at this point was 468±41 mm³. On study day 49, the average tumor volume of group 2 was 281±99 mm³, the average tumor volume of group 3 was 226±75 mm³, and the average tumor volume of group 4 was 327±103 mm³. Statistical analysis was performed by linear regression and no significant differences were detected between groups 2, 3 and 4.

FIG. 4B shows the percent survival of each group. By study day 64, 0% of the mice in group 1 were remaining, 60% of the mice in group 2 were remaining, 50% of the mice in group 3 were remaining, and 80% of the mice in group 4 were remaining. Statistical analysis was performed by Mantel-Cox and no significant differences were detected between groups 2, 3 and 4.

This data demonstrates that priming with an oil based vaccine and then boosting with aqueous vaccine containing the same antigen can provide effective protection from tumor growth.

### Example 5

Pathogen free, HHD-DR1 transgenic mice, 6-12 weeks of age, were bred in house, housed according to institutional guidelines with water and food ad libitum under filter controlled air circulation. The HHD-DR1 mice express human MHC class I and II molecules, HLA-A*0201 and HLA-DR*0101, and do not express corresponding murine MHC molecules, H-2D^{b}, -IA or -IE (β2m^{-/-}, H-2D^{b-/-}, IAα^{-/-}, IEβ^{-/-}).

Mice were vaccinated with a mixture of five survivin peptide antigens formulated in an oil based depot forming vaccine. Each survivin peptide antigen is restricted by different HLA (HLA-A1, A2, A3, A24 and B7). The vaccine formulations also contained a universal T-helper epitope derived from tetanus toxoid and a poly I:C polynucleotide adjuvant. To prepare vaccines, peptides and adjuvant were first reconstituted in sodium acetate buffer (0.1 M, pH 9.5) with DOPC and cholesterol (Lipoid, Germany). The vaccine components were then lyophilized to form a dry cake. Just prior to injection, the dry cake was resuspended in ISA51 VG oil (SEPPIC, France). The final vaccine formulation contained each survivin peptide antigen at 1 milligram per milliliter, T-helper at 500 micrograms per milliliter, adjuvant at 400 micrograms per milliliter, DOPC at 120 milligrams per milliliter and cholesterol at 12 milligrams per milliliter.

Vaccine immunogenicity was tested by vaccinating HHD-DR1 transgenic mice with a 50 microliter dose of the oil based vaccine subcutaneously in the flank. Mice in group 1 (n=5) were vaccinated once on study day 0 and terminated on day 8. Mice in group 2 (n=5) were vaccinated twice on study days 0 and 21 and terminated on day 28 (8 days after second immunization). Mice in group 3 were vaccinated twice on study days 0 and 21 and terminated on day 48 (27 days after second immunization). Upon termination spleens were removed. A single cell suspension was prepared from the spleens and splenocytes were loaded into anti-IFN-gamma coated wells (500,000 cells per well) of an ELISPOT plate (BD Bioscience, San Jose, Calif.). Cells were stimulated with 10 micrograms per milliliter of the HLA-A2 restricted survivin peptide (SurA2.M, LMLGEFLKL; SEQ ID NO: 2), an irrelevant HLA-A2 restricted peptide (ALMEQQHYV; SEQ ID NO: 1), or media containing no peptide (background). Cells were incubated in the ELISPOT plate for 18 hours. Next day, the plate was developed using AEC kit (Sigma, St. Louis, Mo.) and individual IFN-gamma secreting cells enumerated using an Immunospot plate reader (Cellular Technologies Ltd, Shaker Heights, Ohio). Results are shown in FIG. 5.

Mice in Group 1 generated an average response of 131 spot forming units (SFU) to stimulation with the SurA2.M peptide. Response to background and irrelevant peptide was negligible, <10 SFU.

Mice in Group 2 generated an average response of 351 SFU to stimulation with the SurA2.M peptide. Response to background and irrelevant peptide was negligible, <10 SFU.

Mice in Group 3 generated an average response of 49 SFU to stimulation with the SurA2.M peptide. Response to background and irrelevant peptide was negligible, <10 SFU.

The response generated by SurA2.M stimulation was significantly different between groups 1 and 2, p<0.05. As expected, boosting with a second vaccine dose maintained and increase the immune response (group 1 versus group 2). The response generated by SurA2.M stimulation was significantly different between groups 2 and 3, p<0.01, demonstrating that the immune response dropped significantly within 27 days following the administration of a booster vaccination. These data demonstrate that an antigen-specific response induced by a vaccine declines over time without boosting and highlights the importance of administering booster immunizations to maintain such an immune response, particularly a cellular immune response. Statistical analysis performed by 1-way ANOVA with Tukey post-test.

### Example 6

A phase 1b study, designated ONC-DPX-Survivac-03, conducted in the United States (IND #14731) and Canada (CTA-A #183600) contained a cohort of subjects that examined the safety and immune potency of priming with an oil-based vaccine formulation (depot-forming) followed by boosting with an aqueous-based vaccine formulation (non-depot-forming) along with low dose cyclophosphamide in ovarian cancer subjects.

The vaccine used in this study was DPX-Survivac which consists of five human leukocyte antigen (HLA)-restricted epitopes (HLA-Al: FTELTLGEF (SEQ ID NO: 55), HLA-A2: LMLGEFLKL (SEQ ID NO: 2), HLA-A3: RISTFKNWPK (SEQ ID NO: 58), HLA-A24: STFKNWPFL (SEQ ID NO: 59), and HLA-B7: LPPAWQPFL (SEQ ID NO: 60)), a universal T-helper epitope from tetanus toxoid (AQYIKANSKFIGITEL; SEQ ID NO: 61), a poly I:C polynucleotide adjuvant, and liposomes consisting of DOPC and cholesterol. The antigen/adjuvant/liposome complex is formulated in a phosphate buffer, filled into vials and lyophilized to a dry cake. In the clinic, the cake is re-suspended in 1× volume of the hydrophobic carrier Montanide ISA51 VG (SEPPIC, France) before injection to prepare the oil-based formulation. To prepare the aqueous-based formulation, the dry cake is re-suspended using 2× volume of sterile water instead of a hydrophobic carrier. The dose volumes and deliverables in the final vaccine formulations are shown in Table 4.

TABLE 4
Components of each dose of an oil-based DPX-Survivac
vaccine and an aqueous-based DPX-Survivac vaccine.

| | | |
|---|---|---|
| Component | DPX-Survivac (Oil) | DPX-Survivac (Aqueous) |

| | | |
|---|---|---|
| Diluent | Montanide ISA51 VG | Sterile water |

| | | | | |
|---|---|---|---|---|
| Dose volume | 0.250 | milliliters | 0.500 | milliliters |
| Antigens | 0.250 | milligrams | 0.250 | milligrams |
| T-helper | 0.125 | milligrams | 0.125 | milligrams |
| Adjuvant | 0.1 | milligrams | 0.1 | milligrams |
| DOPC | 30 | milligrams | 30 | milligrams |
| cholesterol | 3 | milligrams | 3 | milligrams |

In the clinical study, subjects received two subcutaneous priming injections of 0.25 milliliter dose of DPX-Survivac (Oil) and three subcutaneous boosting injections of 0.50 milliliter dose of DPX-Survivac (Aqueous), 4 weeks apart. Subjects were also treated with oral cyclophosphamide at 50 milligrams BID (two times a day) on alternating weeks for the duration of the study. The clinical trial was conducted in accordance with the schedule shown in FIG. 6.

Adverse events were assessed according to the National Cancer Institute Common Terminology Criteria for Adverse Events (CTCAE) version 4.03. Blood was collected prior to the first vaccination (baseline), at each vaccination time point, and two weeks after the second priming vaccination (study days 0, 28, 42, 56, 84, 112). Peripheral blood mononuclear cells (PBMCs) were isolated and cryo-preserved to study immune function and vaccine-induced T cell immunity by (i) Interferon-gamma ELISPOT and (ii) multimer analysis.

(i) Interferon-Gamma ELISPOT Assay for Detecting Functional Antigen-Specific T Cells in PBMC

Antigen-specific T cells generated in response to vaccination secrete the cytokine interferon gamma (IFN-gamma) when they encounter cognate peptide. The ELISPOT assay can measure the secretion of IFN-gamma and quantify the number of cells that are secreting it in response to stimulation with peptide.

PBMC samples collected from clinical trial subjects were tested in the interferon-gamma (IFN-gamma) ELISPOT assay for a recall response to the specified test antigens. The ELISPOT was performed using a kit purchased from C.T.L. Ltd. (Shaker Heights, Ohio, USA). PBMC were thawed, and stimulated with a pool of five survivin peptides. The responses to the antigens, negative control (cells in medium alone) and the positive control (T cell activators PMA and Ionomycin) were tested in duplicate wells. The PBMC from clinical samples were plated at a concentration of 300,000 cells/well of a 96-well ELISPOT plate. PBMC from a healthy control subject were also used as a control.

On day 1, plates were coated with capture antibody (purified anti-IFN-gamma) diluted 1:250 in phosphate buffered saline (PBS) and the plates were incubated overnight in a 2-6° C. fridge. On day 2, plates were washed once with PBS; washing was performed by adding 200 microliters per well of buffer followed by inverting the plates and flicking out the contents over a sink. The antigens were prepared in serum free CTL-Test™ media (C.T.L. Ltd) at a concentration of 100 micrograms per milliliter each. PBMCs were re-suspended in serum free CTL-Test™ media at a concentration of 3×10E6 cells per milliliter. Peptides and cells were each added to the wells at 100 microliters per well each, for a final volume of 200 microliters. Plates were incubated in a humidified 37° C., 5% CO₂ incubator overnight. On day 3, plates were developed using Human IFN-gamma ELISPOT set reagents purchased from C.T.L Ltd. Plates were washed twice with PBS, twice with 0.05% Tween20-PBS followed by addition of 100 microliters per well of detection antibody (biotinylated anti-IFN-gamma). After 2 hours incubation at room temperature (18-23° C.), plates were washed three times with 0.05% Tween20-PBS, followed by adding 100 microliter per well of streptavidin-alkaline phosphatase reagent and incubated at room temperature for 2 hours. Plates were washed twice with 0.05% Tween20-PBS and twice with PBS and then 100 microliters of freshly prepared TrueBlue peroxidase substrate developer solution added. Spot development was monitored at room temperature for 15-20 minutes, to confirm appearance of spots in antigen containing wells. The reaction was stopped by rinsing plates with tap water. Plates were air dried overnight and stored at room temperature. The number of spots within the wells of the ELISPOT plates were enumerated using an automated plate analyzer (ImmunoSpot S6 Core Analyzer, C.T.L. LTd.). Data is expressed as spot forming unit (SFU) counts per million PBMCs which is obtained by averaging the SFU for each duplicate well then multiplying by 3.33.

(ii) Multimer Staining

Antigen-specific CD8+ T cells mediate immune responses by recognizing specific peptide presented in MHC molecules on the surface of antigen presenting cells. They do this through their T cell receptor (TCR). Multimer reagents can be used in conjunction with immunofluorescence analysis to quantify the percent of all CD8+ T cells which bear antigen-specific TCR. This assay was performed using PBMCs and fluorescent conjugated multimer reagents and antibodies, which were detected by flow cytometry.

PBMCs were thawed and re-suspended in serum-free CTL-Test™ media at a concentration of 1×10E7. Cells were cultured overnight in a 37° C., 5% CO₂ incubator. The next day, cell viability was assessed using trypan blue exclusion staining. Cells were divided into assay tubes and stained with PE-conjugated multimer reagents in buffer (PBS+2% fetal bovine serum; FBS) for 30 minutes at 4° C. in the dark. Cells were then washed by adding 1 milliliter of PBS+2% FBS and centrifuging at 300×g for 5 minutes. Supernatant was discarded and the cell pellet re-suspended in 100 microliters of PBS+2% FBS and stained with anti-human-CD3-APC (OKT3) and anti-human-CD8alpha-FITC (OKT8) for 30 minutes at 4° C. in the dark. Cells were then washed as above and the pellet re-suspended in 100 microliters of PBS+2% FBS. Fifteen minutes prior to acquisition on a flow cytometer, 5 microliters of 7-AAD viability dye was added to each sample. Data was acquired using a

FACSCalibur (BD Bioscience) and analysed using WinList 6.0 (Verity Software House). Gates were set to exclude non-viable cells (7-AAD+), then to include CD3+ and CD8+ cells.

The antigen specific immune response of two subjects enrolled in this study, as assessed by IFN-gamma ELISPOT and tetramer analysis are described in Examples 7 and 8.

### Example 7

Subject 03-28 is a 66 year old, HLA-A2 positive female diagnosed with stage 3c ovarian cancer 14 months prior to inclusion in the study ONC-DPX-Survivac-03. At the time of diagnosis, the subject underwent standard debulking surgery followed by one course of platinum and taxol based chemotherapy. The subject did not exhibit recurrence prior to enrollment in this clinical study, and treatment was initiated 9 months after completion of standard of care.

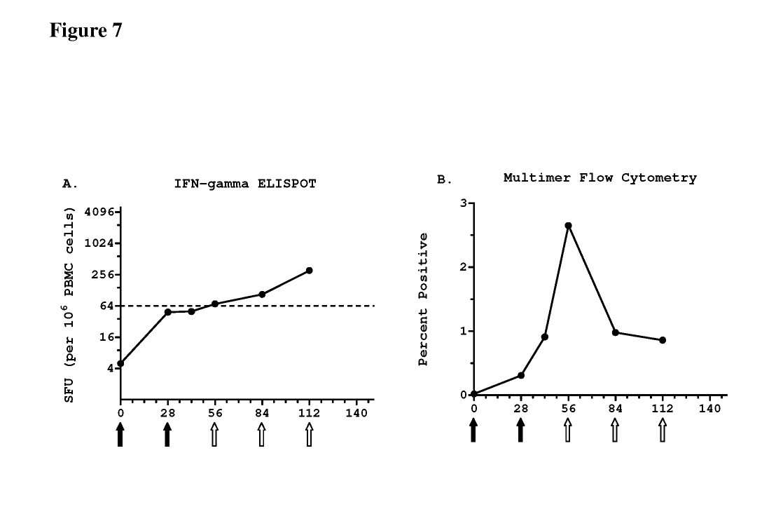

Immune responses were monitored using subject PBMCs isolated from blood collected at study days 0, 28, 42, 56, 84 and 112 by IFN-gamma ELISPOT and multimer flow cytometry as described in Example 6. Results are shown in FIG. 7. ELISPOT responses rose gradually after priming vaccinations with DPX-Survivac (Oil) were complete, and appear to be increased after boosting vaccinations with DPX-Survivac (Aqueous). Circulating SurA2.M-specific CD8+ T cells detected by multimer flow cytometry peaked at study day 56 and were maintained above background until study day 112.

These results demonstrate that DPX-Survivac (Oil) is capable of priming an immune response in a HLA-A2+ subject with no prior immune response towards the vaccine antigen. Boosting with DPX-Survivac (Aqueous) maintained these immune responses.

### Example 8

Subject 03-30 is a 52 year old, HLA-A1 positive subject diagnosed with stage 3a ovarian cancer 13 months prior to inclusion in study ONC-DPX-Survivac-03. At the time of diagnosis, the subject underwent standard of care debulking surgery followed by one course of platinum and taxol based chemotherapy and experienced a complete response. The subject did not exhibit recurrence prior to enrollment in this clinical study, and treatment was initiated 8 months after completion of standard of care.

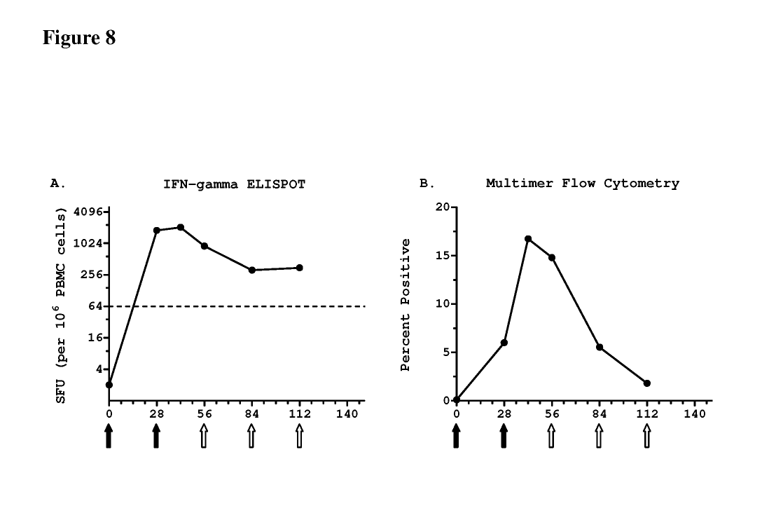

Immune responses were monitored using subject PBMCs isolated from blood collected at study days 0, 28, 42, 56, 84 and 112 by IFN-gamma ELISPOT and multimer flow cytometry as described in Example 6. Results are shown in FIG. 8. ELISPOT responses rose rapidly during the priming vaccinations with DPX-Survivac (Oil) and were maintained at elevated levels for the duration of the boosting immunizations with DPX-Survivac (Aqueous). Circulating SurA1.T specific T cells detected by multimer flow cytometry peaked at study day 42, two weeks following priming immunization with DPX-Survivac (Oil), and were maintained at levels above background for the duration of boosting immunizations with DPX-Survivac (Aqueous).

These results demonstrate that DPX-Survivac (Oil) is capable of priming an immune response in a HLA-A1+ subject with no prior immune response towards the vaccine antigen. Boosting with DPX-Survivac (Aqueous) maintained these immune responses.

In the patients outlined in the examples who received DPX-Survivac (Aqueous) formulations after DPX-Survivac (Oil) priming doses, all injection site reactions were mild (grade 1) and consisted of erythema, pruritis, induration and pain. The majority of these site reactions were completely resolved two to three months later, particularly if the injection site received the aqueous formulation.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

### REFERENCES

1) Cox J C, Drane D P, Suhrbier A. Immunogenic complexes and method relating thereto. CSL Ltd. EP1150710B1, filed 2000 Feb. 17, published 2010 Mar. 21.
2) Hayman W A, Toth I, Flinn N, Scanlon M, Good M F. Enhancing the immunogenicity and modulating the fine epitope recognition of antisera to a helical group A streptococcal peptide vaccine candidate from the M protein using lipid-core peptide technology. Immunol Cell Biol. 2002 April; 80(2):178-87.
3) Irvine D J, Swartz M A, Szeto G L. Engineering synthetic vaccines using cues from natural immunity. Nat Mater. 2013 November; 12(11):978-90. doi: 10.1038/nmat3775.
4) Karkada M, Weir G M, Quinton T, Sammatur L, MacDonald L D, Grant A, Liwski R, Juskevicius R, Sinnathamby G, Philip R, Mansour M. A novel breast/ovarian cancer peptide vaccine platform that promotes specific type-1 but not Treg/Tr1-type responses. J Immunother. 2010 April; 33(3):250-61. doi: 10.1097/CJI.0b013e3181c1f1e9.
5) Karpen H E, Bukowski J T, Hughes T, Gratton J P, Sessa W C, Gailani M R. The sonic hedgehog receptor patched associates with caveolin-1 in cholesterol-rich microdomains of the plasma membrane. J Biol Chem. 2001 Jun. 1; 276(22):19503-11. Epub 2001 Mar. 1.
6) Kim R, Emi M, Tanabe K. Cancer immunoediting from immune surveillance to immune escape. Immunology. 2007 May; 121(1):1-14. Epub 2007 Mar. 26.
7) Kovjazin R, Volovitz I, Daon Y, Vider-Shalit T, Azran R, Tsaban L, Carmon L, Louzoun Y. Signal peptides and trans-membrane regions are broadly immunogenic and have high CD8+ T cell epitope densities: Implications for vaccine development. Mol Immunol. 2011 April; 48(8):1009-18. doi: 10.1016/j.molimm.2011.01.006. Epub 2011 Feb. 12.
8) Moyle P M, Toth I. Self-adjuvanting lipopeptide vaccines. Curr Med Chem. 2008; 15(5):506-16.
9) Resh M D. Fatty acylation of proteins: new insights into membrane targeting of myristoylated and palmitoylated proteins. Biochim Biophys Acta. 1999 Aug. 12; 1451(1):1-16.
10) Robinson J H, Case M C, Brooks C G. Palmitic acid conjugation of a protein antigen enhances major histocompatibility complex class II-restricted presentation to T cells. Immunology. 1992 August; 76(4):593-8.
11) Sabbatini P, Tsuji T, Ferran L, Ritter E, Sedrak C, Tuballes K, Jungbluth A A, Ritter G, Aghajanian C, Bell-McGuinn K, Hensley M L, Konner J, Tew W, Spriggs D R, Hoffman E W, Venhaus R, Pan L, Salazar A M, Diefenbach C M, Old L J, Gnjatic S. Phase I trial of overlapping long peptides from a tumor self-antigen and poly-ICLC shows rapid induction of integrated immune response in ovarian cancer patients. Clin Cancer Res. 2012 Dec. 1; 18(23):6497-508. doi: 10.1158/1078-0432.CCR-12-2189. Epub 2012 Oct. 2.
12) Smotrys J E, Linder M E. Palmitoylation of intracellular signaling proteins: regulation and function. Annu Rev Biochem. 2004; 73:559-87.
13) Stills H F Jr. Adjuvants and antibody production: dispelling the myths associated with Freund's complete and other adjuvants. ILAR J. 2005; 46(3):280-93.
14) Tsuji T, Sabbatini P, Jungbluth A A, Ritter E, Pan L, Ritter G, Ferran L, Spriggs D, Salazar A M, Gnjatic S. Effect of Montanide and poly-ICLC adjuvant on human self/tumor antigen-specific CD4+ T cells in phase I overlapping long peptide vaccine trial. Cancer Immunol Res. 2013 November; 1(5):340-50. doi: 10.1158/2326-6066.CIR-13-0089. Epub 2013 Sep. 16.

15) Yoo H S, Choi H K, Park T G. Protein-fatty acid complex for enhanced loading and stability within biodegradable nanoparticles. J Pharm Sci. 2001 February; 90(2):194-201.
16) Zhang F L, Casey P J. Protein prenylation: molecular mechanisms and functional consequences. Annu Rev Biochem. 1996; 65:241-69.

1. A method for potentiating an immune response to an antigen in a subject, said method comprising:
(i) administering to the subject at least one dose of a depot-forming vaccine comprising one or more antigens in a hydrophobic carrier; and
(ii) subsequently administering to the subject at least one dose of a non-depot-forming vaccine comprising the one or more antigens.

2. The method according to claim 1, wherein:
each of the at least one dose of the depot-forming vaccine is a priming dose that is capable of inducing an immune response to the one or more antigens; and
each of the at least one dose of the non-depot-forming vaccine is a maintenance or boosting dose that is capable of maintaining and/or boosting the immune response to the one or more antigens.

3. (canceled)

4. The method according to claim 2, which comprises administering a first maintenance or boosting dose of the non-depot-forming vaccine within about 1 day, 1 week, 2 weeks, 3 weeks, 4 weeks, 5 weeks, 6 weeks, 7 weeks, 8 weeks, 9 weeks or 10 weeks of a final priming dose of the depot-forming vaccine.

5. (canceled)

6. The method according to claim 1, wherein the at least one dose of the depot-forming vaccine is one, two, three, four or five doses.

7. (canceled)

8. The method according to claim 1, wherein:
the at least one dose of the non-depot-forming vaccine is one, two, three, four or five doses; or
the at least one dose of the non-depot-forming vaccine is a continuous repeated dosing once every day, once every week, once every two weeks, once every three weeks, or once monthly.

9. (canceled)

10. The method according to claim 1, wherein:
after a first dose of the depot-forming vaccine, each subsequent dose of the depot-forming vaccine is administered within about 1 day, 1 week, 2 weeks, 3 weeks, or 4 weeks of the immediately preceding dose; and/or
after a first dose of the non-depot-forming vaccine, each subsequent dose of the non-depot-forming vaccine is administered within about 1 day, 1 week, 2 weeks, 3 weeks, or 4 weeks of the immediately preceding dose.

11. The method according to claim 10, wherein each subsequent dose of the depot-forming vaccine and non-depot-forming vaccine is administered within about 3 weeks or about 4 weeks of the immediately preceding dose.

12. The method according to claim 10, which comprises administering one or two doses of the depot-forming vaccine prior to administration of the non-depot-forming vaccine.

13. (canceled)

14. (canceled)

15. The method according to claim 1, which comprises administering the depot-forming vaccine on day 0 and day 21, and administering the non-depot-forming vaccine on day 42.

16. The method according to claim 1, which comprises administering the depot-forming vaccine on day 0, and administering the non-depot-forming vaccine on day 21 and day 42.

17. (canceled)

18. The method according to claim 1, which further comprises administering to the subject an agent that interferes with DNA replication and/or an immune response checkpoint inhibitor.

19. The method according to claim 18, which comprises administering the agent that interferes with DNA replication and wherein the agent that interferes with DNA replication is cyclophosphamide.

20. The method according to claim 19, which comprises a cycle of low dose metronomic cyclophosphamide, wherein the cycle comprises administering the cyclophosphamide to the subject daily for a period of 7 consecutive days, beginning every second week.

21. (canceled)

22. The method according to claim 20, wherein the cyclophosphamide is first administered 7 days prior to the first administration with the depot-forming vaccine.

23. (canceled)

24. (canceled)

25. The method according to claim 1, wherein the hydrophobic carrier of the depot-forming vaccine is an oil or a mixture of oils.

26. (canceled)

27. The method according to claim 25, wherein the hydrophobic carrier is mineral oil or is a mannide oleate in mineral oil solution.

28. The method according to claim 25, wherein the depot-forming vaccine is water-free or is substantially free of water.

29. (canceled)

30. The method according to claim 25, wherein the one or more antigens are sufficiently hydrophobic, or are made sufficiently hydrophobic, such that the one or more antigens are miscible in the oil.

31. The method according to claim 30, wherein the one or more antigens are naturally hydrophobic or are made sufficiently hydrophobic by the presence of an amphiphile in the depot-forming vaccine.

32-40. (canceled)

41. The method according to claim 31, wherein:
the amphiphile is closely associated with the one or more antigens to make the one or more antigens miscible in the hydrophobic carrier; and/or
the amphiphile forms a sheet or vesicular structure, partially or completely surrounding the one or more antigens.

42. (canceled)

43. The method according to claim 41, wherein the amphiphile is a lipid.

44. The method according to claim 43, wherein the lipids form a closed vesicular structure around the one or more antigens.

45. The method according to claim 44, wherein the closed vesicular structure is a reverse micelle.

46. (canceled)

47. The method according to claim 1, wherein the non-depot-forming vaccine comprises an aqueous carrier.

48. (canceled)

49. The method according to claim 1, wherein the depot-forming vaccine and/or the non-depot-forming vaccine further comprise an adjuvant and/or a T-helper epitope.

50. The method according to claim 49, wherein the adjuvant is a polyI:C polynucleotide and the T-helper epitope comprises the amino acid sequence AQYIKANSKFIGITEL (SEQ ID NO: 61) or FNNFTVSFWLRVPKVSASHLE (SEQ ID NO: 63).

51. The method according to claim 1, wherein the one or more antigens are: (i) derived from a virus, bacterium or protozoan; (ii) a membrane surface-bound cancer antigen; or (iii) a toxin.

52. (canceled)

53. The method according to claim 1, wherein the antigen is a survivin antigen.

54. The method according to claim 53, wherein the survivin antigen is a peptide antigen comprising an amino acid sequence from the survivin protein (SEQ ID NO: 53) or a variant thereof, or a nucleic acid molecule encoding said peptide antigen.

55. The method according to claim 1, wherein the one or more antigens comprise one or more peptide antigens comprising an amino acid sequence selected from the group consisting of FEELTLGEF (SEQ ID NO: 54); FTELTLGEF (SEQ ID NO: 55); LTLGEFLKL (SEQ ID NO: 56); LMLGEFLKL (SEQ ID NO: 2); RISTFKNWPF (SEQ ID NO: 57); RISTFKNWPK (SEQ ID NO:58); STFKNWPFL (SEQ ID NO: 59); and LPPAWQPFL (SEQ ID NO: 60), or any combination thereof; or one or more nucleic acid molecules encoding said one or more peptide antigens.

56. The method according to claim 1, wherein the one or more antigens comprise a mixture of five peptide antigens, the first peptide antigen comprises the amino acid sequence FTELTLGEF (SEQ ID NO: 55); the second peptide antigen comprises the amino acid sequence LMLGEFLKL (SEQ ID NO: 2); the third peptide antigen comprises the amino acid sequence RISTFKNWPK (SEQ ID NO: 58); the fourth peptide antigen comprises the amino acid sequence STFKNWPFL (SEQ ID NO: 59) and the fifth peptide antigen comprises the amino acid sequence LPPAWQPFL (SEQ ID NO: 60).

57. The method according to claim 1, wherein the antigen is a peptide antigen derived from human papillomavirus (HPV) or a nucleic acid molecule encoding said peptide antigen.

58. The method according to claim 57, wherein the peptide antigen derived from HPV comprises the amino acid sequence YMLDLQPETT (SEQ ID NO: 44), YMLDLQPET (SEQ ID NO: 45); LLMGTLGIV (SEQ ID NO: 46) or TLGIVCPI (SEQ ID NO: 47).

59. The method according to claim 1, wherein the antigen is a self-antigen; a cancer-associated antigen and/or a weakly immunogenic antigen.

60-63. (canceled)

64. The method according to claim 1, wherein the depot-forming vaccine comprises:
(i) five survivin peptide antigens, wherein the first peptide antigen comprises the amino acid sequence FTELTLGEF (SEQ ID NO: 55), the second peptide antigen comprises the amino acid sequence LMLGEFLKL (SEQ ID NO: 2), the third peptide antigen comprises the amino acid sequence RISTFKNWPK (SEQ ID NO: 58), the fourth peptide antigen comprises the amino acid sequence STFKNWPFL (SEQ ID NO: 59), and the fifth peptide antigen comprises the amino acid sequence LPPAWQPFL (SEQ ID NO: 60);
(ii) a T helper epitope comprising the amino acid sequence AQYIKANSKFIGITEL (SEQ ID NO: 61);
(iii) a polyI:C polynucleotide adjuvant;
(iv) a lipid molecule mixture of 1,2-dioleoyl-sn-glycero-3-phosphocholine (DOPC) and cholesterol lipid mixture; and
(v) the hydrophobic carrier Montanide® ISA 51 VG.

65. The method according to claim 64, wherein the non-depot-forming vaccine comprises the same components (i), (ii), (iii) and (iv), and a water carrier.

66. The method according to claim 1, wherein the depot-forming vaccine comprises:
(i) a peptide antigen derived from human papillomavirus (HPV);
(ii) a T helper epitope comprising the amino acid sequence AQYIKANSKFIGITEL (SEQ ID NO: 61);
(iii) a polyI:C polynucleotide adjuvant;
(iv) a lipid molecule mixture of 1,2-dioleoyl-sn-glycero-3-phosphocholine (DOPC) and cholesterol lipid mixture; and
(v) the hydrophobic carrier Montanide® ISA 51 VG.

67. The method according to claim 66, wherein the non-depot-forming vaccine comprises the same components (i), (ii), (iii) and (iv), and a water carrier.

68. The method according to claim 1, which is for potentiating a cytotoxic T-lymphocyte (CTL) immune response and/or an antibody immune response in the subject.

69. (canceled)

70. The method according to claim 1, wherein the subject has had no prior immune response towards the antigen.

71. The method according to claim 1, which is for treating or preventing cancer; an infectious disease; or an addiction disease.

72. (canceled)

73. (canceled)

74. The method according to claim 1, wherein the non-depot-forming vaccine is cleared from an injection site faster than the depot-forming vaccine.

75. The method according to claim 1, wherein potentiation of the immune response comprises a reduction in the occurrence of injection site reactions

76-99. (canceled)

100. The method according to claim 51, wherein the antigen is derived from Ebola virus, human papillomavirus (HPV), influenza virus, respiratory syncytial virus (RSV), Bordetella pertussis, Bacillus anthracis or Plasmodium malariae.


SEQUENCE LISTING (64 sequences)

SEQ ID NO: 1 - protein, 9 aa, Artificial Sequence
  Irrelevant HLA-A2 restricted peptide
ALMEQQHYV

SEQ ID NO: 2 - protein, 9 aa, Artificial Sequence
  Survivin HLA-A2 restricted peptide (modified)
LMLGEFLKL

SEQ ID NO: 3 - protein, 9 aa, Artificial Sequence
  HPV R9F peptide
RAHYNIVTF

SEQ ID NO: 4 - protein, 9 aa, Artificial Sequence
  Irrelevant peptide
RMFPNAPYL

SEQ ID NO: 5 - protein, 9 aa, Artificial Sequence
  Synthetic Peptide
AAGIGILTV

SEQ ID NO: 6 - protein, 10 aa, Artificial Sequence
  Synthetic Peptide
EAAGIGILTV

SEQ ID NO: 7 - protein, 9 aa, Artificial Sequence
  Synthetic Peptide
ILTVILGVL

SEQ ID NO: 8 - protein, 10 aa, Artificial Sequence
  Synthetic Peptide
AEEAAGIGIL

SEQ ID NO: 9 - protein, 11 aa, Artificial Sequence
  Synthetic Peptide
AEEAAGIGILT

SEQ ID NO: 10 - protein, 9 aa, Artificial Sequence
  Synthetic Peptide
TILLGIFFL

SEQ ID NO: 11 - protein, 9 aa, Artificial Sequence
  Synthetic Peptide
FLALIICNA

SEQ ID NO: 12 - protein, 9 aa, Artificial Sequence
  Synthetic Peptide
KTWGQYWQV

SEQ ID NO: 13 - protein, 10 aa, Artificial Sequence
  Synthetic Peptide
AMLGTHTMEV

SEQ ID NO: 14 - protein, 9 aa, Artificial Sequence
  Synthetic Peptide
MLGTHTMEV

SEQ ID NO: 15 - protein, 10 aa, Artificial Sequence
  Synthetic Peptide
SLADTNSLAV

SEQ ID NO: 16 - protein, 9 aa, Artificial Sequence
  Synthetic Peptide
ITDQVPFSV

SEQ ID NO: 17 - protein, 10 aa, Artificial Sequence
  Synthetic Peptide
LLDGTATLRL

SEQ ID NO: 18 - protein, 9 aa, Artificial Sequence
  Synthetic Peptide
YLEPGPVTA

SEQ ID NO: 19 - protein, 10 aa, Artificial Sequence
  Synthetic Peptide
VLYRYGSFSV

SEQ ID NO: 20 - protein, 9 aa, Artificial Sequence
  Synthetic Peptide
RLPRIFCSC

SEQ ID NO: 21 - protein, 9 aa, Artificial Sequence
  Synthetic Peptide
LIYRRRLMK

SEQ ID NO: 22 - protein, 9 aa, Artificial Sequence
  Synthetic Peptide
ALNFPGSQK

SEQ ID NO: 23 - protein, 10 aa, Artificial Sequence
  Synthetic Peptide
SLIYRRRLMK

SEQ ID NO: 24 - protein, 9 aa, Artificial Sequence
  Synthetic Peptide
ALLAVGATK

SEQ ID NO: 25 - protein, 9 aa, Artificial Sequence
  Synthetic Peptide
ALLAVGATK

SEQ ID NO: 26 - protein, 9 aa, Artificial Sequence
  Synthetic Peptide
VYFFLPDHL

SEQ ID NO: 27 - protein, 8 aa, Artificial Sequence
  Synthetic Peptide
SNDGPTLI

SEQ ID NO: 28 - protein, 10 aa, Artificial Sequence
  Synthetic Peptide
VSHSFPHPLY

SEQ ID NO: 29 - protein, 10 aa, Artificial Sequence
  Synthetic Peptide
FLTPKKLQCV

SEQ ID NO: 30 - protein, 10 aa, Artificial Sequence
  Synthetic Peptide
VISNDVCAQV

SEQ ID NO: 31 - protein, 10 aa, Artificial Sequence
  Synthetic Peptide
HSTNGVTRIY

SEQ ID NO: 32 - protein, 9 aa, Artificial Sequence
  Synthetic Peptide
KCDICTDEY

SEQ ID NO: 33 - protein, 11 aa, Artificial Sequence
  Synthetic Peptide
SSDYVIPIGTY

SEQ ID NO: 34 - protein, 9 aa, Artificial Sequence
  Synthetic Peptide
YMDGTMSQV

SEQ ID NO: 35 - protein, 9 aa, Artificial Sequence
  Synthetic Peptide
MLLAVLYCL

SEQ ID NO: 36 - protein, 9 aa, Artificial Sequence
  Synthetic Peptide
AFLPWHRLF

SEQ ID NO: 37 - protein, 9 aa, Artificial Sequence
  Synthetic Peptide
SEIWRDIDF

SEQ ID NO: 38 - protein, 9 aa, Artificial Sequence
  Synthetic Peptide
MSLQRQFLR

SEQ ID NO: 39 - protein, 9 aa, Artificial Sequence
  Synthetic Peptide
SVYDFFVWL

SEQ ID NO: 40 - protein, 9 aa, Artificial Sequence
  Synthetic Peptide
TLDSQVMSL

SEQ ID NO: 41 - protein, 9 aa, Artificial Sequence
  Synthetic Peptide
LLGPGRPYR

SEQ ID NO: 42 - protein, 9 aa, Artificial Sequence
  Synthetic Peptide
ANDPIFVVL

SEQ ID NO: 43 - protein, 10 aa, Artificial Sequence
  HPV T10V peptide
TIHDIILECV

SEQ ID NO: 44 - protein, 10 aa, Artificial Sequence
  HPV Y10T peptide
YMLDLQPETT

SEQ ID NO: 45 - protein, 9 aa, Artificial Sequence
  HPV Y9T peptide
YMLDLQPET

SEQ ID NO: 46 - protein, 9 aa, Artificial Sequence
  HPV L9V peptide
LLMGTLGIV

SEQ ID NO: 47 - protein, 8 aa, Artificial Sequence
  HPV T8I peptide
TLGIVCPI

SEQ ID NO: 48 - protein, 8 aa, Artificial Sequence
  TRP-2 V8L peptide
VYDFFVWL

SEQ ID NO: 49 - protein, 9 aa, Artificial Sequence
  Wild type p53 peptide
KYMCNSSCM

SEQ ID NO: 50 - protein, 9 aa, Artificial Sequence
  Modified p53 peptide
KYICNSSCM

SEQ ID NO: 51 - protein, 22 aa, Artificial Sequence
  Modified p53-PADRE fusion peptide
  misc_feature (3)..(3): Xaa may be cyclohexylalanyl
AKXVAAWTLKAAAKYICNSSCM

SEQ ID NO: 52 - DNA, 429 bp, Homo Sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 53 - protein, 142 aa, Homo Sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 54 - protein, 9 aa, Artificial Sequence
  Survivin HLA-A1 restricted peptide (native)
FEELTLGEF

SEQ ID NO: 55 - protein, 9 aa, Artificial Sequence
  Survivin HLA-A1 restricted pepitde (modified)
FTELTLGEF

SEQ ID NO: 56 - protein, 9 aa, Artificial Sequence
  Survivin HLA-A2 restricted pepitde (native)
LTLGEFLKL

SEQ ID NO: 57 - protein, 10 aa, Artificial Sequence
  Survivin HLA-A3 restricted peptide (native)
RISTFKNWPF

SEQ ID NO: 58 - protein, 10 aa, Artificial Sequence
  Survivin HLA-A3 restricted peptide (modified)
RISTFKNWPK

SEQ ID NO: 59 - protein, 9 aa, Artificial Sequence
  Survivin HLA-A24 restricted peptide (native)
STFKNWPFL

SEQ ID NO: 60 - protein, 9 aa, Artificial Sequence
  Survivin HLA-B7 restricted pepitde (native)
LPPAWQPFL

SEQ ID NO: 61 - protein, 16 aa, Artificial Sequence
  T-helper epitope A16L from tetanus toxin
AQYIKANSKFIGITEL

SEQ ID NO: 62 - protein, 13 aa, Artificial Sequence
  T-helper epitope PADRE
  misc_feature (3)..(3): Xaa may be cyclohexylalanyl
AKXVAAWTLKAAA

SEQ ID NO: 63 - protein, 21 aa, Artificial Sequence
  T-helper epitope F21E from tetanus toxin
FNNFTVSFWLRVPKVSASHLE

SEQ ID NO: 64 - DNA, 20 bp, Artificial Sequence
  CpG oligonucleotide
tccatgacgttcctgacgtt